(19) United States
(12) Patent Application Publication
(10) Pub. No.: US 2024/0190798 A1
(43) Pub. Date: Jun. 13, 2024

(54) PROCESS AND SYSTEMS FOR OBTAINING 1,3-BUTANEDIOL FROM FERMENTATION BROTHS

(71) Applicant: Genomatica, Inc., San Diego, CA (US)

(72) Inventors: Julia Khandurina, San Diego, CA (US); Rachel Diane Pacheco, San Diego, CA (US); Lauri Hannunpoika Suominen, San Diego, CA (US); Rainer Wagester, La Jolla, CA (US)

(21) Appl. No.: 18/469,170

(22) Filed: Sep. 18, 2023

Related U.S. Application Data
(63) Continuation of application No. 16/498,313, filed on Sep. 26, 2019.
(60) Provisional application No. 62/480,270, filed on Mar. 31, 2017.

(51) Int. Cl. C07C 29/80; B01D 3/14; B01D 15/36; C07C 29/94; C07C 31/20

(52) U.S. Cl. CPC C07C 29/80; B01D 3/143; B01D 15/361; C07C 29/94; B01D 2202/20; C07C 31/207

(57) ABSTRACT

Provided herein are bioderived 1,3-butanediol compositions and systems and processes for producing such bioderived 1,3-butanediol compositions.

### CROSS-REFERENCE TO RELATED APPLICATIONS

This application claims the benefit of U.S. Provisional Patent Application No. 62/480,270, filed Mar. 31, 2017 and entitled “Process and Systems for Obtaining 1,3-Butanediol from Fermentation Broths,” the entire contents of which are incorporated by reference herein.

Reference is made to the following provisional and international applications, which are incorporated herein by reference in their entireties: (1) U.S. Provisional Application No. 62/480,208 entitled “3-HYDROXYBUTYRYL-COA DEHYDROGENASE VARIANTS AND METHODS OF USE,” filed Mar. 31, 2017 (Attorney Docket No. 12956-409-888); (2) U.S. Provisional Application No. 62/480,194 entitled “ALDEHYDE DEHYDROGENASE VARIANTS AND METHODS OF USE,” filed Mar. 31, 2017 (Attorney Docket No. 12956-408-888); (3) International Patent Application No. ______ entitled “3-HYDROXYBUTYRYL-COA DEHYDROGENASE VARIANTS AND METHODS OF USE,” filed on even date herewith (Attorney Docket No. 12956-409-228); and (4) International Patent Application No. ______ entitled “ALDEHYDE DEHYDROGENASE VARIANTS AND METHODS OF USE,” filed on even date herewith (Attorney Docket No. 12956-408-228).

### BACKGROUND

The present disclosure relates generally to compositions produced by biosynthetic processes, as well as the processes and systems for producing such compositions.

1,3-BG (which also can be referred to as BG, 1,3-butanediol, 1,3-BDO, 13-BDO, 1,3-butylene glycol, or butylene glycol) is a four carbon diol traditionally produced in a chemical process from petroleum derived acetylene via its hydration (“petro-BG”). The resulting acetaldehyde is then converted to 3-hydroxybutyraldehyde which is subsequently reduced to form 1,3-BG. 1,3-BG is used in many industrial processes, e.g., as an organic solvent for food flavoring agents and as a reagent for the production of polyurethane and polyester resins. Due to its generally low-toxic, low-irritant properties, 1,3-BG also finds increasing use in the cosmetics industry. Here, 1,3-BG is especially useful as an odorless cosmetic grade ingredient.

While cosmetic grade petro-BG and processes for producing and storing cosmetic grade petro-BG are available to the cosmetics industry, there remains a need for bioderived 1,3-BG (“bio-BG”) for cosmetic and food applications as well as processes and systems for producing such bio-BG.

### SUMMARY

In one aspect, provided herein is bioderived 1,3-butylene glycol (1,3-BG), whereby the bioderived 1,3-BG includes detectable levels of one or more compounds selected from 3-hydroxy-butanal, 4-hydroxy-2-butanone, 4-(3-hydroxybutoxy)butan-2-one, 4-((4-hydroxybutan-2-yl)oxy)-butan-2-one, 1,2-propanediol, 1,3-propanediol or 2,3-butanediol.

In some embodiments, the bioderived 1,3-BG includes detectable levels of 3-hydroxy-butanal, 4-hydroxy-2-butanone, 4-(3-hydroxybutoxy)butan-2-one or 4-((4-hydroxybutan-2-yl)oxy)-butan-2-one.

In some embodiments, the bioderived 1,3-BG includes higher levels than petro-BG of one or more compound selected from 3-hydroxy-butanal, 4-hydroxy-2-butanone, 4-(3-hydroxybutoxy)butan-2-one or 4-((4-hydroxybutan-2-yl)oxy)-butan-2-one.

In some embodiments, the chiral purity of the bioderived 1,3-BG is 95% or more, 96% or more, 97% or more, 98% or more, 99.0% or more, 99.1% or more, 99.2% or more, 99.3% or more, 99.4% or more, 99.5% or more, 99.6% or more, 99.7% or more, 99.8% or more, or 99.9% or more.

In some embodiments, the bioderived 1,3-BG has a chemical purity of 99.0% or more, 99.1% or more, 99.2% or more, 99.3% or more, 99.4% or more, 99.5% or more, 99.6% or more, 99.7% or more, 99.8% or more, or 99.9% or more.

In some embodiments, the bioderived 1,3-BG has more R-enantiomer than S-enantiomer.

In some embodiments, the bioderived 1,3-BG has a chiral purity of 95% or more and a chemical purity of 99.0% or more.

In some embodiments, the bioderived 1,3-BG has a chiral purity of 99.0% or more and a chemical purity of 99.0% or more.

In some embodiments, the bioderived 1,3-BG has a chiral purity of 99.5% or more and a chemical purity of 99.0% or more.

In some embodiments, the bioderived 1,3-BG is industrial grade or cosmetic grade.

In some embodiments, the bioderived 1,3-BG includes levels of 5 ppm or more, 10 ppm or more, 20 ppm or more, 30 ppm or more, 40 ppm or more or more, 50 ppm or more, 100 ppm or more, 200 ppm or more, 300 ppm or more, 400 ppm or more, 500 ppm or more, 600 ppm or more, 700 ppm or more, 800 ppm or more, 900 ppm or more, 1,000 ppm or more, 1,500 ppm or more, or 2,000 ppm or more of the compound.

In some embodiments, the bioderived 1,3-BG includes detectable levels of a compound characterized by a mass spectrum according to FIG. 3 or FIG. 4.

In some embodiments, the bioderived 1,3-BG includes a compound detectable in a GC-MS chromatogram as a peak eluting with a relative retention time of between 0.97-0.99, whereby the relative retention time of 1,3-BG is 1.0.

In some embodiments, the bioderived 1,3-BG includes a compound detectable in a GC-MS chromatogram as a peak eluting with a relative retention time of between 0.94-0.96, whereby the relative retention time of 1,3-BG is 1.0.

In some embodiments, the bioderived 1,3-BG does not include detectable levels of one or more contaminants of petro-BG detectable in an GC-MS chromatogram as peaks eluting with a relative retention time of between 0.8-0.95, whereby the relative retention time of 1,3-BG is 1.0.

In some embodiments, the bioderived 1,3-BG includes at least 2-fold, at least 3-fold, at least 4-fold, at least 5-fold, at least 6-fold, at least 7-fold, at least 8-fold, at least 9-fold, or at least 10-fold lower levels of one or more contaminants of petro-BG detectable in an GC-MS chromatogram as peaks eluting with a relative retention time of between 0.8-0.95, whereby the relative retention time of 1,3-BG is 1.0.

In some embodiments, chemical purity of the bioderived 1,3-BG is 99% or higher, the overall level of heavies is 0.8% or less, and the overall level of lights is 0.2% or less.

In some embodiments, the UV absorbance between 220 nm and 260 nm of the bioderived 1,3-BG is at least at least 2-fold, at least 3-fold, at least 4-fold, at least 5-fold, at least 6-fold, at least 7-fold, at least 8-fold, at least 9-fold, or at least 10-fold lower than the UV absorbance of petro-BG.

In some embodiments, the bioderived 1,3-BG does not comprise detectable levels of 1-4-(4-methyl-1,3-dioxan-2-yl)propan-2-one.

In some embodiments, the bioderived 1,3-BG includes at least at least 2-fold, at least 3-fold, at least 4-fold, at least 5-fold, at least 6-fold, at least 7-fold, at least 8-fold, at least 9-fold, or at least 10-fold lower levels of 1-4-(4-methyl-1,3-dioxan-2-yl)propan-2-one than petro-BG.

In some embodiments, the detectable levels are analyzed by gas-chromatograph coupled mass spectrometry or liquid chromatography coupled mass spectrometry.

In some embodiments, the bioderived 1,3-BG has a chiral purity of 55% or more.

In another aspect, provided herein is a process of purifying bioderived 1,3-BG including: (a) subjecting a first bioderived 1,3-BG-containing product stream to a first column distillation procedure to remove materials with a boiling point higher than bioderived 1,3-BG, as a first high boilers stream, to produce a second bioderived 1,3-BG-containing product stream; (b) subjecting the second bioderived 1,3-BG-containing product stream to a second column distillation procedure to remove materials with a boiling point lower than bioderived 1,3-BG, to produce a third bioderived 1,3-BG-containing product stream; and (c) subjecting the third bioderived 1,3-BG-containing product stream to a third column distillation procedure to remove materials with boiling points higher than bioderived 1,3-BG as a second high-boilers stream, to produce a purified bioderived 1,3-BG product.

In some embodiments, the process further includes subjecting a crude bioderived 1,3-BG mixture to a dewatering column distillation procedure to remove materials with a boiling point lower than bioderived 1,3-BG from the crude bioderived 1,3-BG mixture to produce the first bioderived 1,3-BG-containing product stream of (a).

In some embodiments, the process further includes subjecting crude bioderived 1,3-BG to polishing ion exchange to produce the first bioderived 1,3-BG-containing product stream of (a).

In some embodiments, the purified bioderived 1,3-BG product includes detectable levels of one or more compounds selected from the group consisting of 3-hydroxy-butanal, 4-hydroxy-2-butanone, 4-(3-hydroxybutoxy)butan-2-one, 4-((4-hydroxybutan-2-yl)oxy)-butan-2-one, 1,2-propanediol, 1,3-propanediol and 2,3-butanediol.

In some embodiments, the purified bioderived 1,3-BG product does not include a detectable level, or only includes a low level, of 1-4-(4-methyl-1,3-dioxan-2-yl)propan-2-one.

In some embodiments, the process further includes adding a base to a bioderived 1,3-BG-containing product stream before or after any one of (a), (b), or (c).

In some embodiments, the base is added to the bioderived 1,3-BG-containing product stream after (a).

In some embodiments, the process further includes treating a bioderived 1,3-BG containing product stream with a hydrogenation reaction before or after any one of (a), (b), or (c)

In some embodiments, the the second bioderived 1,3-BG containing product stream is treated with a hydrogenation reaction prior to performing (b).

In some embodiments, the hydrogenation reaction reduces the concentration of 3-hydroxy-butanal or 4-hydroxy-2-butanone in the second bioderived 1,3-BG containing product stream by 50% or more, 60% or more, 70% or more, 80% or more, 90% or more, or 95% or more.

In some embodiments, the hydrogenation reaction reduces the UV absorption at 270 nm or at 220 nm in the second bioderived 1,3-BG containing product stream by 50% or more, 60% or more, 70% or more, 80% or more, 90% or more, or 95% or more.

In some embodiments, the purified bioderived 1,3-BG product is collected as a distillate of the third column distillation procedure.

In some embodiments, the process further includes contacting the distillate of the third column distillation procedure with activated carbon to produce the purified bioderived 1,3-BG product.

In some embodiments, the process further includes contacting the second bioderived 1,3-BG containing product stream with activated carbon prior to performing step (c).

In some embodiments, the contacting with activated carbon reduces the concentration of 3-hydroxy-butanal or 4-hydroxy-2-butanone in the second bioderived 1,3-BG containing product stream by 50% or more, 60% or more, 70% or more, 80% or more, 90% or more, or 95% or more.

In some embodiments, the process further includes contacting the second bioderived 1,3-BG containing product stream with sodium borohydride (NaBH₄) prior to performing step (c).

In some embodiments, the contacting with NaBH₄ reduces the UV absorption at 270 nm or at 220 nm in the second bioderived 1,3-BG containing product stream by 50% or more, 60% or more, 70% or more, 80% or more, 90% or more, or 95% or more.

In some embodiments, bioderived 1,3-BG has a chiral purity of 55% or more.

In some embodiments, the purified bioderived 1,3-BG product has a chemical purity of 99.0% or more.

In another aspect, provided herein is a system for purifying bioderived 1,3-BG, including a first distillation column receiving a first bioderived 1,3-BG containing product stream generating a first stream of materials with boiling points higher than 1,3-BG, and a second bioderived 1,3-BG-containing product stream; a second distillation column receiving the second bioderived 1,3-BG-containing product stream generating a stream of materials with boiling points lower than 1,3-BG, and a third bioderived 1,3-BG-containing product stream; and a third distillation column receiving the third 1,3-BG-containing product stream at a feed point and generating a second stream of materials with boiling points higher than 1,3-BG, and a fourth bioderived 1,3-BG-containing product stream comprising a purified bioderived 1,3-BG product.

In some embodiments, the fourth bioderived 1,3-BG-containing product stream consists essentially of a bioderived 1,3-BG provided herein.

In some embodiments, the system includes a polishing column receiving a crude bioderived 1,3-BG mixture generating a crude bioderived 1,3-BG mixture of reduced salt content.

In some embodiments, the polishing column is an ion exchange chromatography column.

In some embodiments, the system includes a dewatering column receiving a crude bioderived 1,3-BG mixture generating a stream of materials with boiling points lower than 1,3-BG and the first bioderived 1,3-BG-containing product stream.

In some embodiments, the bioderived 1,3-BG is produced by a process provided herein or by a system provided herein.

### BRIEF DESCRIPTION OF THE DRAWINGS

FIG. 1 shows a chromatogram illustrating results of an exemplary gas-chromatography mass spectrometry (GC-MS) analysis of bio-BG (downward pointing trace) and of industrial-grade and cosmetic-grade petro-BG (upward pointing traces) at 2-fold sample dilutions.

FIG. 2 shows a chromatogram illustrating results of an exemplary GC-MS analysis of bio-BG (downward pointing trace) and of industrial-grade and cosmetic-grade petro-BG (upward pointing traces) at 20-fold sample dilutions.

FIG. 3 shows a representative mass-spectrum of bio-BG heavies compound #7, with proposed interpretations of certain mass fragments indicated.

FIG. 4 shows a representative mass-spectrum of bio-BG heavies compound #9, with proposed interpretations of certain mass fragments indicated.

FIG. 5 shows proposed chemical structures of bio-BG heavies compounds #7 and #9 and illustrates proposed mass spectrometry fragmentation patterns of bio-BG heavies compounds #7 and #9.

FIG. 6A shows an exemplary extracted ion chromatogram for m/z 115 of a bio-BG sample.

FIG. 6B shows an exemplary extracted ion chromatogram for m/z 115 of a petro-BG sample.

FIG. 7 shows exemplary liquid-chromatography mass spectrometry (LC-MS) chromatograms (TIC: total ion current) of a bio-BG sample (top panel), a cosmetic-grade petro-BG sample (middle panel), and an industrial-grade petro-BG sample (bottom panel).

FIG. 8A shows exemplary LC-MS chromatograms of bio-BG (total ion current (TIC): top panel; extracted ion current (XIC) chromatogram: second panel from top), of cosmetic-grade petro-BG XIC (third panel from top), and of industrial-grade petro-BG XIC (bottom panel).

FIG. 8B shows an exemplary mass-spectrum of C₈H₁₆O₃ (MW 160) components of bio-BG and petro-BG (cosmetic and industrial grade) observed at LC retention times of 6.0-6.7 minutes, with proposed interpretations of certain mass fragments indicated.

FIG. 9A shows exemplary LC-MS chromatograms of bio-BG (total ion current (TIC): top panel; extracted ion current (XIC) chromatogram: second panel from top), of cosmetic-grade petro-BG XIC (third panel from top), and of industrial-grade petro-BG XIC (bottom panel).

FIG. 9B shows an exemplary mass-spectrum of C₈H₁₄O₃ (MW 158) components of petro-BG (cosmetic and industrial grade) observed at LC retention times of 7.3 minutes, with proposed interpretations of certain mass fragments indicated.

FIG. 10 shows a chromatogram illustrating results of an exemplary gas-chromatography mass spectrometry and olfactory (GC-MS/O) analysis of cosmetic grade petro-BG. The upper trace and upward pointing peaks represent results of an olfactory analysis of GC-MS fractions performed by a trained individual. The lower trace and downward pointing peaks represent the mass spectrum of the GC-MS (total ion current (TIC)).

FIG. 11 shows a chromatogram illustrating results of an exemplary gas-chromatography mass spectrometry and olfactory (GC-MS/O) analysis of bioderived 1,3-BG, produced using a process or system provided herein. The upper trace and upward pointing peaks represent results of an olfactory analysis of GC-MS fractions performed by a trained individual. The lower trace and downward pointing peaks represent the mass spectrum of the GC-MS (total ion current (TIC)).

FIG. 12 shows chemical structures illustrating the chemical reaction of 3-hydroxy butanal (3-OH-butanal) to crotonaldehyde (Cr-Ald) and of 4-hydroxy-butanone (4-OH-2-butanone) to methyl-vinyl-ketone (MVK) observed or believed to be observed during distillation of 1,3-BG.

FIG. 13 shows a graph of UV-VIS absorption spectra of petro-BG and bio-BG preparations. #1: bio-BG sample treated with activated carbon after a final distillation; #2: bio-BG feed to a final distillation prior to base addition; #3 and #4 samples of commercially available cosmetic grade petro-BG; #5 and #6 samples of commercially available industrial grade petro-BG; #7 sample of bio-BG treated with base addition in reboiler (“cut 4”); #8: bio-BG preparation #7 further treated with NaBH₄.

FIG. 14A, FIG. 14B, FIG. 14C and FIG. 14D show graphs illustrating results of a hydrogenation experiment with bio-BG. In FIG. 14A, UV absorption of a bio-BG sample is plotted against the hydrogenation time for four nickel hydrogenation catalysts (Raney, NiSAT320®, NiSAT330®, and NiSAT340®). In FIG. 14B, the concentration of 4-hydroxy-butanone found in a bio-BG sample is plotted against the hydrogenation time for four nickel hydrogenation catalysts. In FIG. 14C, the concentration of isopropylalcohol (IPA) found in a bio-BG sample is plotted against the hydrogenation time for four nickel hydrogenation catalysts. In FIG. 14D, the concentration of n-butanol found in a bio-BG sample is plotted against the hydrogenation time for four nickel hydrogenation catalysts.

FIG. 15A, FIG. 15B, and FIG. 15C show graphs illustrating exemplary distillation systems provided herein.

FIG. 16 shows a graph illustrating an exemplary ASPEN model of a 4-column distillation train such as provided herein.

### DETAILED DESCRIPTION

Commercially, 1,3-BG is typically produced by chemically converting acetaldehyde (derived from petroleum or from ethanol) to 3-hydroxybutyraldehyde, which is subsequently reduced to form petroleum-derived 1,3-BG (“petro-BG”). This chemically produced petro-BG usually forms a racemic mixture of equimolar ratios of 1,3-BG R- and S-enantiomers. Using the 1,3-BG racemate, methods have been disclosed to isolate each chiral form from petro-BG. Such isolations methods, however, have generally proven to be very inefficient (e.g. enzyme conversion of racemate) or very expensive and difficult to scale-up to industrial-scale production (e.g. chiral chromatography).

The Applicant has recognized that there remains a need for a bioderived 1,3-BG (“bio-BG”) that is highly pure for use in the cosmetic and food industries. Specifically, Applicant identified a need for the R-enantiomer of 1,3-BG for food, nutraceutical, pharmaceutical, and other applications where the R-enantiomer is believed to be more physiologically effective than the S-enantiomer, e.g., for applications in humans and animals generally (e.g., farm animals or domestic animals). In particular, Applicant identified a need for an R-enantiomer of 1,3-BG having an improved purity profile, e.g., relative to typical commercially available petro-BG racemate preparations. Processes allowing for the economically effective production of the R-enantiomer of 1,3-BG are wanted to produce 1,3-BG at a commercial-scale for applications in the cosmetic and other industries, e.g., in the food or pharmaceutical industry.

The instant disclosure is further based, in part, on the realization that petro-BG and bio-BG have different odor characteristics and that the different odor of petro-BG and bio-BG are due to different impurities commonly present in petro-BG and bio-BG preparations.

The instant disclosure is further based, in part, on the realization that highly chemically pure (e.g., overall purity) bioderived 1,3-BG and enriched or highly chirally pure R-enantiomer of bioderived 1,3-BG can have different or preferred odor characteristics, or improved physiological properties (e.g., observable in an in vitro assay or in vivo) relative to racemic 1,3-BG mixtures or petro-BG generally (e.g., cosmetic grade or industrial grade).

Provided herein are purified bio-BG products as well as processes and systems for producing such purified bio-BG products.

In one aspect, bioderived 1,3-butylene glycol (1,3-BG) is provided (“bio-BG”). In some embodiments, the bioderived 1,3-BG has a different odor than chemically derived 1,3-BG, such as 1,3-BG derived from processing petroleum or acetaldehyde. In some embodiments, the bioderived 1,3-BG does not have a characteristic off-odor that is typically found in industrial grade bio-BG. In some embodiments, the bioderived 1,3-BG has an improved odor compared to petro-BG, e.g., as determined in a sensory test by a trained odor panel. In some embodiments, the improved odor of bio-BG is characterized as “sweet,” e.g., by a trained odor panel. In some embodiments, the bioderived 1,3-BG is cosmetic grade. In some embodiments, the cosmetic grade bioderived 1,3-BG has an improved odor characteristic (e.g., “sweet” odor) compared to petro-BG. In another aspect, provided herein are systems for purifying bioderived 1,3-BG. In another aspect, provided herein are processes for purifying bioderived 1,3-BG.

In some embodiments, the bioderived 1,3-BG is a racemate, or mixture of R- and S-enantiomers of 1,3-BG (e.g., CAS No. 107-88-0).

In some embodiments, the 1,3-BG racemate is an equimolar mixture of R- and S-enantiomers of 1,3-BG.

In some embodiments, the 1,3-BG racemate has more R-enantiomer than S-enantiomer of 1,3-BG. In some embodiments, the 1,3-BG racemate has essentially only R-enantiomer (e.g., >95%, >96%, >97%, >98%, >99%, >99.1%, >99.2%, >99.3%, >99.4%, >99.5%, >99.6%, >99.7%, >99.8%, or >99.9% of R-enantiomer). In some embodiments, the bioderived 1,3-BG has essentially only R-enantiomer (e.g., 100% enantiomer; CAS No. 6290-03-5) and no S-enantiomer is detectable, e.g., by GC-MS or LC-MS. In some embodiments, the 1,3-BG racemate is enriched in R-enantiomer, that is, includes more R-enantiomer than S-enantiomer. For example, the 1,3-BG racemate can include 55% or more R-enantiomer and 45% or less S-enantiomer. For example, the 1,3-BG racemate can include 60% or more R-enantiomer and 40% or less S-enantiomer. For example, the 1,3-BG racemate can include 65% or more R-enantiomer and 35% or less S-enantiomer. For example, the 1,3-BG racemate can include 70% or more R-enantiomer and 30% or less S-enantiomer. For example, the 1,3-BG racemate can include 75% or more R-enantiomer and 25% or less S-enantiomer. For example, the 1,3-BG racemate can include 80% or more R-enantiomer and 20% or less S-enantiomer. For example, the 1,3-BG racemate can include 85% or more R-enantiomer and 15% or less S-enantiomer. For example, the 1,3-BG racemate can include 90% or more R-enantiomer and 10% or less S-enantiomer. For example, the 1,3-BG racemate can include 95% or more R-enantiomer and 5% or less S-enantiomer.

In some preferred embodiments, the bioderived 1,3-BG is enriched for the R-enantiomer. Therefore, even if not expressly stated, in each instance in this disclosure referring to bioderived 1,3-BG provided herein, or alternative terms, such as bio 1,3-butylene glycol, bio 1,3-BG, bio-BG, bio 13-BDO, bio 1,3-BDO, bio-butylene glycol, or bio 1,3-butanediol, an expressly preferred embodiment is the R-enantiomer. An especially preferred composition is highly chirally pure, ≥99% R-enantiomer, and highly chemically pure, e.g., ≥99%, optionally with specific impurities present at or below a preferred level, as described in more detail elsewhere herein. Additional compositions provided herein are enriched in the R-enantiomer, e.g., include ≥55% R-enantiomer, ≥60% R-enantiomer, ≥65% R-enantiomer, ≥70% R-enantiomer, ≥75% R-enantiomer, ≥80% R-enantiomer, ≥85% R-enantiomer, ≥90% R-enantiomer, or ≥95% R-enantiomer, and can be highly chemically pure, e.g., ≥99%, optionally with specific impurities present at or below a preferred level, as described in more detail elsewhere herein.

The bioderived 1,3-BG provided herein, especially R-enantiomer compositions, and, preferably, highly chemically pure and chirally pure (e.g., ≥95% chemically pure and ≥99% chirally pure, or, more preferably, ≥99% or >99.5% chemically pure and >99.5% chirally pure), as well as compositions that are enriched in the R-enantiomer and are highly chemically pure and chirally pure (e.g., ≥95% chemically pure and ≥50% chirally pure, or ≥95% chemically pure and ≥55% chirally pure) can find use in food, nutraceutical, pharmaceutical, cosmetic and industrial applications. For example, bioderived 1,3-BG, can be reacted with an acid, either in vivo or in vitro, e.g., enzymatically using a lipase, to convert the bioderived 1,3-BG to an ester. Such esters can have nutraceutical, medical and food uses. Specifically, such bioderived 1,3-BG esters can be advantaged when the R-enantiomer of bioderived 1,3-BG, or bioderived 1,3-BG that is enriched in the R-enantiomer, is used for ester formation (e.g., compared to use of the S-enantiomer or a racemic mixture of petro-BG, e.g., made from petroleum or from ethanol, e.g., through the acetaldehyde chemical synthesis route) since chiral ester forms including the 1,3-BG R-enantiomer are preferred energy sources of humans and animals. Examples include the ketone ester (R)-3-hydroxybutyl-R-1,3-butanediol monoester, which has been recognized by the United States Food and Drug Administration (FDA) as being generally safe (GRAS approval) and (R)-3-hydroxybutyrate glycerol monoester or diester. The ketone esters can be delivered orally and, in vivo, release R-1,3-butylene glycol that can be used, e.g., by the human body. See, e.g., WO2013150153 (“Ketone Bodies and Ketone Body Esters for Maintaining or Improving Muscle Power Output.”), the entire contents of which are incorporated by reference herein. Thus the instant disclosure of highly chirally pure and highly chemically pure R-enantiomer compositions of 1,3-BG are particularly useful for applications in the food and pharmaceutical industry. Bioderived 1,3-BG (e.g., the R-enantiomer of bioderived 1,3-BG, or bioderived 1,3-BG that is enriched in the R-enantiomer) has further food related uses, including use as a food ingredient, a flavoring agent, a solvent or solubilizer for flavoring agents, a stabilizer, an emulsifier, and an anti-microbial agent and preservative. Bioderived 1,3-BG (e.g., the R-enantiomer of bioderived 1,3-BG, or bioderived 1,3-BG that is enriched in the R-enantiomer) can also be used in the pharmaceutical industry as a parenteral drug solvent. Additionally, bioderived 1,3-BG (e.g., the R-enantiomer of bioderived 1,3-BG, or bioderived 1,3-BG that is enriched in the R-enantiomer) finds use in cosmetics as an ingredient, such as an emollient, a humectant, an additive that can prevent crystallization of insoluble ingredients, a solubilizer for less-water-soluble ingredients, such as fragrances, and as an anti-microbial agent and preservative. For example, bioderived 1,3-BG (e.g., the R-enantiomer of bioderived 1,3-BG, or bioderived 1,3-BG that is enriched in the R-enantiomer) can be used as a humectant, especially in hair sprays and setting lotions. Bioderived 1,3-BG (e.g., the R-enantiomer of bioderived 1,3-BG, or bioderived 1,3-BG that is enriched in the R-enantiomer) can reduce loss of aromas from essential oils, preserve against spoilage by microorganisms, and be used as a solvent for benzoates. Bioderived 1,3-BG can, e.g., be used at concentrations from 0.1% or less to 50% or more. Bioderived 1,3-BG (e.g., the R-enantiomer of bioderived 1,3-BG, or bioderived 1,3-BG that is enriched in the R-enantiomer) can be used in hair and bath products, eye and facial makeup, fragrances, personal cleanliness products, and shaving and skin care preparations. See, e.g., Cosmetic Ingredient Review Board Report: “Final Report on the Safety Assessment of Butylene Glycol, Hexylene Glycol, Ethoxy diglycol, and Dipropylene Glycol,” Journal of the American College of Toxicology, Volume 4, Number 5, 1985 (“Report”). The Report, which is hereby incorporated by reference herein in its entirety, provides specific uses and concentrations of butylene glycol in cosmetics. See, e.g., Report, Table 2 (“Product Formulation Data”). While the Report describes uses of petro-BG racemates, bioderived 1,3-BG, and especially R-enantiomer enriched preparations provided herein, are expected to be superior products to petro-BG racemates, at least because of their improved purity profile and preferable odor characteristics.

As used herein, the term “crude bioderived 1,3-BG mixture” means a mixture of bioderived 1,3-BG (1,3-BDO) that is or includes about 50% to 90% bioderived 1,3-BG and 50% to 1% water with one or more other impurities that are derived from a fermentation process. In some embodiments, the crude bioderived 1,3-BG mixture is about 75% to 85% 1,3-BG or more with 1% to 25% water with one or more other impurities derived from a fermentation process. In some embodiments, the crude bioderived 1,3-BG mixture is about 80% to 85% 1,3-BG with 1% to 20% water with one or more other impurities derived from a fermentation process. The crude bioderived 1,3-BG mixture can be or include partially purified bioderived 1,3-BG, e.g., a mixture including bioderived 1,3-BG that has been partially purified using one or more processes.

As used herein, the term “bioderived 1,3-BG-containing product stream” means material that leaves a procedure and contains the majority of bioderived 1,3-BG that entered the procedure.

As used herein, the term “bioderived 1,3-BG product” means a mixture that contains bioderived 1,3-BG, and has been subjected to at least one procedure to increase the content of bioderived 1,3-BG or decrease the content of an impurity. The term bioderived 1,3-BG product can include a crude bioderived 1,3-BG mixture or partially purified bioderived 1,3-BG, however, the bioderived 1,3-BG and water content of a bioderived 1,3-BG product can be higher or lower than a crude bioderived 1,3-BG mixture or partially purified bioderived 1,3-BG.

As used herein, the term “bioderived 1,3-BG in a fermentation broth” means a fermentation broth that contains bioderived 1,3-BG produced by culturing a non-naturally occurring microbial organism capable of producing bioderived 1,3-BG in a suitable culturing medium. The terms “bioderived 1,3-BG” and “bio-BG” are used interchangeably herein.

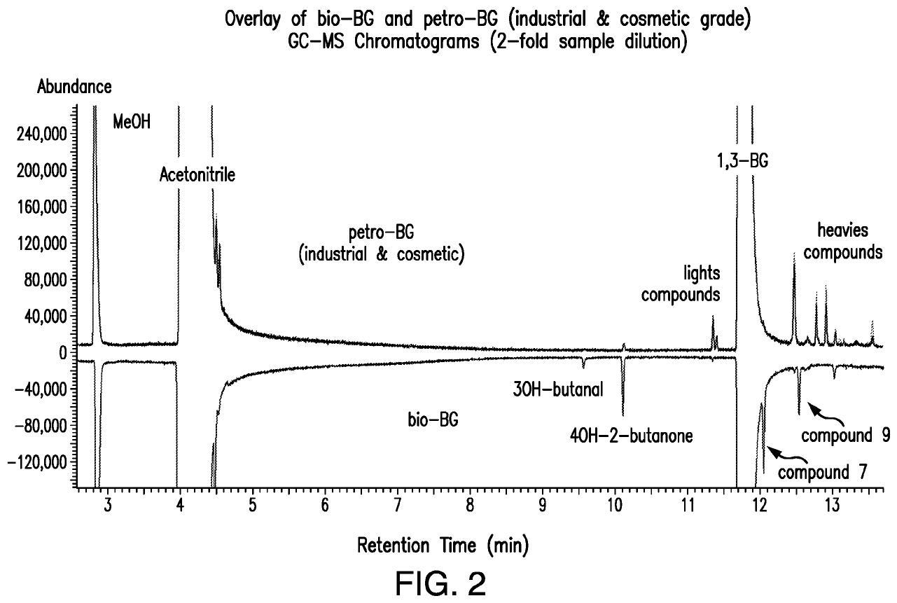

As used herein, the term “bioderived” means produced from or synthesized by a biological organism and can be considered a renewable resource since it can be generated by a biological organism. Such a biological organism, in particular the microbial organisms for use in the compositions, systems, and methods provided and disclosed herein, can utilize feedstock or biomass, such as, sugars or carbohydrates, preferably dextrose or glucose, obtained from an agricultural, plant, bacterial, or animal source; or other renewable sources such as synthesis gas (CO, CO₂ and/or H₂). Coal products can also be used as a carbon source for a biological organism to synthesize a bio-based product such as provided herein. Alternatively, the biological organism can utilize atmospheric carbon. As used herein, the term “biobased” means a product as described above that is composed, in whole or in part, of a bioderived compound provided herein. A biobased or bioderived product is in contrast to a petroleum derived product, wherein such a product is derived from or chemically synthesized from petroleum or a petrochemical feedstock. A preferred microbial route to bioderived 1,3-BG is described, e.g., in WO2010127319A2, the entire contents of which are incorporated by reference herein. Specifically, WO2010127319A2 described biosynthetic pathways including a 3-hydroxybutyryl-CoA dehydrogenase, such as a pathway from acetoacetyl-CoA to 1,3-butanediol (see, e.g., FIG. 2, step H). In one embodiment the 3-hydroxybutyryl-CoA dehydrogenase is modified to have specificity for an R enantiomer. Reference is also made to the following provisional applications, which are incorporated herein by reference in their entireties: (1) U.S. Provisional Application No. 62/480,208 entitled “3-HYDROXYBUTYRYL-COA DEHYDROGENASE VARIANTS AND METHODS OF USE,” filed Mar. 31, 2017 (Attorney Docket No. 12956-409-888); (2) U.S. Provisional Application No. 62/480,194 entitled, “ALDEHYDE DEHYDROGENASE VARIANTS AND METHODS OF USE,” filed Mar. 31, 2017 (Attorney Docket No. 12956-408-888); (3) International Patent Application No. ______ entitled “3-HYDROXYBUTYRYL-COA DEHYDROGENASE VARIANTS AND METHODS OF USE,” filed on even date herewith (Attorney Docket No. 12956-409-228); and (4) International Patent Application No. ______ entitled, “ALDEHYDE DEHYDROGENASE VARIANTS AND METHODS OF USE,” filed on even date herewith (Attorney Docket No. 12956-408-228).

As used herein, the term “detectable levels” means the level of an analyte (e.g., 1,3-BG or an impurity in a 1,3-BG product) that can be detected with an analytical method over a background observed with the analytical method in the absence of the analyte. The analytical method can include detection by an analytical device or instrument, e.g., GC-MS, LC-MS, or a sensory detection by an individual, e.g., an olfactory detection or characterization of an analyte by a trained individual or by a panel of trained individuals. A detectable level can be qualitative (e.g., an analyte is determined to be “present” or “absent” in a sample) or quantitative (e.g., an analyte is determined to be present at 100 ppm, e.g., by weight, in a sample). In some embodiments, an analyte is at a detectable level if it produces a signal intensity of 2σ- or more or 3σ- or more above a background noise observed in the absence of the analyte, e.g., the background noise observed in a GC-MS assay or an LC-MS assay (e.g., total ion current (TIC) or extracted ion current (XIC)).

As used herein, the term “low levels” means the analyte is present at a level close to the limit of detection of an analytical method, e.g., less than 5σ-, less than 4σ-, or less than 3σ-above a background noise observed with the analytical method in the absence of the analyte.

As used herein, the term “lights” refers to compounds in a 1,3-BG sample (e.g., a bio-BG or petro-BG sample) that elute at earlier retention times than 1,3-BG, e.g., in a GC-MS chromatogram or an LC-MS chromatogram.

As used herein, the term “heavies” refers to compounds in a 1,3-BG sample (e.g., a bio-BG or petro-BG sample) that elute at later retention times than 1,3-BG, e.g., in a GC-MS chromatogram or an LC-MS chromatogram.

As used herein, the term “purity” refers to either chemical or chiral purity, or both.

As used herein, the term “chiral purity” is meant, e.g., the fraction of an enantiomer (e.g., R-enantiomer or S-enantiomer) in a racemic mixture, e.g., of 1,3-BG. For example, in a 99% chirally pure bioderived 1,3-BG, 99% of 1,3-BG molecules may be the R-enantiomer and 1% of 1,3-BG molecules may be the S-enantiomer, or vice versa. A 99% chirally pure bioderived 1,3-BG can have any chemical purity. For example, a 99% chirally pure bioderived 1,3-BG can have a chemical purity of 95% (e.g., by weight). A 99% chirally pure bioderived 1,3-BG that is 95% chemically pure can, e.g., include 95% 1,3-BG, e.g., by weight, including R-enantiomer and or S-enantiomer 1,3-BG, and 5% other contaminants, such as “heavies” or “lights,” which also respectively can be referred to as “bio-BG heavies” and “bio-BG lights.”

As used herein the term “chemical purity” means the fraction of, e.g., 1,3-BG in a 1,3-BG composition (e.g., by weight). For example, a 95% chemically pure 1,3-BG can have 95% of 1,3-BG (e.g., by weight) and 5% other contaminants, such as “heavies” or “lights.” A 95% chemically pure 1,3-BG can have any chiral purity. For example, a 95% chemically pure 1,3-BG can be 99% chirally pure, e.g., have 99% of 1,3-BG in the R-enantiomer form and 1% of 1,3-BG in the S-enantiomer form.

In some embodiments, the bioderived 1,3-BG has a purity level (e.g., chemical or chiral purity, or both chemical and chiral purity) of at least 55%, at least 60%, at least 65%, at least 70%, at least 75%, at least 80%, at least 85%, at least 90%, at least 95%, or at least 99%, e.g., on a weight/weight basis. In some embodiments, the bioderived 1,3-BG has a purity level (e.g., chemical or chiral purity, or both chemical and chiral purity) of at least 95%, at least 96%, at least 97%, at least 98%, or at least 99%. In some embodiments, the bioderived 1,3-BG has a purity level (e.g., chemical or chiral purity, or both chemical and chiral purity) of at least 99.0%, at least 99.1%, at least 99.2%, at least 99.3%, at least 99.4%, at least 99.5%, at least 99.6%, at least 99.7%, at least 99.8%, or at least 99.9%.

In some embodiments, the bioderived 1,3-BG has a chemical purity of 99.0% (e.g., 99.1, 99.2, 99.3, 99.4, 99.5, 99.6, 99.7, 99.8, 99.9, percent or more). In some embodiments, the bioderived 1,3-BG has less than 0.5% of water. In some embodiments, the 99.0% or more chemically pure 1,3-BG has a chiral purity of 55.0% or more (e.g., R-enantiomer). In some embodiments, the 99.0% or more chemically pure 1,3-BG has a chiral purity of 60.0% or more (e.g., R-enantiomer). In some embodiments, the 99.0% or more chemically pure 1,3-BG has a chiral purity of 65.0% or more (e.g., R-enantiomer). In some embodiments, the 99.0% or more chemically pure 1,3-BG has a chiral purity of 70.0% or more (e.g., R-enantiomer). In some embodiments, the 99.0% or more chemically pure 1,3-BG has a chiral purity of 75.0% or more (e.g., R-enantiomer). In some embodiments, the 99.0% or more chemically pure 1,3-BG has a chiral purity of 80.0% or more (e.g., R-enantiomer). In some embodiments, the 99.0% or more chemically pure 1,3-BG has a chiral purity of 85.0% or more (e.g., R-enantiomer). In some embodiments, the 99.0% or more chemically pure 1,3-BG has a chiral purity of 90.0% or more (e.g., R-enantiomer).

In some embodiments, the 99.0% or more chemically pure 1,3-BG has a chiral purity of 95.0% or more (e.g., R-enantiomer). In some embodiments, the 99.0% or more chemically pure 1,3-BG has a chiral purity of 96.0% or more (e.g., R-enantiomer). In some embodiments, the 99.0% or more chemically pure 1,3-BG has a chiral purity of 97.0% or more (e.g., R-enantiomer). In some embodiments, the 99.0% or more chemically pure 1,3-BG has a chiral purity of 98.0% or more (e.g., R-enantiomer). In some embodiments, the 99.0% or more chemically pure 1,3-BG has a chiral purity of 99.0% or more (e.g., R-enantiomer). In some embodiments, the 99.0% or more chemically pure 1,3-BG has a chiral purity of 99.1% or more (e.g., R-enantiomer). In some embodiments, the 99.0% or more chemically pure 1,3-BG has a chiral purity of 99.2% or more (e.g., R-enantiomer). In some embodiments, the 99.0% or more chemically pure 1,3-BG has a chiral purity of 99.3% or more (e.g., R-enantiomer). In some embodiments, the 99.0% or more chemically pure 1,3-BG has a chiral purity of 99.4% or more (e.g., R-enantiomer). In some embodiments, the 99.0% or more chemically pure 1,3-BG has a chiral purity of 99.5% or more (e.g., R-enantiomer). In some embodiments, the 99.0% or more chemically pure 1,3-BG has a chiral purity of 99.6% or more (e.g., R-enantiomer). In some embodiments, the 99.0% or more chemically pure 1,3-BG has a chiral purity of 99.7% or more (e.g., R-enantiomer). In some embodiments, the 99.0% or more chemically pure 1,3-BG has a chiral purity of 99.8% or more (e.g., R-enantiomer). In some embodiments, the 99.0% or more chemically pure 1,3-BG has a chiral purity of 99.9% or more (e.g., R-enantiomer). In some embodiments, the 99.0% or more chemically pure 1,3-BG has essentially only R-enantiomer, and the S-enantiomer is not detectable, e.g., by GC-MS or LC-MS. In other embodiments, the 99.0% or more chemically pure 1,3-BG is enriched in R-enantiomer, e.g., includes 45% or less S-enantiomer, 40% or less S-enantiomer, 35% or less S-enantiomer, 30% or less S-enantiomer, 25% or less S-enantiomer, 20% or less S-enantiomer, 15% or less S-enantiomer, 10% or less S-enantiomer, or 5% or less S-enantiomer.

In some embodiments, the 99.0% or more chemically pure 1,3-BG has a chiral purity of 95% or more (e.g., 96% or more, 97% or more, 98% or more, 99.0% or more, 99.1% or more, 99.2% or more; e.g., R-enantiomer), and between 1 ppm and 1000 ppm of one or both of 3-hydroxy-butanal and 4-hydroxy-2-butanone (e.g., between 1 ppm and 900 ppm, between 1 ppm and 800 ppm, between 1 ppm and 700 ppm, between 1 ppm and 600 ppm, between 1 ppm and 500 ppm, between 1 ppm and 400 ppm, between 1 and 300 ppm, between 1 and 200 ppm, between 1 and 100 ppm, between 1 and 90 ppm, between 1 and 80 ppm, between 1 and 70 ppm, between 1 and 60 ppm, between 1 and 50 ppm, between 1 and 40 ppm, between 1 and 30 ppm, between 1 and 20 ppm, or between 1 and 10 ppm. In some embodiments, the 99.0% or more chemically pure 1,3-BG has a chiral purity of 95% or more (e.g., 96% or more, 97% or more, 98% or more, 99.0% or more, 99.1 or more, 99.2% or more), and between 1 ppm and 400 ppm of one or both of 3-hydroxy-butanal and 4-hydroxy-2-butanone. In some embodiments, the 99.0% or more chemically pure 1,3-BG has a chiral purity of 95% or more (e.g., 96% or more, 97% or more, 98% or more, 99.0% or more, 99.1 or more, 99.2% or more), and between 1 ppm and less than 400 ppm of one or both of 3-hydroxy-butanal and 4-hydroxy-2-butanone.

In some embodiments, the 99.0% or more chemically pure 1,3-BG has a chiral purity of 55% or more (e.g., 60% or more, 70% or more, 75% or more, 80% or more, 85% or more, 90% or more; e.g., R-enantiomer), and between 1 ppm and 1000 ppm of one or both of 3-hydroxy-butanal and 4-hydroxy-2-butanone (e.g., between 1 ppm and 900 ppm, between 1 ppm and 800 ppm, between 1 ppm and 700 ppm, between 1 ppm and 600 ppm, between 1 ppm and 500 ppm, between 1 ppm and 400 ppm, between 1 and 300 ppm, between 1 and 200 ppm, between 1 and 100 ppm, between 1 and 90 ppm, between 1 and 80 ppm, between 1 and 70 ppm, between 1 and 60 ppm, between 1 and 50 ppm, between 1 and 40 ppm, between 1 and 30 ppm, between 1 and 20 ppm, or between 1 and 10 ppm. In some embodiments, the 99.0% or more chemically pure 1,3-BG has a chiral purity of 55% or more (e.g., 60% or more, 70% or more, 75% or more, 80% or more, 85% or more, 90% or more; e.g., R-enantiomer), and between 1 ppm and 400 ppm of one or both of 3-hydroxy-butanal and 4-hydroxy-2-butanone. In some embodiments, the 99.0% or more chemically pure 1,3-BG has a chiral purity of 55% or more (e.g., 60% or more, 70% or more, 75% or more, 80% or more, 85% or more, 90% or more; e.g., R-enantiomer), and between 1 ppm and less than 400 ppm of one or both of 3-hydroxy-butanal and 4-hydroxy-2-butanone.

In some embodiments, the bioderived 1,3-BG has a higher purity level (e.g., chemical or chiral purity, or both chemical and chiral purity) than industrial-grade or cosmetic-grade bio-BG. In some embodiments, the bioderived 1,3-BG has about the same purity level as industrial-grade or cosmetic-grade bio-BG (e.g., a purity level of ±0.5%). In some embodiments, the bioderived 1,3-BG has a lower purity level than industrial-grade or cosmetic-grade bio-BG.

In some embodiments, the bioderived 1,3-BG has a higher purity (e.g., chemical or chiral purity, or both chemical and chiral purity) than industrial-grade or cosmetic-grade petro-BG. In some embodiments, the bioderived 1,3-BG has about the same purity level as industrial-grade or cosmetic grade petro-BG (e.g., a purity level of ±0.5%). In some embodiments, the bioderived 1,3-BG has a lower purity level than industrial-grade or cosmetic-grade petro-BG.

In some embodiments, the bioderived 1,3-BG has more R-enantiomer than S-enantiomer, and thus is enriched in R-enantiomer. In some embodiments, the bioderived 1,3-BG with the higher levels of R-enantiomer than S-enantiomer has a chiral purity level of at least 55%, at least 60%, at least 65%, at least 70%, at least 75%, at least 80%, at least 85%, at least 90%, at least 95%, or at least 99%, e.g., on a weight/weight basis. In some embodiments, the bioderived 1,3-BG with the higher levels of R-enantiomer than S-enantiomer has a chiral purity level of at least 95%, at least 96%, at least 97%, at least 98%, or at least 99%. In some embodiments, the bioderived 1,3-BG with the higher levels of R-enantiomer than S-enantiomer has a chiral purity level of at least 99.0%, at least 99.1%, at least 99.2%, at least 99.3%, at least 99.4%, at least 99.5%, at least 99.6%, at least 99.7%, at least 99.8%, or at least 99.9%.

In some embodiments, the bioderived 1,3-BG has more S-enantiomer than R-enantiomer, and thus is enriched in the S-enantiomer. In some embodiments, the bioderived 1,3-BG with the higher levels of S-enantiomer than R-enantiomer has a chiral purity level of at least 55%, at least 60%, at least 65%, at least 70%, at least 75%, at least 80%, at least 85%, at least 90%, at least 95%, or at least 99%, e.g., on a weight/weight basis. In some embodiments, the bioderived 1,3-BG with the higher levels of S-enantiomer than R-enantiomer has a chiral purity level of at least 95%, at least 96%, at least 97%, at least 98%, or at least 99%. In some embodiments, the bioderived 1,3-BG with the higher levels of S-enantiomer than R-enantiomer has a chiral purity level of at least 99.0%, at least 99.1%, at least 99.2%, at least 99.3%, at least 99.4%, at least 99.5%, at least 99.6%, at least 99.7%, at least 99.8%, or at least 99.9%.

In some embodiments, the bioderived 1,3-BG has a higher chiral purity level (e.g., higher level of R-enantiomer) than industrial grade or cosmetic grade bio-BG. In some embodiments, the bioderived 1,3-BG has about the same chiral purity level (e.g., R-enantiomer level) as industrial-grade or cosmetic grade-bio-BG (e.g., a chiral purity level of ±0.5%, e.g., of R-enantiomer levels).

In some embodiments, the bioderived 1,3-BG has a higher chiral purity level (e.g., higher level of R-enantiomer) than industrial-grade or cosmetic-grade petro-BG. In some embodiments, the bioderived 1,3-BG has about the same chiral purity level (e.g., higher level of R-enantiomer) as industrial-grade or cosmetic-grade petro-BG (e.g., a purity level of ±0.5%).

In some embodiments, the bioderived 1,3-BG has detectable levels of one or more contaminants that are not detectable in petro-BG or that are present at higher levels or at lower levels in bioderived 1,3-BG relative to petro-BG (e.g., industrial grade or cosmetic grade). In some embodiments, the contaminant levels in bioderived 1,3-BG are detectable by sensory analysis, e.g., conducted by a trained individual. In some embodiments, the contaminant levels are detectable in bioderived by their relative signal intensity in a GC-MS chromatogram or an LC-MS chromatogram (e.g., total ion current (TIC), extracted ion current (XIC)). In some embodiments, the bioderived 1,3-BG has detectable levels of one or more contaminants that are not detectable in industrial petro-BG or that are present at higher levels or at lower levels in bioderived 1,3-BG compared to industrial grade petro-BG. In some embodiments, the bioderived 1,3-BG has detectable levels of one or more contaminants that are not detectable in cosmetic grade petro-BG or that are present at higher levels or at lower levels in bio-BG compared to cosmetic grade petro-BG.

In some embodiments, the bioderived 1,3-BG has detectable levels of two or more, three or more, four or more, five or more, six or more, seven or more, eight or more, nine or more, or ten or more contaminants that are not detectable in petro-BG (e.g. cosmetic-grade or industrial grade petro-BG) or that are present at higher levels in bioderived 1,3-BG compared to petro-BG.

In some embodiments, the bioderived 1,3-BG has detectable levels of two or more, three or more, four or more, five or more, six or more, seven or more, eight or more, nine or more, or ten or more contaminants that are present at lower levels in bioderived 1,3-BG compared to petro-BG.

In some embodiments, the bioderived 1,3-BG has levels of one or more contaminants that are present at concentrations (e.g., in weight/weight percent) that are at least 2-fold, at least 3-fold, at least 4-fold, at least 5-fold, at least 6-fold, at least 7-fold, at least 8-fold, at least 9-fold, at least 10-fold, at least 12-fold, at least 15-fold, at least 20-fold, at least 30-fold, at least 40-fold, at least 50-fold, at least 60-fold, at least 70-fold, at least 80-fold, at least 90-fold, at least 100-fold, at least 150-fold, at least 200-fold, at least 300-fold, at least 400-fold, at least 500-fold, at least 600-fold, at least 700-fold, at least 800-fold, at least 900-fold, or at least 1,000-fold higher than the concentrations of the contaminant in petro-BG (e.g., industrial-grade or cosmetic grade petro-BG).

In some embodiments, the bioderived 1,3-BG has levels of one or more contaminants that are present at concentrations (e.g., in weight/weight percent) that are at least 2-fold, at least 3-fold, at least 4-fold, at least 5-fold, at least 6-fold, at least 7-fold, at least 8-fold, at least 9-fold, at least 10-fold, at least 12-fold, at least 15-fold, at least 20-fold, at least 30-fold, at least 40-fold, at least 50-fold, at least 60-fold, at least 70-fold, at least 80-fold, at least 90-fold, at least 100-fold, at least 150-fold, at least 200-fold, at least 300-fold, at least 400-fold, at least 500-fold, at least 600-fold, at least 700-fold, at least 800-fold, at least 900-fold, or at least 1,000-fold lower than the concentrations of the contaminant in petro-BG (e.g., industrial-grade or cosmetic grade petro-BG).

In some embodiments, the levels of a contaminant detectable in bioderived 1,3-BG that is not detectable in petro-BG or present at higher levels in bioderived 1,3-BG relative to petro-BG are less than 10,000 ppm, less than 9,000 ppm, less than 8,000 ppm, less than 7,000 ppm, less than 6,000 ppm, less than 5,000 ppm, less than 4,000 ppm, less than 3,000 ppm, less than 2,000 ppm, less than 1,500 ppm, less than 1,000 ppm, less than 900 ppm, less than 800 ppm, less than 700 ppm, less than 600 ppm, less than 500 ppm, less than 400 ppm, less than 300 ppm, less than 200 ppm, less than 100 ppm, less than 50 ppm, or less than 25 ppm in bioderived 1,3-BG.

In some embodiments, the levels of a contaminant detectable in bioderived 1,3-BG that is present at lower levels in bioderived 1,3-BG relative to petro-BG are less than 10,000 ppm, less than 9,000 ppm, less than 8,000 ppm, less than 7,000 ppm, less than 6,000 ppm, less than 5,000 ppm, less than 4,000 ppm, less than 3,000 ppm, less than 2,000 ppm, less than 1,500 ppm, less than 1,000 ppm, less than 900 ppm, less than 800 ppm, less than 700 ppm, less than 600 ppm, less than 500 ppm, less than 400 ppm, less than 300 ppm, less than 200 ppm, less than 100 ppm, less than 50 ppm, or less than 25 ppm in bioderived 1,3-BG.

In some embodiments, the levels of a contaminant detectable in bioderived 1,3-BG that is not detectable in petro-BG or present at higher levels in bioderived 1,3-BG relative to petro-BG are 25 ppm or more, 50 ppm or more, 100 ppm or more, 200 ppm or more, 300 ppm or more, 400 ppm or more, 500 ppm or more, 600 ppm or more, 700 ppm or more, 800 ppm or more, 900 ppm or more, 1,000 ppm or more, 1,500 ppm or more, 2,000 ppm or more, 3,000 ppm or more, 4,000 ppm or more, 5,000 ppm or more, 6,000 ppm or more, 7,000 ppm or more, 8,000 ppm or more, 9,000 ppm or more, 10,000 ppm or more in bioderived 1,3-BG.

In some embodiments, the levels of a contaminant detectable in bioderived 1,3-BG that is present at lower levels in bioderived 1,3-BG relative to petro-BG are 25 ppm or more, 50 ppm or more, 100 ppm or more, 200 ppm or more, 300 ppm or more, 400 ppm or more, 500 ppm or more, 600 ppm or more, 700 ppm or more, 800 ppm or more, 900 ppm or more, 1,000 ppm or more, 1,500 ppm or more, 2,000 ppm or more, 3,000 ppm or more, 4,000 ppm or more, 5,000 ppm or more, 6,000 ppm or more, 7,000 ppm or more, 8,000 ppm or more, 9,000 ppm or more, 10,000 ppm or more in bioderived 1,3-BG.

In some embodiments, the levels of a contaminant detectable in bioderived 1,3-BG that is not detectable in petro-BG or present at higher levels in bioderived 1,3-BG relative to petro-BG are less than 25 ppm, less than 50 ppm, less than 100 ppm, less than 90 ppm, less than 80 ppm, less than 70 ppm, less than 60 ppm, less than 50 ppm, less than 40 ppm, less than 30 ppm, less than 20 ppm, less than 10 ppm, or undetectable levels in petro-BG (e.g., cosmetic grade or industrial grade).

In some embodiments, the levels of a contaminant detectable in bioderived 1,3-BG that is present at lower levels in bioderived 1,3-BG relative to petro-BG are less than 25 ppm, less than 50 ppm, less than 100 ppm, less than 90 ppm, less than 80 ppm, less than 70 ppm, less than 60 ppm, less than 50 ppm, less than 40 ppm, less than 30 ppm, less than 20 ppm, less than 10 ppm, or undetectable levels in petro-BG (e.g., cosmetic grade or industrial grade).

In some embodiments, a contaminant is present in bioderived 1,3-BG at levels of 25 ppm or more (e.g., 25 ppm or more, 50 ppm or more, 100 ppm, 200 ppm or more, 300 ppm or more, 400 ppm or more, 500 ppm or more, 600 ppm or more, 700 ppm or more, 800 ppm or more, 900 ppm or more, 1,000 ppm or more, 1,500 ppm or more, 2,000 ppm or more, 3,000 ppm or more, 4,000 ppm or more, 5,000 ppm or more, 6,000 ppm or more, 7,000 ppm or more, 8,000 ppm or more, 9,000 ppm or more, or 10,000 ppm or more) and the contaminant is present in petro-BG (e.g., industrial grade or cosmetic grade) at levels of less than 100 ppm, less than 90 ppm, less than 80 ppm, less than 70 ppm, less than 60 ppm, less than 50 ppm, less than 40 ppm, less than 30 ppm, less than 20 ppm, less than 10 ppm, or at undetectable levels.

In some embodiments, a contaminant is present in petro-BG at levels of 25 ppm or more (e.g., 25 ppm or more, 50 ppm or more, 100 ppm, 200 ppm or more, 300 ppm or more, 400 ppm or more, 500 ppm or more, 600 ppm or more, 700 ppm or more, 800 ppm or more, 900 ppm or more, 1,000 ppm or more, 1,500 ppm or more, 2,000 ppm or more, 3,000 ppm or more, 4,000 ppm or more, 5,000 ppm or more, 6,000 ppm or more, 7,000 ppm or more, 8,000 ppm or more, 9,000 ppm or more, or 10,000 ppm or more) and the contaminant is present in bioderived 1,3-BG (e.g., industrial grade or cosmetic grade) at levels of less than 100 ppm, less than 90 ppm, less than 80 ppm, less than 70 ppm, less than 60 ppm, less than 50 ppm, less than 40 ppm, less than 30 ppm, less than 20 ppm, less than 10 ppm, or at undetectable levels.

In some embodiments, the levels of a contaminant in bioderived 1,3-BG that is not detectable in petro-BG or present at higher levels in bioderived 1,3-BG relative to petro-BG are less than 10,000 ppm, less than 9,000 ppm, less than 8,000 ppm, less than 7,000 ppm, less than 6,000 ppm, less than 5,000 ppm, less than 4,000 ppm, less than 3,000 ppm, less than 2,000 ppm, less than 1,500 ppm, less than 1,000 ppm, less than 900 ppm, less than 800 ppm, less than 700 ppm, less than 600 ppm, less than 500 ppm, less than 400 ppm, less than 300 ppm, less than 200 ppm, less than 100 ppm, less than 50 ppm, or less than 25 ppm.

In some embodiments, the levels of a contaminant in bioderived 1,3-BG that is present at lower levels in bioderived 1,3-BG relative to petro-BG are less than 10,000 ppm, less than 9,000 ppm, less than 8,000 ppm, less than 7,000 ppm, less than 6,000 ppm, less than 5,000 ppm, less than 4,000 ppm, less than 3,000 ppm, less than 2,000 ppm, less than 1,500 ppm, less than 1,000 ppm, less than 900 ppm, less than 800 ppm, less than 700 ppm, less than 600 ppm, less than 500 ppm, less than 400 ppm, less than 300 ppm, less than 200 ppm, less than 100 ppm, less than 50 ppm, or less than 25 ppm.

In some embodiments, the contaminants detectable in bioderived 1,3-BG that are not detectable in petro-BG (e.g., industrial grade or cosmetic grade) or present at higher levels in bioderived 1,3-BG relative to petro-BG, or present at lower levels in bioderived 1,3-BG relative to petro-BG can include 3-hydroxy-butanal, 4-hydroxy-2-butanone, 4-(3-hydroxybutoxy)butan-2-one (proposed structure also referred to herein as 3-hydroxy-butyl-3-oxo-butane ether (proposed structure) or “Compound 7”; see also Table 5) and 4-((4-hydroxybutan-2-yl)oxy)-butan-2-one (proposed structure also referred to herein as 2-methyl-3-hydroxy-propyl-3-oxo-butane ether (proposed structure) or “Compound 9”; see also Table 5), or combinations thereof.

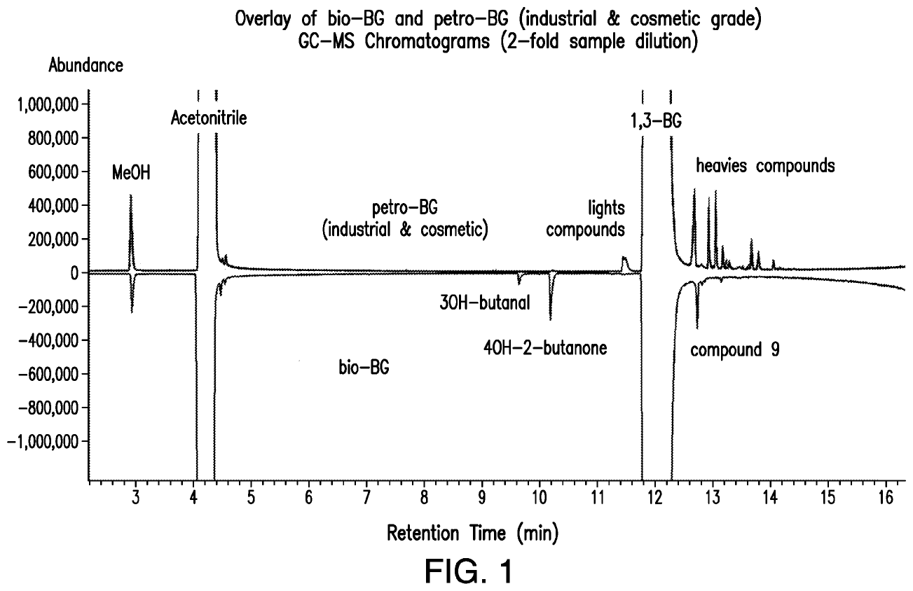

In some embodiments, the contaminants detectable in bioderived 1,3-BG that are not detectable in petro-BG (e.g., industrial grade or cosmetic grade) or present at higher levels in bioderived 1,3-BG relative to petro-BG, or present at lower levels in bioderived 1,3-BG relative to petro-BG can include 3-hydroxy-butanal. See, e.g., FIG. 1 and FIG. 2. In some embodiments, the bioderived 1,3-BG has 3-hydroxy-butanal levels of less than 1,000 ppm, less than 900 ppm, less than 800 ppm, less than 700 ppm, less than 600 ppm, less than 500 ppm, less than 400 ppm, less than 300 ppm, less than 200 ppm, less than 100 ppm, less than 50 ppm, or less than 25 ppm. In some embodiments, the bioderived 1,3-BG has 3-hydroxy-butanal levels of 100 ppm or more, 200 ppm or more, 300 ppm or more, 400 ppm or more, 500 ppm or more, 600 ppm or more, 700 ppm or more, 800 ppm or more, 900 ppm or more, or 1,000 ppm or more.

In some embodiments, the contaminants detectable in bioderived 1,3-BG that are not detectable in petro-BG (e.g., industrial grade or cosmetic grade) or present at higher levels in bioderived 1,3-BG relative to petro-BG, or present at lower levels in bioderived 1,3-BG relative to petro-BG can include 4-hydroxy-2-butanone. See, e.g., FIG. 1 and FIG. 2. In some embodiments, the bioderived 1,3-BG has 4-hydroxy-2-butanone levels of less than 1,000 ppm, less than 900 ppm, less than 800 ppm, less than 700 ppm, less than 600 ppm, less than 500 ppm, less than 400 ppm, less than 300 ppm, less than 200 ppm, less than 100 ppm, less than 50 ppm, or less than 25 ppm. In some embodiments, the bioderived 1,3-BG has 4-hydroxy-2-butanone levels of 25 ppm or more, 50 ppm or more, 100 ppm or more, 200 ppm or more, 300 ppm or more, 400 ppm or more, 500 ppm or more, 600 ppm or more, 700 ppm or more, 800 ppm or more, 900 ppm or more, or 1,000 ppm or more.

In some embodiments, the contaminants detectable in bioderived 1,3-BG that are not detectable in petro-BG (e.g., industrial grade or cosmetic grade) or present at higher levels in bioderived 1,3-BG relative to petro-BG, or present at lower levels in bioderived 1,3-BG relative to petro-BG can include Compound 7. See, e.g., FIG. 2. In some embodiments, the bioderived 1,3-BG has Compound 7 levels of less than 2,000 ppm, less than 1,500 ppm, less than 1,000 ppm, less than 900 ppm, less than 800 ppm, less than 700 ppm, less than 600 ppm, less than 500 ppm, less than 400 ppm, less than 300 ppm, less than 200 ppm, less than 100 ppm, less than 50 ppm, or less than 25 ppm. In some embodiments, the bioderived 1,3-BG has Compound 7 levels of 25 ppm or more, 50 ppm or more, 100 ppm or more, 200 ppm or more, 300 ppm or more, 400 ppm or more, 500 ppm or more, 600 ppm or more, 700 ppm or more, 800 ppm or more, 900 ppm or more, 1,000 ppm or more, 1,500 ppm or more, or 2,000 ppm or more.

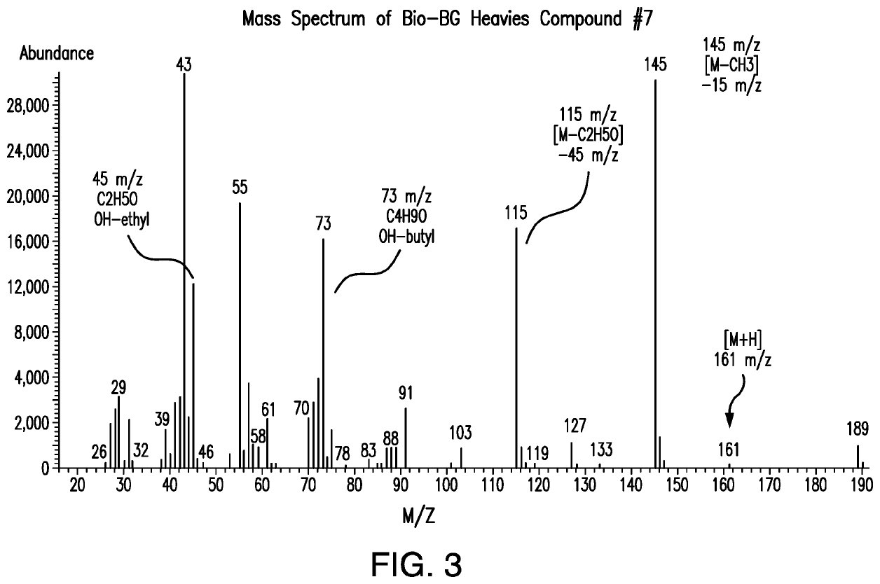

In some embodiments, the contaminants detectable in bioderived 1,3-BG that are not detectable in petro-BG (e.g., industrial grade or cosmetic grade) or present at higher levels in bioderived 1,3-BG relative to petro-BG, or present at lower levels in bioderived 1,3-BG relative to petro-BG can include a compound characterized by a mass spectrum according to FIG. 3. In FIG. 3, the proposed interpretations of certain mass fragments are not intended to be limiting.

In some embodiments, the contaminants detectable in bioderived 1,3-BG that are not detectable in petro-BG (e.g., industrial grade or cosmetic grade) or present at higher levels in bioderived 1,3-BG relative to petro-BG, or present at lower concentrations in bioderived 1,3-BG relative to petro-BG are detectable in a GC-MS chromatogram as a peak (e.g., total ion current (TIC)) eluting with a relative retention time of between 0.97-0.99 (e.g., 0.97; 0.98; 0.99) when taking the relative retention time of 1,3-BG as 1.0. See, e.g., FIG. 2 (RT Compound 7=12.05 min; RT 1,3-BG=11.85 min; see also Table 5).

In some embodiments, the contaminants detectable in bioderived 1,3-BG that are not detectable in petro-BG (e.g., industrial grade or cosmetic grade) or present at higher levels in bioderived 1,3-BG relative to petro-BG, or present at lower levels in bioderived 1,3-BG relative to petro-BG can include Compound 9. See, e.g., FIG. 2. In some embodiments, the bioderived 1,3-BG has Compound 9 levels of less than 1,500 ppm, less than 1,000 ppm, less than 900 ppm, less than 800 ppm, less than 700 ppm, less than 600 ppm, less than 500 ppm, less than 400 ppm, less than 300 ppm, less than 200 ppm, less than 100 ppm, less than 50 ppm, or less than 25 ppm. In some embodiments, the bioderived 1,3-BG has Compound 9 levels of 25 ppm or more, 50 ppm or more, 100 ppm or more, 200 ppm or more, 300 ppm or more, 400 ppm or more, 500 ppm or more, 600 ppm or more, 700 ppm or more, 800 ppm or more, 900 ppm or more, 1,000 ppm or more, or 1,500 ppm or more.

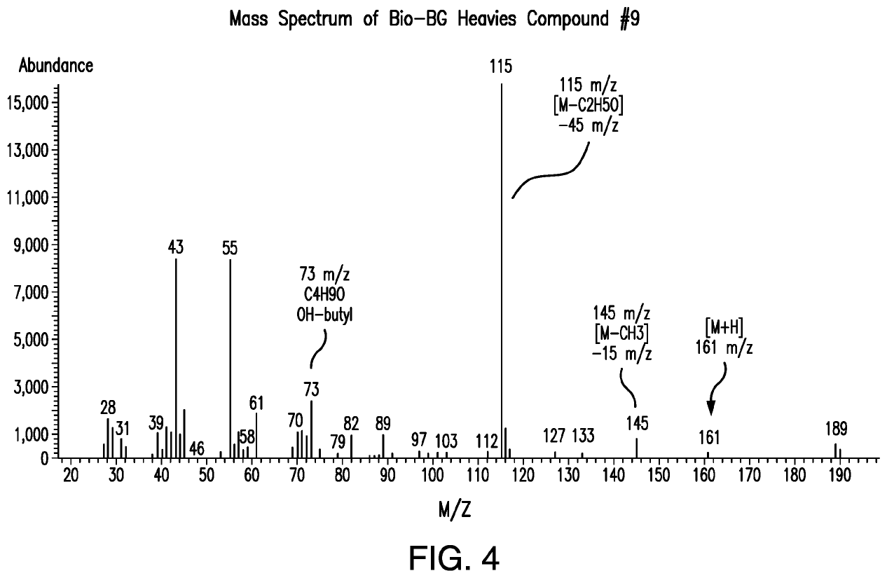

In some embodiments, the contaminants detectable in bioderived 1,3-BG that are not detectable in petro-BG (e.g., industrial grade or cosmetic grade) or present at higher levels in bioderived 1,3-BG relative to petro-BG, or present at lower levels in bioderived 1,3-BG relative to petro-BG can include a compound characterized by a mass spectrum according to FIG. 4. In FIG. 4, the proposed interpretations of certain mass fragments are not intended to be limiting.

In some embodiments, the contaminants detectable in bioderived 1,3-BG that are not detectable in petro-BG (e.g., industrial grade or cosmetic grade) or present at higher levels in bioderived 1,3-BG relative to petro-BG, or present at lower levels in bioderived 1,3-BG relative to petro-BG are detectable in a GC-MS chromatogram as a peak (e.g., total ion current (TIC)) eluting with a relative retention time of between 0.94-0.96 (e.g., 0.94; 0.95; 0.96) when taking the relative retention time of 1,3-BG as 1.0. See, e.g., FIG. 2 (RT Compound 9=12.51 min; RT 1,3-BG=11.85 min; see also Table 5).

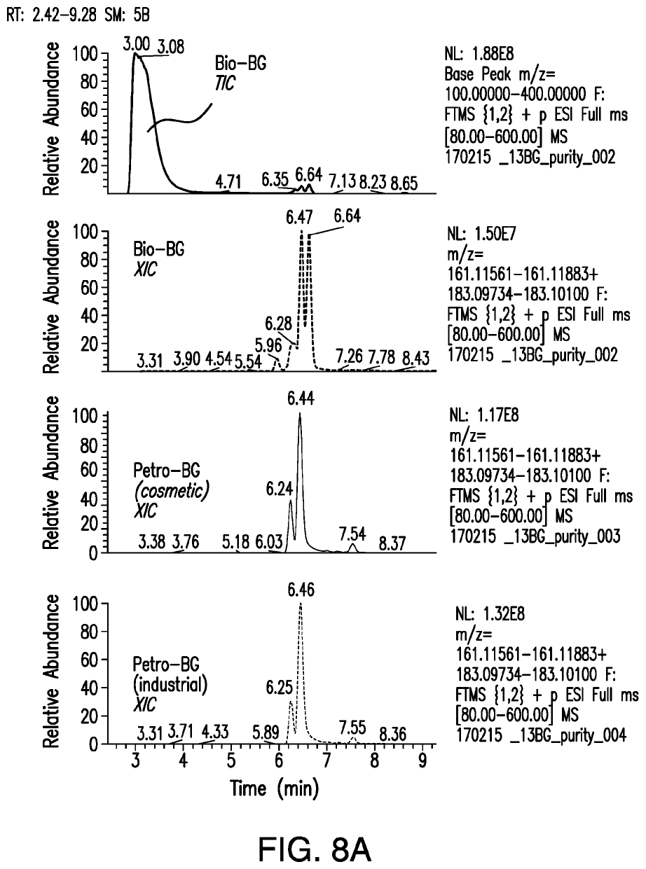

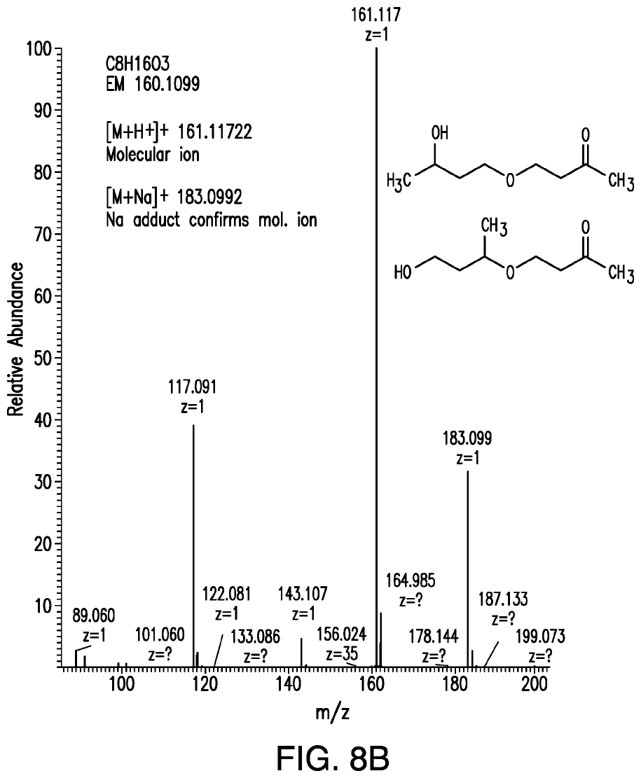

In some embodiments, the contaminants detectable in bioderived 1,3-BG that are not detectable in petro-BG (e.g., industrial grade or cosmetic grade) or present at higher levels in bioderived 1,3-BG relative to petro-BG, or present at lower levels in bioderived 1,3-BG relative to petro-BG are detectable in an LC-MS chromatogram (e.g., extracted ion current (XIC) eluting with a relative retention time of between 0.45-0.55 (e.g., 0.94; 0.95; 0.96) when taking the relative retention time of 1,3-BG as 1.0. See, e.g., FIG. 8A (RT compound-=6.0 min-6.7 min; RT 1,3-BG=3.08 min; see also Table 5).

In some embodiments, the contaminants detectable in bioderived 1,3-BG that are not detectable in petro-BG (e.g., industrial grade or cosmetic grade) or present at higher levels in bioderived 1,3-BG relative to petro-BG, or present at lower levels in bioderived 1,3-BG relative to petro-BG have an elemental composition of C₈H₁₆O₃ and a molecular weight of 160. See, e.g., FIG. 8B. In FIG. 8B, the proposed interpretations of certain mass fragments are not intended to be limiting.

In some embodiments, the contaminants detectable in bioderived 1,3-BG that are not detectable in petro-BG (e.g., industrial grade or cosmetic grade) or present at higher levels in bioderived 1,3-BG relative to petro-BG are characterized by a mass spectrum according to FIG. 8B.

In some embodiments, fewer “heavies” contaminants are detectable by GC-MS in bioderived 1,3-BG provided herein relative to petro-BG (e.g., industrial grade or cosmetic grade), whereas the “heavies” contaminants are eluting with a relative retention time of between 0.8-0.95 when taking the relative retention time of 1,3-BG as 1.0. See, e.g., FIG. 2.

In some embodiments, the bioderived 1,3-BG has an overall lower level of “heavies” contaminants than petro-BG (e.g., industrial grade or cosmetic grade). In some embodiments, the bioderived 1,3-BG has an overall lower level of “lights” contaminants than petro-BG. In some embodiments, the bioderived 1,3-BG has overall lower levels of “heavies” and “lights” contaminants than petro-BG. In some embodiments, the overall purity of bioderived 1,3-BG is 99% or higher (e.g., 99.0%, 99.1%, 99.2%, 99.3%, 99.4%, 99.5%, 99.6%, 99.7%, 99.8%, 99.9%, or higher) and the overall level of heavies contaminants is 1.0% or less (e.g., 1.0%, 0.9%, 0.8%, 0.7%, 0.6%, 0.5%, 0.4%, 0.3%, 0.2%, 0.1% or less). In some embodiments, the overall purity of bioderived 1,3-BG is 99% or higher (e.g., 99.0%, 99.1%, 99.2%, 99.3%, 99.4%, 99.5%, 99.6%, 99.7%, 99.8%, 99.9%, or higher) and the overall level of lights contaminants is 1.0% or less (e.g., 1.0%, 0.9%, 0.8%, 0.7%, 0.6%, 0.5%, 0.4%, 0.3%, 0.2%, 0.1% or less). In some embodiments, the overall purity of bioderived 1,3-BG is 99% or higher (e.g., 99.0%, 99.1%, 99.2%, 99.3%, 99.4%, 99.5%, 99.6%, 99.7%, 99.8%, 99.9%, or higher), the overall level of heavies contaminants is 0.8% or less (e.g., 0.8%, 0.7%, 0.6%, 0.5%, 0.4%, 0.3%, 0.2%, 0.1% or less), and the overall level of lights contaminants is 0.2% or less (e.g., 0.2%, 0.1%, 0.0%). See, e.g., Table 3.

Preferably, in all embodiments herein the lights and heavies impurities present in bio-BG are at lower overall levels, and alternatively, lower individual levels, than those in industrial or cosmetic grade petro-BG.

In some embodiments, the overall chiral purity (e.g., R-enantiomer level) of bioderived 1,3-BG is 55% or higher (e.g., 60%, 65%, 70%, 75%, 80%, 85%, 90%, 95%, or higher). In some embodiments, the overall chiral purity (e.g., R-enantiomer level) of bioderived 1,3-BG is 99% or higher (e.g., 99.0%, 99.1%, 99.2%, 99.3%, 99.4%, 99.5%, 99.6%, 99.7%, 99.8%, 99.9%, or higher), preferably 99.5% or higher. In preferred embodiments, the overall chiral purity (e.g., R-enantiomer level) of bioderived 1,3-BG is 99% or higher (e.g., 99.0%, 99.1%, 99.2%, 99.3%, 99.4%, 99.5%, 99.6%, 99.7%, 99.8%, 99.9%, or higher), preferably 99.5% or higher, and the overall chemical purity of bioderived 1,3-BG is 99% or higher (e.g., 99.0%, 99.1%, 99.2%, 99.3%, 99.4%, 99.5%, 99.6%, 99.7%, 99.8%, 99.9%, or higher). In some embodiments, the overall chiral purity (e.g., R-enantiomer level) of bioderived 1,3-BG is 55% or higher (e.g., 60%, 65%, 70%, 75%, 80%, 85%, 90%, 95%, or higher), and the overall chemical purity of bioderived 1,3-BG is 99% or higher (e.g., 99.0%, 99.1%, 99.2%, 99.3%, 99.4%, 99.5%, 99.6%, 99.7%, 99.8%, 99.9%, or higher).

In some embodiments, the bioderived 1,3-BG has an UV absorbance between 220 nm and 260 nm that is at least at least 2-fold, at least 3-fold, at least 4-fold, at least 5-fold, at least 6-fold, at least 7-fold, at least 8-fold, at least 9-fold, or at least 10-fold lower than the UV absorbance of petro-BG (e.g., cosmetic grade or industrial grade).

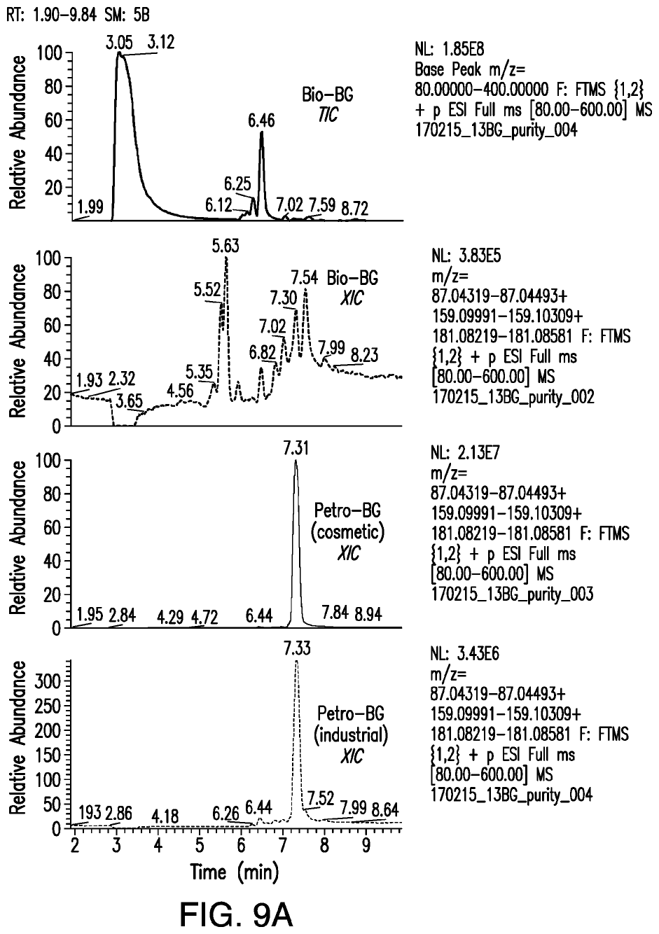

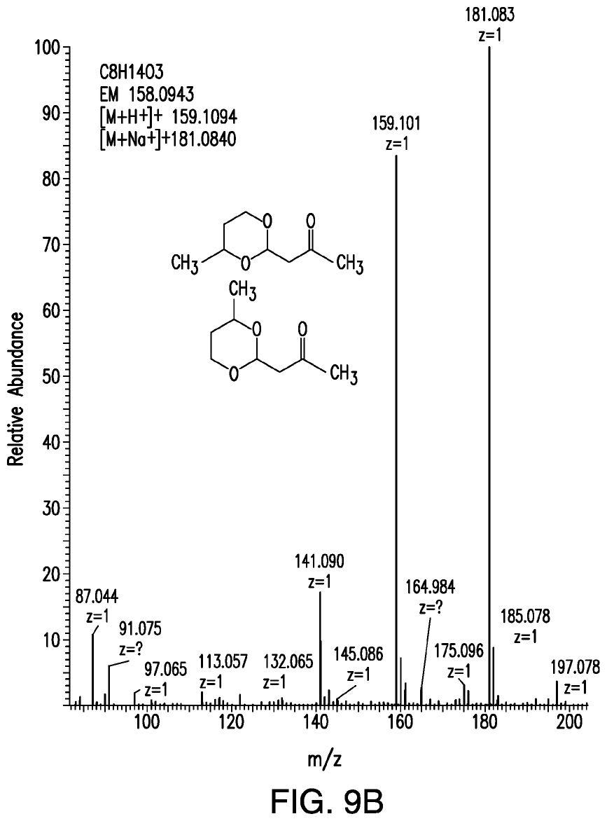

In some embodiments, the bioderived 1,3-BG does not have detectable levels of 1-4-(4-methyl-1,3-dioxan-2-yl)propan-2-one, or has lower levels of 1-4-(4-methyl-1,3-dioxan-2-yl)propan-2-one than petro-BG, e.g., as determined by LC-MS (e.g., extracted ion current (XIC)). See, e.g., Table 6, FIG. 9A, FIG. 9B. In FIG. 9B, the proposed interpretations of certain mass fragments are not intended to be limiting.

In some embodiments, the bioderived 1,3-BG does not have detectable levels or has lower levels than petro-BG of a contaminant eluting in an LC-MS chromatogram with a relative retention time of between 0.40-0.43 when taking the relative retention time of 1,3-BG as 1.0. See, e.g., FIG. 9A (RT compounds=7.31 min-7.33 min; RT 1,3-BG=3.05 min; see also Table 6).

In some embodiments, the bioderived 1,3-BG does not have detectable levels or has lower levels than petro-BG of a contaminant having an elemental composition of C₈H₁₄₀₃ and a molecular weight of 158. See, e.g., FIG. 9B.

In some embodiments, the bioderived 1,3-BG does not have detectable levels or has lower levels than petro-BG of a contaminant characterized by a mass spectrum according to FIG. 9B.

In some embodiments, the levels of the contaminant not detectable in bioderived 1,3-BG or present at lower levels in bioderived 1,3-BG than petro-BG are at least 2-fold, at least 3-fold, at least 4-fold, at least 5-fold, at least 6-fold, at least 7-fold, at least 8-fold, at least 9-fold, or at least 10-fold lower in bioderived 1,3-BG than in petro-BG.

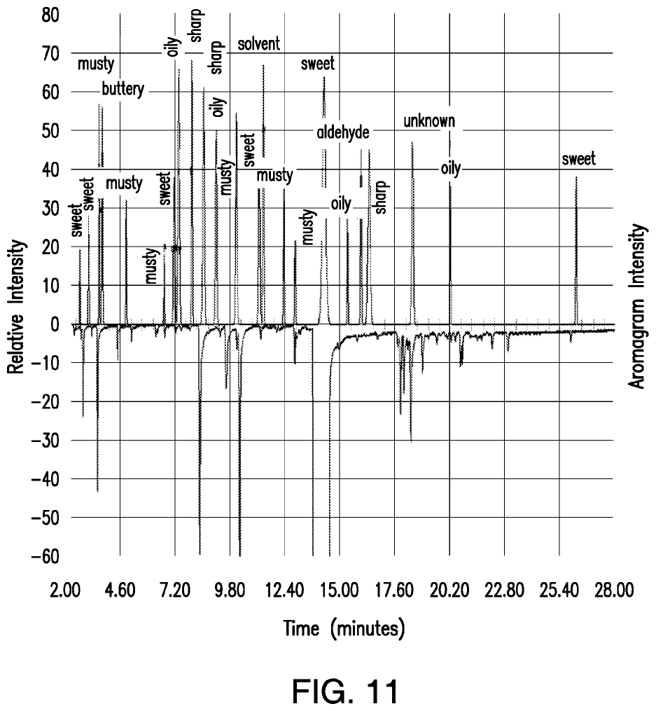

In some embodiments, the bioderived 1,3-BG has no detectable levels or only low levels of a compound found in cosmetic-grade petro-BG and characterized as having a “sharp,” “fecal,” “oily,” “sweet,” or “musty” odor, e.g., as determined by a sensory odor panel composed of trained individuals. See, e.g., Example 3. In some embodiments, the compound found in cosmetic-grade petro-BG corresponds to a compound identified in the GCMS-O analysis illustrated in FIG. 11 between 17.60 min and 25.40 min.

In some embodiments, the odor of bioderived 1,3-BG provided herein is rated as predominantly mildly sweet, oily, fruity, or a combination thereof, by a majority of members of a sensory odor panel.

In some embodiments, the odor of bioderived 1,3-BG provided herein is not rated as predominantly oily, paint-like, glue-like, or a combination thereof, by a majority of members of a sensory odor panel.

In some embodiments, fewer fractions with odor causing compounds are found in bioderived 1,3-BG by GC-MS analysis at retention times (RTs) longer than 1,3-BG than in cosmetic grade petro-BG. See, e.g., Example 3.

In some embodiments, cosmetic grade petro-BG includes GC fractions with sweet (e.g., 5 fractions or more), musty (e.g., 4 fractions or more), fruity (e.g., 1 fraction or more), oily (e.g., 3 fractions or more), citrus (e.g., 1 fraction or more), earthy (e.g., 1 fraction or more), aldehyde (e.g., 1 fraction or more), sharp (e.g., 1 fraction or more), or fecal (e.g., 1 fraction or more) odors, or combinations thereof.

In some embodiments, bioderived 1,3-BG includes GC fraction with sweet (e.g., 6 fractions or less), musty (e.g., 6 fractions or less), oily (e.g., 4 fractions or less), aldehyde (e.g., 1 fraction or less), sharp (e.g., 2 fraction or less), buttery (e.g., 1 fraction or less), solvent (e.g., 1 fraction or less) or unknown (e.g., 1 fraction or less) odors, or combinations thereof.

In some embodiments, bioderived 1,3-BG does not include a GC fraction with a fecal, an earthy, or a citrus odor, or combinations thereof.

In some embodiments, the bioderived 1,3-BG includes a GC fraction with a buttery or a solvent odor, or a combination thereof, that are not present in a cosmetic-grade petro-BG.

In some embodiments, the bioderived 1,3-BG includes a GC fraction with a fecal, a musty, or a sharp odor, or a combination thereof, having GC retention times longer than 1,3-BG.

In some embodiments, the bioderived 1,3-BG can have detectable levels of a compound such as acetaldehyde, 4-hydroxy-2-butanone, 3-buten-2-one (methyl vinyl ketone), diacetyl, 2-butenal (crotonaldehyde), 1-hydroxy-2-propanone, 3-hydroxy-2-butanone (acetoin), 3-hydroxy-butanal (3-hydroxy-butyraldehyde), 2,3-butanediol, 1,2-propanediol, 1,3-propanediol, 2-methyl-2-propyl-1,3-dioxepane, or combinations thereof. See also Tables 1 and 7. In some embodiments, the compound levels are detectable by olfactory analysis, e.g., by a trained individual. In some embodiments, the compound levels are detectable by the mass and relative signal intensity (e.g., total ion current (TIC)) in a GC-MS chromatogram. In some embodiments, the detectable levels of the compound are less than 1,000 ppm, less than 900 ppm, less than 800 ppm, less than 700 ppm, less than 600 ppm, less than 500 ppm, less than 400 ppm, less than 300 ppm, less than 200 ppm, less than 100 ppm, less than 90 ppm, less than 80 ppm, less than 70 ppm, less than 60 ppm, less than 50 ppm, less than 40 ppm, less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, or less than 1 ppm, e.g., as determined by coupled gas-chromatography-mass-spectrometry (GCMS). In some embodiments, the detectable levels of the compound are less than the odor threshold of the compound.

At least the following volatile compounds in bioderived 1,3-BG were detected only in gaseous headspace using absorbants. Without wishing to be bound by theory, the following exemplary compounds are thus believed to be present at a level of less than 1 ppm: acetaldehyde, 3-buten-2-one (methyl vinyl ketone), diacetyl, 2-butenal (crotonaldehyde), 3-hydroxy-2-butanone (acetoin), or combinations thereof. At least the following exemplary compounds can be unique to bioderived 1,3-BG: 4-hydroxy-2-butanone, diacetyl, 1-hydroxy-2-propanone, 2,3-butanediol, 1,2-propanediol, or 1,3-propanediol, or combinations thereof.

TABLE 1
Compounds identified in an exemplary bioderived 1,3-BG
product provided herein

| Compound | Structure |
|---|---|
| acetaldehyde | 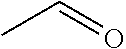 |
| 4-hydroxy-2-butanone (4OH-2- butanone) | 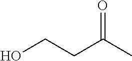 |
| 3-buten-2-one (methyl vinyl ketone, MVK) | 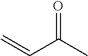 |
| diacetyl, 2-butenal (crotonaldehyde, Cr-Ald) | 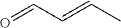 |
| 1-hydroxy-2-propanone | 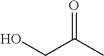 |
| 3-hydroxy-2-butanone (3OH-2- butanone) | 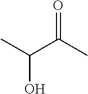 |
| 3-hydroxy-butanal | 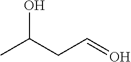 |
| 2,3-butanediol (2,3-BDO) | 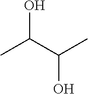 |
| 1,2-propanediol (1,2-PDO) | 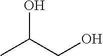 |
| 1,3-propanediol (1,3-PDO) | 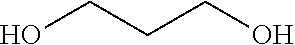 |
| 2-methyl-2-propyl-1,3-dioxepane | 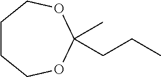 |

In some embodiments, the level of acetaldehyde, 4-hydroxy-2-butanone, 3-buten-2-one (methyl vinyl ketone), diacetyl, 2-butenal (crotonaldehyde), 1-hydroxy-2-propanone, 3-hydroxy-2-butanone (acetoin), 3-hydroxy-butanal (3-hydroxy-butyraldehyde), 2,3-butanediol, 1,2-propanediol, 1,3-propanediol, 1,3-dioxepane, 2-methyl-2-propyl, 2-methyl-2-propyl-1,3-dioxepane, or combinations thereof, is not detectable in the bioderived 1,3-BG, e.g., by GC-MS.

In some embodiments, the bioderived 1,3-BG has detectable levels of 3-hydroxy butanal or 4-hydroxy-2-butanone. In some embodiments, the bioderived 1,3-BG has less than 200 ppm, less than 100 ppm, less than 90 ppm, less than 80 ppm, less than 70 ppm, less than 60 ppm, less than 50 ppm, less than 40 ppm, less than 30 ppm, less than 20 ppm, or less than 10 ppm 3-hydroxy butanal or 4-hydroxy-2-butanone, e.g., as determined by GC-MS. In some embodiments, the bioderived 1,3-BG has less 3-hydroxy butanal or 4-hydroxy-2-butanone than the odor threshold of 3-hydroxy butanal or 4-hydroxy-2-butanone.

In some embodiments, the bioderived 1,3-BG has detectable levels of 3-hydroxy butanal. In some embodiments, the bioderived 1,3-BG has less than less than 200 ppm, less than 100 ppm, less than 90 ppm, less than 80 ppm, less than 70 ppm, less than 60 ppm, less than 50 ppm, less than 40 ppm, less than 30 ppm, less than 20 ppm, or less than 10 ppm 3-hydroxy butanal, e.g., as determined by GC-MS. In some embodiments, the level of 3-hydroxy butanal is not detectable in the bioderived 1,3-BG, e.g., by GCMS. In some embodiments, the bioderived 1,3-BG has less 3-hydroxy butanal than the odor threshold of 3-hydroxy butanal. In some embodiments, the bioderived 1,3-BG has less than 40 ppm 3-hydroxy-butanal.

In some embodiments, the bioderived 1,3-BG has detectable levels of 4-hydroxy-2-butanone. In some embodiments, the bioderived 1,3-BG has less than less than 200 ppm, less than 100 ppm, less than 90 ppm, less than 80 ppm, less than 70 ppm, less than 60 ppm, less than 50 ppm, less than 40 ppm, less than 30 ppm, less than 20 ppm, or less than 10 ppm 4-hydroxy-2-butanone, e.g., as determined by GC-MS. In some embodiments, the level of 4-hydroxy-2-butanone is not detectable in the bioderived 1,3-BG, e.g., by GC-MS. In some embodiments, the bioderived 1,3-BG has less 4-hydroxy-2-butanone than the odor threshold of 4-hydroxy-2-butanone.

In some embodiments, the bioderived 1,3-BG has detectable levels of 1-hydroxy-2-propanone. In some embodiments, the bioderived 1,3-BG has less than 100 ppm, less than 90 ppm, less than 80 ppm, less than 70 ppm, less than 60 ppm, less than 50 ppm, less than 40 ppm, less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, or less than 1 ppm 1-hydroxy-2-propanone, e.g., as determined by GC-MS. See, e.g., Table 1. In some embodiments, the level of 1-hydroxy-2-propanone is not detectable in the bioderived 1,3-BG, e.g., by GC-MS.

In some embodiments, the bioderived 1,3-BG has detectable levels of 1,2-propanediol. In some embodiments, the bioderived 1,3-BG has less than 100 ppm, less than 90 ppm, less than 80 ppm, less than 70 ppm, less than 60 ppm, less than 50 ppm, less than 40 ppm, less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, or less than 1 ppm 1,2-propanediol, e.g., as determined by GC-MS. See, e.g., Table 1. In some embodiments, the level of 1,2-propanediol is not detectable in the bioderived 1,3-BG, e.g., by GC-MS.

In some embodiments, the bioderived 1,3-BG has detectable levels of 1,3-propanediol. In some embodiments, the bioderived 1,3-BG has less than 200 ppm, less than 100 ppm, less than 90 ppm, less than 80 ppm, less than 70 ppm, less than 60 ppm, less than 50 ppm, less than 40 ppm, less than 30 ppm, less than 20 ppm, or less than 10 ppm 4-hydroxy-2-butanone 1,3-propanediol, e.g., as determined by GC-MS. In some embodiments, the level of 1,3-propanediol is not detectable in the bioderived 1,3-BG, e.g., by GC-MS.

In some embodiments, the bioderived 1,3-BG has detectable levels of 2,3-butanediol. In some embodiments, the bioderived 1,3-BG has less than 100 ppm, less than 90 ppm, less than 80 ppm, less than 70 ppm, less than 60 ppm, less than 50 ppm, less than 40 ppm, less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, or less than 1 ppm 2,3-butanediol, e.g., as determined by GC-MS. In some embodiments, the level of 2,3-butanediol is not detectable in the bioderived 1,3-BG, e.g., by GC-MS.

In another aspect, provided herein is a process for purifying bioderived 1,3-BG.

In some embodiments, the process for purifying bioderived 1,3-BG can include the steps of culturing a non naturally occurring microbial organism to produce bioderived 1,3-BG in a fermentation broth, and subjecting the fermentation broth to one or more of the following procedures: microfiltration, ultrafiltration, nanofiltration, primary ion exchange, evaporation, polishing ion exchange, column distillation, hydrogenation, active-carbon filtration or adsorbtion, base addition, sodium borohydride (NaBH₄) treatment, and wiped-film evaporation.

In some embodiments, the process for purifying bioderived 1,3-BG comprises (i) microfiltration, followed by (ii) nanofiltration, followed by (iii) primary ion exchange, followed by (iv) evaporation, followed by (v) polishing ion exchange, followed by (vi) distillation. In some embodiments, base addition occurs as a step after ion exchange and before or during a distillation step. In some embodiments, distillation comprises activated carbon treatment. In some embodiments, the activated carbon treatment occur during the distillation process. In some embodiments, the carbon treatment occurs at the end of the distillation process. In some embodiments, distillation is followed by (v) sodium borohydride treatment.

In some embodiments, the process for purifying bioderived 1,3-BG can include distillation of a crude bioderived 1,3-BG mixture or partially purified bioderived 1,3-BG. The distillation can be carried out with a distillation system provided herein to produce a purified bioderived 1,3-BG product. The purified bioderived 1,3-BG product can be or include greater than 90%, 92%, 94%, 96%, 97%, 98%, 99%, 99.5%, 99.7% or 99.9% bioderived 1,3-BG (1,3-BDO) on a weight/weight basis. The distillation system can include or be composed of one or more distillation columns that can be used to remove materials that have a higher or lower boiling point than 1,3-BG by generating streams of materials with boiling points higher or lower than 1,3-BG. The distillation columns can include or contain, for example, random-packing, structured-packing, plates, random- and structured-packing, random-packing and plates, or structured-packing and plates. As is known in the art, many types and configurations of distillation columns are available. The recovery of bioderived 1,3-BG in the purified bioderived 1,3-BG (1,3-BDO) product can be calculated as a percentage of the amount of bioderived 1,3-BG (1,3-BDO) in the purified bioderived 1,3-BG product divided by the amount of bioderived 1,3-BG or target compound in the crude bioderived 1,3-BG mixture that was purified.

A consideration in distillation is to reduce or minimize the amount of heating that a bioderived 1,3-BG or target compound product must undergo through the distillation process. Impurities or even the bioderived 1,3-BG can undergo thermal or chemical decomposition while being heated during distillation. Operating the distillation columns under reduced pressure (less than atmospheric pressure) or vacuum lowers the boiling temperature of the mixture in the distillation column and allows for operating the distillation column at lower temperatures. Any of the columns described in the various embodiments provided herein can be operated under reduced pressure. A common vacuum system can be used with some or all distillation columns to achieve a reduced pressure, or each column can have its own vacuum system. All combinations and permutations of the above exemplary vacuum configurations are included within the present compositions, systems, and methods as provided and described herein. The pressure of a distillation column can be measured at the top or condenser, the bottom or base, or anywhere in between. The pressure at the top of a distillation column can be different than the pressure in the base of the distillation column, and this pressure difference denotes the pressure drop across the distillation column. Different distillation columns of the same embodiment can be operated at different pressures. Pressures in a column can be ambient, less than ambient, or less than 500 mmHg, 200 mmHg, 100 mmHg, 50 mmHg, 40 mmHg, 30 mmHg, 20 mmHg, 15 mmHg, 10 mmHg, or 5 mmHg, for example.

It should be understood that a step of removing higher or lower boiling materials with a distillation column by distillation is not expected to be 100% effective, and that residual amounts of higher or lower boiling materials can still be present in the product stream after a distillation procedure. When it is described that a material is removed by a distillation procedure, it is to be understood that the removal can mean greater than 50%, 60%, 70%, 80%, 90%, 95%, 96%, 97%, 98%, 99%, 99.5% or 99.9% of the material is removed, by distillation, from the feed to a distillation column.

The mixture to be purified can be fed to a distillation column and, depending on the operating conditions, the higher boiling or lower boiling materials can be removed from the mixture. For example, if lower boiling materials are removed, the lower boiling materials are boiled up and removed from the top of the distillation column, and the product-containing stream with the lower boiling materials removed exits from the bottom of the distillation column. This bottom stream can be fed to a next distillation column where the high boiling materials are removed from the product-containing stream. In the next distillation column, the product-containing stream boils up and exits the distillation column from the top, and the higher boiling materials are removed from the bottom of the distillation column, thus providing a more pure product-containing stream. In another example, both the higher boiling and lower boiling materials can be removed from the product-containing stream, where in that case the lower boiling materials are boiled up and removed through the top of the column, the higher boiling materials are removed from the bottom of the column, and a product exits through a side-draw, which allows material to leave the column at an intermediate position between the top and bottom of the distillation column.

In the systems and processes provided herein that include distillation columns, the distillation columns have a number of stages. In some embodiments, the systems or processes of this disclosure have a distillation column with 3 to 80 stages. For example, the distillation column can have 3 to 25 stages, 25 to 50 stages, or 50 to 80 stages. In some embodiments, the distillation column has 8 to 28 stages, e.g., 18 to 14 stages. In some embodiments, the distillation column has 4 stages, 8 stages, 10 stages, 11 stages, 17 stages, 22 stages, 18 stages, 23 stages, 30 stages or 67 stages.

In some embodiments, the process includes (a) subjecting a first bioderived 1,3-BG-containing product stream to a first column distillation procedure to remove materials with a boiling point higher than bioderived 1,3-BG, as a first high boilers stream, to produce a second 1,3-BG-containing product stream; (b) subjecting the second bioderived 1,3-BG-containing product stream to a second column distillation procedure to remove materials with a boiling point lower than bioderived 1,3-BG, to produce a third bioderived 1,3-BG-containing product stream; and (c) subjecting the third bioderived 1,3-BG-containing product stream to a third column distillation procedure to remove materials with boiling points higher than bioderived 1,3-BG as a second high-boilers stream, to produce a fourth bioderived 1,3-BG-containing product stream comprising a purified bioderived 1,3-BG product. In some embodiments, the purified bioderived 1,3-BG product is a bioderived 1,3-BG provided herein.

In some embodiments, the process includes subjecting crude bioderived 1,3-BG mixture or partially purified bioderived 1,3-BG to polishing to produce the first bioderived 1,3-BG-containing product stream of (a). In some embodiments, polishing involves, e.g., ion exchange chromatography, or contacting with activated carbon.

Polishing is a procedure to reduce or remove any remaining salts and/or other impurities in a crude bioderived 1,3-BG mixture, or partially purified bioderived 1,3-BG. The polishing can include contacting the crude bioderived 1,3-BG (1,3-BDO), or partially purified bioderived 1,3-BG, with one or a number of materials that can react with or adsorb the impurities in the crude bioderived 1,3-BG mixture or or partially purified bioderived 1,3-BG. The materials used in the polishing can include ion exchange resins, activated carbon, or adsorbent resins, such as, for example, DOWEX™ 22, DOWEX™ 88, OPTIPORE™ L493, AMBERLITE™ XAD761 or AMBERLITE™ FPX66, or mixtures of these resins, such as a mixture of DOWEX™ 22 and DOWEX™ 88.

In some embodiments, the polishing is or includes a polishing ion exchange. The polishing ion exchange can be used to remove any residual salts, color bodies and color precursors before further purification. The polishing ion exchange can include an anion exchange, a cation exchange, both a cation exchange and anion exchange, or can be or include a mixed cation-anion exchange, which includes both cation exchange and anion exchange resins. In certain embodiments, the polishing ion exchange is or includes an anion exchange followed by a cation exchange, a cation exchange followed by an anion exchange, or a mixed cation-anion exchange. In certain embodiment, the polishing ion exchange is or includes an anion exchange. The polishing ion exchange is or includes both strong cation and strong anion exchange, or is or includes strong anion exchange without other polishing cation exchange or polishing anion exchange. In some embodiments, the polishing ion exchange occurs after a water removal step such as evaporation, and prior to a subsequent distillation.

In some embodiments, the process includes subjecting a crude bioderived 1,3-BG mixture or partially purified bioderived 1,3-BG to a dewatering column distillation procedure to remove materials with a boiling point lower than bioderived 1,3-BG from the crude bioderived 1,3-BG mixture or partially purified bioderived 1,3-BG to produce the first bioderived 1,3-BG-containing product stream of (a).

In some embodiments, the process includes subjecting a crude bioderived 1,3-BG mixture or partially purified bioderived 1,3-BG to polishing and subjecting the resulting crude bioderived 1,3-BG mixture or partially purified bioderived 1,3-BG to a dewatering column distillation procedure to reduce or remove materials with a boiling point lower than bioderived 1,3-BG from the resulting crude bioderived 1,3-BG mixture to produce the first bioderived 1,3-BG-containing product stream of (a). In some embodiments, polishing involves, e.g., ion exchange chromatography, or contacting with activated carbon.

The reflux rate in a distillation system or process is the ratio between the boil up rate and the take-off rate. In other words, the reflux rate is the ratio between the amount of reflux that goes back down the distillation column and the amount of reflux that is collected in the receiver (distillate). For example, a reflux rate of 2:1 indicates that twice as much reflux (e.g., in volume or by weight) goes back down the distillation column as is collected in the distillate.

In some embodiments, the reflux ratio in the dewatering column, or the first, second or third distillation column in a process or system provided herein is 1:1 or more, 2:1 or more, 3:1 or more, 4:1 or more, 5:1 or more, 6:1 or more, 7:1 or more, 8:1 or more, 9:1 or more, or 10:1 or more.

In some embodiments, the reflux ratio in the dewatering column, or the first, second or third distillation column in a process or system provided herein is 1:1 or less, 1:2 or less, 1:3 or less, 1:4 or less, 1:5 or less, 1:6 or less, 1:7 or less, 1:8 or less, 1:9 or less, or 1:10 or less.

In some embodiments, the process includes adding a base to a bioderived 1,3-BG-containing product stream before or after any one of (a), (b), or (c). In some embodiments, the base is added to the bioderived 1,3-BG containing product stream before (a). In some embodiments, the base is added to the bioderived 1,3-BG containing product stream before crude bioderived 1,3-BG or partially purified bioderived 1,3-BG is subjected to polishing. In some embodiments, polishing involves or includes, e.g., ion exchange chromatography, or contacting with activated carbon, or both. In some embodiments, the base is added to the bioderived 1,3-BG containing product stream after crude bioderived 1,3-BG or partially purified bioderived 1,3-BG is subjected to polishing. In some embodiments, the base is added before the crude bioderived 1,3-BG mixture or partially purified bioderived 1,3-BG resulting from polishing is subjected to a dewatering column. In some embodiments, the base is added after the crude bioderived 1,3-BG mixture or partially purified bioderived 1,3-BG resulting from polishing is subjected to a dewatering column. In some embodiments, the base is added to the bioderived 1,3-BG containing product stream after (a). In some embodiments, the base is added to the bioderived 1,3-BG containing product stream between (a) and (b). In some embodiments, the base is added to the bioderived 1,3-BG containing product stream before (b). In some embodiments, the base is added to the bioderived 1,3-BG containing product stream after (b). In some embodiments, the base is added to the bioderived 1,3-BG containing product stream between (b) and (c). In some embodiments, the base is added to the bioderived 1,3-BG containing product stream before (c). In some embodiments, the base is added to the bioderived 1,3-BG containing product stream after (c).

In some embodiments, the base is added to a reboiler of the dewatering column, or the first, second, or third distillation column, or to a combination thereof.

In some embodiments, the base is added in an alkali reactor, such as a circulating tube-type reactor.

In some embodiments, the base can include, e.g., an alkali metal compound, such as sodium hydroxide, potassium hydroxide, sodium (bi)carbonate, ammonium hydroxide, or a combination thereof.

In some embodiments, the base is added in an amount of 0.05% to 10% by weight based on the crude bioderived 1,3-BG mixture or partially purified bioderived 1,3-BG, e.g., 0.05% to 1%, 1% to 2%, 2% to 3%, 3%-4%, 4%-5%, 5%-6%, 6%-7%, 7%-8%, 8%-9%, or 9%-10% by weight.

In some embodiments, base addition occurs at a temperature of 90-140° C. in the alkali reactor, e.g., 90-110° C., 110-130° C., or 120-140° C.

In some embodiments, the retention time in the alkali reactor is 5 to 120 minutes, e.g., 5 to 15 minutes, 10 to 30 minutes, 20 to 40 minutes, 30 to 50 minutes, 40 to 60 minutes, 50 to 70 minutes, 60 to 80 minutes, 70 to 90 minutes, 80 to 100 minutes, 90 to 110 minutes, or 100 to 120 minutes.

In some embodiments, base addition is followed by dealkalization, e.g., using a thin film evaporator. In some embodiments, during dealkalization, the base added to the crude bioderived 1,3-BG mixture or partially purified bioderived 1,3-BG is removed from a bioderived 1,3-BG containing product stream along with high-boiling materials.

In some embodiments, the process includes treating a bioderived 1,3-BG containing product stream with a hydrogenation reaction before or after any one of (a), (b), or (c). In some embodiments, the process includes treating a bioderived 1,3-BG containing product stream with a hydrogenation reaction before (a). In some embodiments, the process includes treating a bioderived 1,3-BG containing product stream with a hydrogenation reaction between (a) and (b). In some embodiments, the process includes treating a bioderived 1,3-BG containing product stream with a hydrogenation reaction after (a). In some embodiments, the process includes treating a bioderived 1,3-BG containing product stream with a hydrogenation reaction before (b). In some embodiments, the process includes treating a bioderived 1,3-BG containing product stream with a hydrogenation reaction after (b). In some embodiments, the process includes treating a bioderived 1,3-BG containing product stream with a hydrogenation reaction between (b) and (c). In some embodiments, the process includes treating a bioderived 1,3-BG containing product stream with a hydrogenation reaction before (c). In some embodiments, the process includes treating a bioderived 1,3-BG containing product stream with a hydrogenation reaction after (c).

In some embodiments, the hydrogenation reaction reduces the concentration of 3-hydroxy-butanal or 4-hydroxy-2-butanone in the second bioderived 1,3-BG containing product stream by 50% or more, 60% or more, 70% or more, 80% or more, 90% or more, or 95% or more.

In some embodiments, the hydrogenation reaction reduces the UV absorption at 270 nm or at 220 nm in the second bioderived 1,3-BG containing product stream by 50% or more, 60% or more, 70% or more, 80% or more, 90% or more, or 95% or more.

In some embodiments, the process includes contacting a bioderived 1,3-BG containing product stream with activated carbon. In some embodiments, the activated carbon is chemically activated carbon. “Chemically activated carbon” as used herein, refers to activated carbon which has been activated by treatment with a chemical as opposed to oxidized with air or other gasses. In some embodiments, chemically activated carbon is given a second activation with steam to impart physical properties not developed by chemical activation. Chemical activating agents that can be used include phosphoric acid; sulfuric acid; zinc chloride; potassium sulfide; potassium thiocyanate; alkali metal hydroxides, carbonates; sulfides and sulfates; as well as alkaline earth carbonates; chlorides; sulfates; and phosphates. In some embodiments, the chemically activated carbon used in the processes and systems provided herein is a wood-based (sawdust) activated carbon, activated with phosphoric acid. Exemplary chemically activated carbon is commercially available, e.g., MeadWestvaco Corp. (Richmond, VA) Nuchar® WV-B grade activated carbon materials.

The activated carbon can, e.g., be in a pulverized or granular form. In some embodiments, the activated carbon is coal, wood, or coconut shell based. In some embodiments, the activated carbon is steam activated. In some embodiments, the activated coal is acid washed. In some embodiments, the activated carbon can include Cabot Darco S-51A M-1967 (Darco; Cabot Corp., Boston, MA), Calgon FILTRASORB 300 (FS 300; Calgon Carbon Corp., Moon Township, PA), or Calgon CPG-LF (CPG-LF; Calgon Carbon Corp., Moon Township, PA).

In some embodiments, bioderived 1,3-BG treated with activated carbon is “consumed” without further purification when furnished to a customer and/or incorporated into another composition immediately after activated carbon treatment; e.g., without further subsequent purification steps such as distillation and the like.

In some embodiments, the process includes contacting the first bioderived 1,3-BG containing product stream with activated carbon. In some embodiments, the process includes contacting the second bioderived 1,3-BG containing product stream with activated carbon. In some embodiments, the process includes contacting the third bioderived 1,3-BG containing product stream with activated carbon. In some embodiments, the process includes contacting the second high-boilers stream with activated carbon.

In some embodiments, contacting a bioderived 1,3-BG containing product stream with activated carbon reduces the concentration of 3-hydroxy-butanal or 4-hydroxy-2-butanone in the second bioderived 1,3-BG containing product stream by 50% or more, 60% or more, 70% or more, 80% or more, 90% or more, or 95% or more.

In some embodiments, the process includes contacting a bioderived 1,3-BG containing product stream with sodium borohydride (NaBH₄). In some embodiments, the process includes contacting the first bioderived 1,3-BG containing product stream with NaBH₄. In some embodiments, the process includes contacting the second bioderived 1,3-BG containing product stream with NaBH₄. In some embodiments, the process includes contacting the third bioderived 1,3-BG containing product stream with NaBH₄. In some embodiments, the process includes contacting the second high-boilers stream with NaBH₄.

In some embodiments, contacting a bioderived 1,3-BG containing product stream with NaBH₄ reduces the concentration of 3-hydroxy-butanal or 4-hydroxy-2-butanone in the distillate of the second bioderived 1,3-BG containing product stream by 50% or more, 60% or more, 70% or more, 80% or more, 90% or more, or 95% or more. In some embodiments, contacting a bioderived 1,3-BG containing product stream with NaBH₄ reduces the UV absorption at 270 nm or at 220 nm in the second bioderived 1,3-BG containing product stream by 50% or more, 60% or more, 70% or more, 80% or more, 90% or more, or 95% or more.

In some embodiments, the process includes subjecting the first high-boilers stream to wiped-film evaporation (WFE) to produce a WFE distillate and subjecting the WFE distillate to a first column distillation procedure.

In some embodiments, the process includes subjecting the second high-boilers stream to WFE to produce a WFE distillate and subjecting the WFE distillate to a third column distillation procedure.

In some embodiments, the distillation includes subjecting a first high-boilers stream from the distillation to WFE to produce a WFE distillate. The WFE distillate can be further subjected to a first column distillation procedure in a system provided herein. In some embodiments, the WFE distillate can be further subjected to a fourth column distillation procedure in a system provided herein.

A WFE, also known as thin film evaporation, can be useful for relatively quickly separating volatile from less volatile components where components include those that are heat sensitive, viscous and tend to foul heated surfaces (e.g., amino acids, sugars and other components often found in fermentation broths). Typically in embodiments of the systems and processes described herein, the vaporizable component (distillate) from a wiped-film evaporator (“WFE”) contains bioderived 1,3-BG. Thus, as utilized in the systems and processes described herein, the WFE is a distillation component that increases product yields by recovery of bioderived 1,3-BG from the heavies material that would otherwise be disposed. For example, in a column distillation system or process where a crude bioderived 1,3-BG mixture (or partially purified bioderived 1,3-BG, such as a bioderived 1,3-BG (1,3-BDO) product stream from a dewatering column) is fed into a given distillation column from which 1,3-BG is removed as a distillate (“low-boilers”) and the bottoms purge (“high-boilers”) from the distillation column (which would otherwise be disposed of) is subjected to wiped-film evaporation; the WFE's 1,3-BG-containing distillate is put back into the column distillation system or process to increase the recovery of 1,3-BG. Heat times in a wiped-film evaporator can be short to minimize decomposition.

In some embodiments, the WFE is a short path distillator (SPD). In some embodiments, the WFE is a vertical WFE. In some embodiments, the WFE is a horizontal WFE.

Wiped-film evaporators can be operated under vacuum conditions, such as less than 50 mmHg, 25 mmHg, 10 mmHg, 1 mmHg, 0.1 mmHg, 0.01 mmHg or even lower. Operating conditions for wiped-film evaporation can, for example, be with a pressure ranging from about 0.1 mmHg to 25 mmHg, about 1 mmHg to 10 mmHg, about 2 mmHg to 7.5 mmHg, about 4 mmHg to 7.5 mmHg, or about 4 mmHg to 15 mmHg, and a temperature range from about 100° C. to 150° C., 110° C. to 150° C., 115° C. to 150° C., 115° C. to 140° C., 115° C. to 130° C. or 125° C. to 150° C.

In some embodiments, the WFE can be operated at a temperature below 160° C. In some embodiments, the WFE can be operated at a temperature between 145° C. and 155° C. In some embodiments, the WFE can be operated under vacuum. In some embodiments, operating conditions for wiped-film evaporators include a temperature from about 145° C. to 155° C. and a vacuum from about 4 mmHg to 15 mmHg.

In some embodiments, the processes for purifying bioderived 1,3-BG provided herein include one or more of fermentation, cell separation, salt separation, evaporation, or a combination thereof. In some embodiments, the process includes fermentation, followed by cell separation, followed by salt separation, followed by evaporation. In some embodiments, fermentation, cell separation, salt separation, and evaporation yield a crude bioderived 1,3-BG mixture or partially purified bioderived 1,3-BG that can be fed into a polishing column (e.g., polishing ion-exchange), a dewatering column, or a first distillation column in a process or system provided herein.

In some embodiments, the process includes fermentation. In some embodiments, fermentation includes culturing a non-naturally occurring microbial organism to produce bioderived 1,3-BG in a fermentation broth. Exemplary non-naturally occurring microbial organisms and methods for producing bioderived 1,3-BG in a fermentation broth are described, e.g., in WO 2010/127319 A2 and WO 2011/071682 A1, the entire contents of each of which are incorporated by reference herein.

In some embodiments, the process includes cell separation. In some embodiments, cell separation includes separating a liquid fraction from a fermentation broth enriched in bioderived 1,3-BG from a sold fraction comprising cells. In some embodiments, the separating includes centrifugation or filtration, or a combination thereof. In some embodiments, the filtration includes microfiltration, ultrafiltration, or nanofiltration, or a combination thereof. In some embodiments, the filtration consists of microfiltration. In some embodiments, the filtration consists of ultrafiltration. In some embodiments, the filtration consists of microfiltration and nanofiltration. In some embodiments, the filtration consists of ultrafiltration and nanofiltration.

Centrifugation can be used to provide a crude bioderived 1,3-BG mixture or partially purified bioderived 1,3-BG substantially free of solids, including cell mass. Depending on the centrifuge configuration and size, operating speeds can vary from less than 500 rpm, generally from 500 rpm to 12,000 rpm or more than 12,000 rpm. The rpm from 500 to 12,000 can produce a centrifugal force of up to and over 15,000 times the force of gravity. Many centrifuge configurations for removal of cells and solids from a fermentation broth are known in the art and can be employed in the systems and processes provided herein. Such configurations include, for example, a disc-stack centrifuge and a decanter, or solid bowl centrifuge. Centrifugation can occur batch-wise or in a continuous fashion. All combinations of centrifugation configurations well known in the art can be employed in the systems and processes provided herein.

Microfiltration, for example, involves a low-pressure membrane process for separating colloidal and suspended particles in the range of about 0.05-10 microns. Useful configurations include cross-flow filtration using spiral-wound, hollow fiber, or flat sheet (cartridge) microfiltration elements. Microfiltration includes filtering through a membrane having pore sizes from about 0.05 microns to about 10.0 microns. Microfiltration membranes can have nominal molecular weight cut-offs (MWCO) of about 20,000 Daltons and higher. The term molecular weight cut-off is used to denote the size of particle, including polypeptides, or aggregates of peptides, that will be approximately 90% retained by the membrane. Polymeric, ceramic, or steel microfiltration membranes can be used to separate cells. Ceramic or steel microfiltration membranes have long operating lifetimes including up to or over 10 years. Microfiltration can be used in the clarification of fermentation broth. For example, microfiltration membranes can have pore sizes from about 0.05 microns to 10 micron, or from about 0.05 microns to 2 microns, about 0.05 microns to 1.0 micron, about 0.05 microns to 0.5 microns, about 0.05 microns to 0.2 microns, about 1.0 micron to 10 microns, or about 1.0 micron to 5.0 microns, or membranes can have a pore size of about 0.05 microns, about 0.1 microns, or about 0.2 microns. For example, microfiltration membranes can have a MWCO from about 20,000 Daltons to 500,000 Daltons, about 20,000 Daltons to 200,000 Daltons, about 20,000 Daltons to 100,000 Daltons, about 20,000 Daltons to 50,000 Daltons, or with about 50,000 Daltons to 300,000 Daltons; or with a MWCO of about 20,000 Daltons, about 50,000 Dalton, about 100,000 Daltons or about 300,000 Daltons can be used in separating cell and solids from the fermentation broth.

Ultrafiltration is a selective separation process through a membrane using pressures up to about 145 psi (10 bar). Useful configurations include cross-flow filtration using spiral-wound, hollow fiber, or flat sheet (cartridge) ultrafiltration elements. These elements consist of polymeric or ceramic membranes with a molecular weight cut-off of less than about 200,000 Daltons. Ceramic ultrafiltration membranes are also useful since they have long operating lifetimes of up to or over 10 years. Ceramics have the disadvantage of being much more expensive than polymeric membranes. Ultrafiltration concentrates suspended solids and solutes of molecular weight greater than about 1,000 Daltons. Ultrafiltration includes filtering through a membrane having nominal molecular weight cut-offs (MWCO) from about 1,000 Daltons to about 200,000 Daltons (pore sizes of about 0.005 to 0.1 microns). For example, ultrafiltration membranes can have pore sizes from about 0.005 microns to 0.1 micron, or from about 0.005 microns to 0.05 microns, about 0.005 microns to 0.02 micron, or about 0.005 microns to 0.01 microns. For example, ultrafiltration membranes can have a MWCO from about 1,000 Daltons to 200,000 Daltons, about 1,000 Daltons to 50,000 Daltons, about 1,000 Daltons to 20,000 Daltons, about 1,000 Daltons to 5,000 Daltons, or with about 5,000 Daltons to 50,000 Daltons. Using ultrafiltration the permeate liquid will contain low-molecular-weight organic solutes, such as bioderived 1,3-BG, media salts, and water. The captured solids can include, for example, residual cell debris, DNA, and proteins. Diafiltration techniques well known in the art can be used to increase the recovery of bioderived 1,3-BG in the ultrafiltration step.

A further filtration procedure called nanofiltration can be used to separate out certain materials by size and charge, including carbohydrates, inorganic and organic salts, residual proteins and other high molecular weight impurities that remain after the previous filtration step. This procedure can allow the recovery of certain salts without prior evaporation of water, for example. Nanofiltration can separate salts, remove color, and provide desalination. In nanofiltration, the permeate liquid generally contains monovalent ions and low-molecular-weight organic compounds as exemplified by bioderived 1,3-BG. Nanofiltration includes filtering through a membrane having nominal molecular weight cut-offs (MWCO) from about 100 Daltons to about 2,000 Daltons (pore sizes of about 0.0005 to 0.005 microns). For example, nanofiltration membranes can have a MWCO from about 100 Daltons to 500 Daltons, about 100 Daltons to 300 Daltons, or about 150 Daltons to 250 Daltons. The mass transfer mechanism in nanofiltration is diffusion. The nanofiltration membrane allows the partial diffusion of certain ionic solutes (such as sodium and chloride), predominantly monovalent ions, as well as water. Larger ionic species, including divalent and multivalent ions, and more complex molecules are substantially retained (rejected). Larger non-ionic species, such as carbohydrates are also substantially retained (rejected). Nanofiltration is generally operated at pressures from 70 psi to 700, psi, from 200 psi to 650 psi, from 200 psi to 600 psi, from 200 psi to 450 psi, from 70 psi to 400 psi, of about 400 psi, of about 450 psi or of about 500 psi.

One embodiment of a nanofiltration has a membrane with a molecular weight cut off of about 200 Daltons that rejects, for example, about 99% of divalent salts such as magnesium sulfate. A certain embodiment would have a nanofiltration membrane with a molecular weight cut off of about 150-300 Daltons for uncharged organic molecules.

In some embodiments, the process includes salt separation. In some embodiments, salt separation occurs prior to water removal. In some embodiments, salt removal includes nanofiltration. In some embodiments, salt removal includes ion-exchange. In some embodiments, salt removal includes nanofiltration and ion exchange.

Ion exchange can be used to remove salts from a mixture, such as for example, a fermentation broth. Ion exchange elements can take the form of resin beads as well as membranes. Frequently, the resins can be cast in the form of porous beads. The resins can be or include cross-linked polymers having active groups in the form of electrically charged sites. At these sites, ions of opposite charge are attracted, but can be replaced by other ions depending on their relative concentrations and affinities for the sites. Ion exchange resins can be cationic or anionic, for example. Factors that determine the efficiency of a given ion exchange resin include the favorability for a given ion, and the number of active sites available. To maximize the active sites, large surface areas can be useful. Thus, small porous particles are useful because of their large surface area per unit volume.

The anion exchange resins can be strongly basic or weakly basic anion exchange resins, and the cation exchange resin can be strongly acidic or weakly acidic cation exchange resin. Non-limiting examples of ion-exchange resin that are strongly acidic cation exchange resins include AMBERJET™ 1000 Na, AMBERLITE™ IR 10 or DOWEX™ 88; weakly acidic cation exchange resins include AMBERLITE™ IRC86 or DOWEX™ MAC3; strongly basic anion exchange resins include AMBERJET™ 4200 Cl or DOWEX™ 22; and weakly basic anion exchange resins include AMBERLITE™ IRA96, DOWEX™ 77 or DOWEX™ Marathon WMA. Ion exchange resins can be obtained from a variety of manufacturers such as Dow, Purolite, Rohm and Haas, Mitsubishi or others.

In some embodiments, primary ion exchange chromatography is performed using DOWEX™ 88 (cation exchange) and DOWEX™ 77 (anion exchange) resins.

In some embodiments, polishing ion exchange chromatography is performed using DOWEX™ 88 (cation exchange) and DOWEX™ 22 (anion exchange) resins.

A primary ion exchange can be utilized for the removal of salts. The primary ion exchange can include, for example, both a cation exchange or an anion exchange, or a mixed cation-anion exchange, which include both cation exchange and anion exchange resins. In certain embodiments, primary ion exchange can be cation exchange and anion exchange in any order. In some embodiments, the primary ion exchange is an anion exchange followed by a cation exchange, or a cation exchange followed by an anion exchange, or a mixed cation-anion exchange. In certain embodiments, the primary ion exchange is an anion exchange, or a cation exchange. More than one ion exchange of a given type, can be used in the primary ion exchange. For example, the primary ion exchange can include a cation exchange, followed by an anion exchange, followed by a cation exchange and finally followed by an anion exchange.

In certain embodiments, the primary ion exchange uses a strongly acidic cation exchange and a weakly basic anion exchange Ion exchange, for example, primary ion exchange, can be carried out at temperatures from 20° C. to 60° C., from 30° C. to 60° C., 30° C. to 50° C., 30° C. to 40° C. or 40° C. to 50° C.; or at about 30° C., about 40° C., about 50° C., or about 60° C. Flow rates in ion exchange, such as primary ion exchange, can be from 1 bed volume per hour (BV/h) to 10 BV/h, 2 BV/h to 8 BV/h, 2 BV/h to 6 BV/h, 2 BV/h to 4 BV/h, 4 BV/h to 6 BV/h, 4 BV/h to 8 BV/h, 4 BV/h to 10 BV/h or 6 BV/h to 10 BV/h.

In some embodiments, the bioderived 1,3-BG product obtained after salt removal and/or water removal is a crude bioderived 1,3-BG mixture or partially purified bioderived 1,3-BG. The crude bioderived 1,3-BG or partially purified bioderived 1,3-BG or target compound mixture obtained is at least 50%, 60%, 70%, 80%, 85% or 90% 1,3-BG, and is less than 50%, 40%, 30%, 20%, 15%, 10% or 5% water, e.g., on a weight/weight basis.

In some embodiments, the process includes evaporation to remove water from a bioderived 1,3-BG product. There are many types and configurations of evaporators well known to those skilled in the art that are available for water removal. An evaporator is a heat exchanger in which a liquid is boiled to give a vapor that is also a low pressure steam generator. This steam can be used for further heating in another evaporator called another “effect.” Removing water is accomplished by evaporation with an evaporator system which includes one or more effects. In some embodiments, a double- or triple-effect evaporator system can be used to separate water from bioderived 1,3-BG. Any number of multiple-effect evaporator systems can be used in the removal of water. A triple effect evaporator, or other evaporative apparatus configuration, can include dedicated effects that are evaporative crystallizers for salt recovery, for example the final effect of a triple effect configuration. Alternatively, mechanical vapor recompression or thermal vapor recompression evaporators can be utilized to reduce the energy required for evaporating water beyond what can be achieved in standard multiple effect evaporators.

Examples of evaporators include a falling film evaporator (which can be a short path evaporator), a forced circulation evaporator, a plate evaporator, a circulation evaporator, a fluidized bed evaporator, a rising film evaporator, a counterflow-trickle evaporator, a stirrer evaporator and a spiral tube evaporator.

In some embodiments, the purified bioderived 1,3-BG product produced in a process provided herein includes a bioderived 1,3-BG provided herein.

In some embodiments, the purified bioderived 1,3-BG product is collected as a distillate of the third column distillation procedure.

In another aspect, provided herein is bioderived 1,3-BG produced by a process provided herein.

In some embodiments, the process includes subjecting a crude bioderived 1,3-BG mixture or partially purified bioderived 1,3-BG to a dewatering column distillation procedure to remove materials with a boiling point lower than bioderived 1,3-BG from the crude bioderived 1,3-BG mixture or partially purified bioderived 1,3-BG to produce a first bioderived 1,3-BG-containing product stream; subjecting the first bioderived 1,3-BG-containing product stream to a first column distillation procedure to remove materials with a boiling point higher than bioderived 1,3-BG, as a first high boilers stream, to produce a second 1,3-BG-containing product stream; optionally adding a base to the second 1,3-BG-containing product stream; optionally treating the second 1,3-BG-containing product stream with a hydrogenation reaction; subjecting the second bioderived 1,3-BG-containing product stream to a second column distillation procedure to remove materials with a boiling point lower than bioderived 1,3-BG, to produce a third bioderived 1,3-BG-containing product stream; subjecting the third bioderived 1,3-BG-containing product stream to a third column distillation procedure to remove materials with boiling points higher than bioderived 1,3-BG as a second high-boilers stream, to produce a fourth 1,3-BG-containing product stream, optionally subjecting the fourth 1,3-BG-containing product stream to activated carbon, to produce a purified bioderived 1,3-BG product, wherein the purified bioderived 1,3-BG product is a bioderived 1,3-BG provided herein.

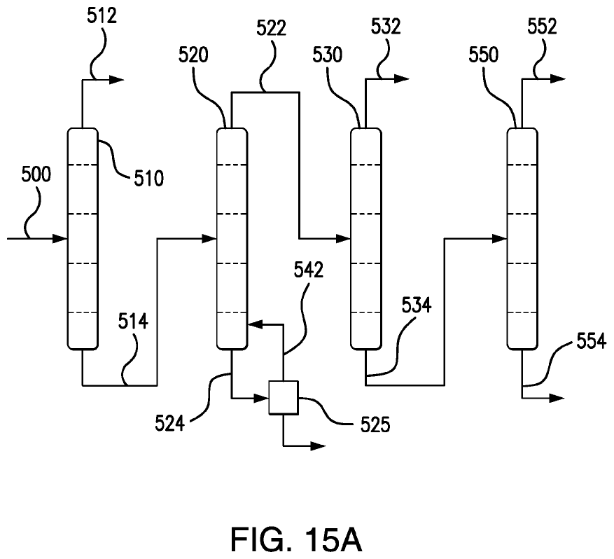

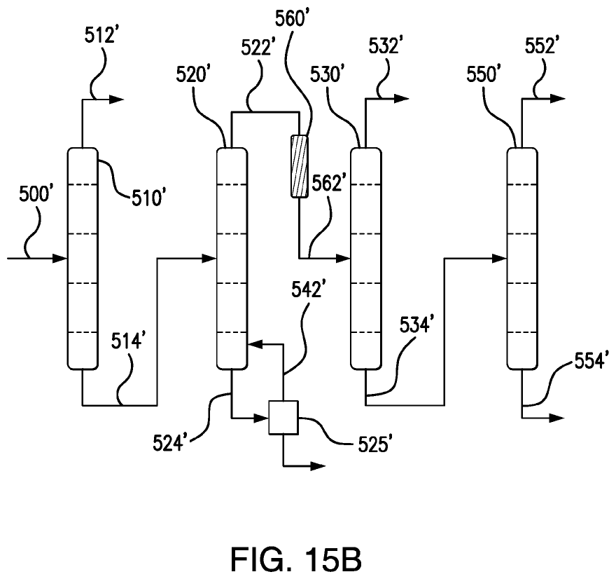

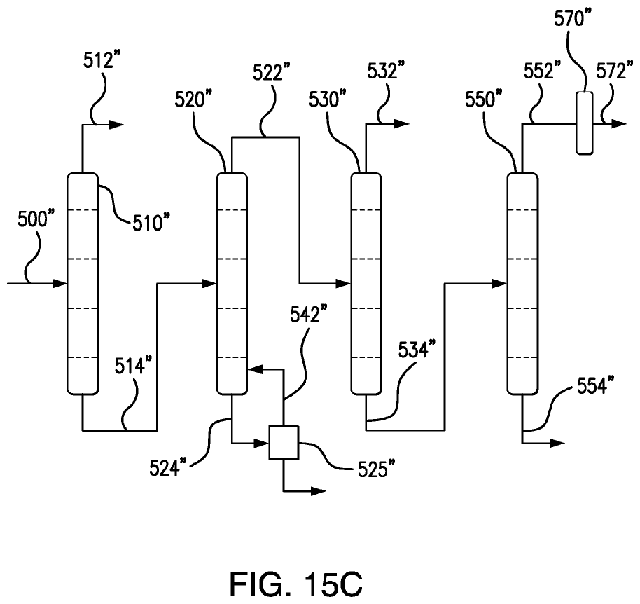

In another aspect, provided herein is a system for purifying bioderived 1,3-BG, comprising a first distillation column receiving a first bioderived 1,3-BG containing product stream generating a first stream of materials with boiling points higher than 1,3-BG, and a second bioderived 1,3-BG-containing product stream; a second distillation column receiving the second bioderived 1,3-BG-containing product stream generating a stream of materials with boiling points lower than 1,3-BG, and a third bioderived 1,3-BG-containing product stream; and a third distillation column receiving the third 1,3-BG-containing product stream at a feed point and generating a second stream of materials with boiling points higher than 1,3-BG, and a fourth bioderived 1,3-BG-containing product stream comprising a purified bioderived 1,3-BG product. In some embodiments, the fourth bioderived 1,3-BG-containing product stream consists essentially of a bioderived 1,3-BG provided herein. See, e.g., FIG. 15A.

In some embodiments, the system includes a polishing column receiving a crude bioderived 1,3-BG mixture or partially purified bioderived 1,3-BG generating a crude bioderived 1,3-BG mixture or partially purified bioderived 1,3-BG of reduced salt content. In some embodiments, the crude bioderived 1,3-BG mixture or partially purified bioderived 1,3-BG of reduced salt content is the first bioderived 1,3-BG-containing product stream received by the first distillation column. In some embodiments, the crude bioderived 1,3-BG mixture or partially purified bioderived 1,3-BG of reduced salt content is received by a dewatering column. In some embodiments, the polishing column is an ion exchange chromatography column, or includes activated carbon.

In some embodiments, the system includes a dewatering column receiving a crude bioderived 1,3-BG mixture or partially purified bioderived 1,3-BG generating a stream of materials with boiling points lower than 1,3-BG and the first bioderived 1,3-BG-containing product stream. In some embodiments, the crude bioderived 1,3-BG mixture or partially purified bioderived 1,3-BG is of reduced salt content and generated by a polishing column. In some embodiments, the polishing column is an ion exchange chromatography column, or includes activated carbon.

In some embodiments, the system includes a polishing column receiving a crude bioderived 1,3-BG mixture or partially purified bioderived 1,3-BG generating a crude bioderived 1,3-BG mixture or partially purified bioderived 1,3-BG of reduced salt content and a dewatering column receiving the crude bioderived 1,3-BG mixture or partially purified bioderived 1,3-BG of reduced salt content generating a stream of materials with boiling points lower than 1,3-BG and the first bioderived 1,3-BG containing product stream. In some embodiments, the polishing column is an ion exchange chromatography column, or includes activated carbon.

In some embodiments, the dewatering column in a four-column system has 5 to 15 stages. In some embodiments, the dewatering column in a four-column system has 10 stages.

In some embodiments, the first column in a four-column system has 10 to 40 stages. In some embodiments, the first column in a four-column system has 15 to 35 stages. In some embodiments, the first column in a four-column system has 18 stages. In some embodiments, the first column in a four-column system has 30 stages.

In some embodiments, the second column in a four-column system has 10 to 40 stages. In some embodiments, the second column in a four-column system has 15 to 35 stages. In some embodiments, the second column in a four-column system has 18 stages. In some embodiments, the second intermediate column in a four-column system has 30 stages.

In some embodiments, the third column in a four-column system has 5 to 35 stages. In some embodiments, the third column in a four-column system has 10 to 30 stages. In some embodiments, the third column in a four-column system has 15 to 25 stages. In some embodiments, the third column in a four-column system has 18 stages. In some embodiments, the third column in a four column system has 23 stages.

In some embodiments of a four-column system, the dewatering column has 10 stages, the first column has 30 stages, the second intermediate column has 30 stages and the third column has 23 stages.

In some embodiments of a four-column system, the dewatering column has 8 stages, the first column has 18 stages, the second column has 18 stages and the third column has 18 stages.

In some embodiments, the system includes an alkali reactor. In some embodiments, the alkali reactor can receive a crude bioderived 1,3-BG mixture or partially purified bioderived 1,3-BG and generate a crude bioderived 1,3-BG mixture or partially purified bioderived 1,3-BG having an elevated pH level that can be fed into a polishing column or a dewatering column. In some embodiments, the polishing column is an ion exchange chromatography column, or includes activated carbon. In some embodiments, the alkali reactor can receive a crude bioderived 1,3-BG mixture or partially purified bioderived 1,3-BG of reduced salt content and generate a crude bioderived 1,3-BG mixture or partially purified bioderived 1,3-BG having an elevated pH level that can be fed into a dewatering column. In some embodiments, the alkali reactor can receive a first bioderived 1,3-BG containing product stream and generate a first bioderived 1,3-BG containing product stream having an elevated pH level that can be fed into a first distillation column. In some embodiments, the alkali reactor can receive a second bioderived 1,3-BG containing product stream and generate a second bioderived 1,3-BG containing product stream having an elevated pH level that can be fed into a second distillation column. In some embodiments, the alkali reactor can receive a third bioderived 1,3-BG containing product stream and generate a third bioderived 1,3-BG containing product stream having an elevated pH level that can be fed into a third distillation column.

In some embodiments, the systems provided herein that include an alkali reactor also include a dealkalization tower to remove the base used in the alkali reactor and resulting high-boiling materials from the tower bottom. In some embodiments, the dealkalization tower is a thin-film evaporator. In some embodiments, the evaporator used as a dealkalization tower is a natural flow-down type thin film evaporator or a forced stirring type thin film evaporator having a short retention time to suppress thermal hysteresis to the process fluid. In some embodiments, in the evaporator, evaporation is carried out at a reduced pressure of 100 torr or less, e.g., 90 torr or less, 80 torr or less, 70 torr or less, 60 torr or less, 50 torr or less, 40 torr or less, 30 torr or less, 20 torr or less, 10 torr or less, or 5 torr or less. In some embodiments, the evaporation temperatures range between 90° C. and 120° C.

In some embodiments, the system includes a hydrogenation reactor constructed to treat the a bioderived 1,3-BG containing product stream. In some embodiments, the hydrogenation reactor can receive a crude bioderived 1,3-BG mixture or partially purified bioderived 1,3-BG and generate a hydrogenated crude bioderived 1,3-BG mixture or partially purified bioderived 1,3-BG that can be fed into a polishing column or a dewatering column. In some embodiments, the polishing column is an ion exchange chromatography column, or includes activated carbon. In some embodiments, the hydrogenation reactor can receive a crude bioderived 1,3-BG mixture or partially purified bioderived 1,3-BG of reduced salt content and generate a hydrogenated crude bioderived 1,3-BG mixture or partially purified bioderived 1,3-BG that can be fed into a dewatering column. In some embodiments, the hydrogenation reactor can receive a first bioderived 1,3-BG containing product stream and generate a hydrogenated first bioderived 1,3-BG containing product stream that can be fed into a first distillation column. In some embodiments, the hydrogenation reactor can receive a second bioderived 1,3-BG containing product stream and generate a hydrogenated second bioderived 1,3-BG containing product stream that can be fed into a second distillation column. In some embodiments, the hydrogenation reactor can receive a third bioderived 1,3-BG containing product stream and generate a hydrogenated third bioderived 1,3-BG containing product stream that can be fed into a third distillation column.

A hydrogenation unit can be used to react hydrogen with a material using a catalyst under pressure and heat. Hydrogenation units can be operated, for example, in batch mode or continuously. Some types of catalysts used can be metals on a support. Non-limiting examples of metals useful for hydrogenation include palladium, platinum, nickel, and ruthenium. Non-limiting examples of supports for the metal catalysts include carbon, alumina, and silica. The catalyst can also be, for example, a sponge metal type, such a RANEY-Nickel. Other nickel catalysts are available from commercial vendors, for example, NISAT 310™, E-3276 (BASF, Ludwigshafen, Germany), RANEY® 2486, or E-474 TR (Mallinckrodt Co., Calsicat Division, PA, USA). Pressures can include at least 50 psig, 100 psig, 200 psig, 300 psig, 400 psig, 500 psig, 600 psig or 1000 psig of hydrogen pressure, or from about 100 psig to 1000 psig, from about 200 psig to 600 psig, or from about 400 psig to 600 psig, of hydrogen pressure. Temperatures can be from ambient to 200° C., from about 50° C. to 200° C., from about 80° C. to 150° C., from about 90° C. to 120° C., from about 100° C. to 130° C., or from about 125° C. to 130° C. Hydrogenation preferably occurs after a distillation procedure that includes a substantially removing material with boiling points higher than 1,3-BG, e.g. unfermented sugars, nitrogen-containing compounds, otherwise the heavies can foul the hydrogenation catalyst.

In some embodiments, the system includes an activated carbon unit constructed to remove impurities from a bioderived 1,3-BG containing product stream. In some embodiments, the activated carbon unit can receive a crude bioderived 1,3-BG mixture or partially purified bioderived 1,3-BG and generate an activated carbon-treated crude bioderived 1,3-BG mixture or partially purified bioderived 1,3-BG that can be fed into a polishing column or a dewatering column. In some embodiments, the polishing column is an ion exchange chromatography column. In some embodiments, the activated carbon unit can receive a crude bioderived 1,3-BG mixture or partially purified bioderived 1,3-BG of reduced salt content and generate an activated carbon-treated crude bioderived 1,3-BG mixture or partially purified bioderived 1,3-BG that can be fed into a dewatering column. In some embodiments, the activated carbon unit can receive a first bioderived 1,3-BG containing product stream and generate an activated carbon-treated first bioderived 1,3-BG containing product stream that can be fed into a first distillation column. In some embodiments, the activated carbon unit reactor can receive a second bioderived 1,3-BG containing product stream and generate an activated carbon-treated bioderived 1,3-BG containing product stream that can be fed into a second distillation column. In some embodiments, the activated carbon unit can receive a third bioderived 1,3-BG containing product stream and generate an activated carbon-treated third bioderived 1,3-BG containing product stream that can be fed into a third distillation column. In some embodiments, the activated carbon unit can receive a fourth bioderived 1,3-BG containing product stream and generate an activated carbon-treated fourth bioderived 1,3-BG containing product stream. In some embodiments, the fourth bioderived 1,3-BG containing product stream includes a purified bioderived 1,3-BG product. In some embodiments, the first, second, third, or fourth bioderived 1,3-BG containing product stream consists essentially of a bioderived 1,3-BG provided herein.

In some embodiments, the system includes a sodium borohydride (NaBH₄) addition device. In some embodiments, the NaBH₄ addition device can receive a crude bioderived 1,3-BG mixture or partially purified bioderived 1,3-BG and generate a NaBH₄-treated crude bioderived 1,3-BG mixture or partially purified bioderived 1,3-BG that can be fed into a polishing column or a dewatering column. In some embodiments, the polishing column is an ion exchange chromatography column, or includes activated carbon. In some embodiments, the NaBH₄ addition device can receive a crude bioderived 1,3-BG mixture or partially purified bioderived 1,3-BG of reduced salt content and generate a NaBH₄-treated crude bioderived 1,3-BG mixture or partially purified bioderived 1,3-BG that can be fed into a dewatering column. In some embodiments, the NaBH₄ addition device can receive a first bioderived 1,3-BG containing product stream and generate a NaBH₄-treated first bioderived 1,3-BG containing product stream that can be fed into a first distillation column. In some embodiments, the NaBH₄ addition device can receive a second bioderived 1,3-BG containing product stream and generate a NaBH₄-treated second bioderived 1,3-BG containing product stream that can be fed into a second distillation column. In some embodiments, the NaBH₄ addition device can receive a third bioderived 1,3-BG containing product stream and generate a NaBH₄-treated third bioderived 1,3-BG containing product stream that can be fed into a third distillation column. In some embodiments, the NaBH₄ addition device can receive a fourth bioderived 1,3-BG containing product stream and generate NaBH₄-treated fourth bioderived 1,3-BG containing product stream. In some embodiments, the fourth bioderived 1,3-BG containing product stream includes a purified bioderived 1,3-BG product. In some embodiments, the first, second, third, or fourth bioderived 1,3-BG containing product stream consists essentially of a bioderived 1,3-BG provided herein.

In some embodiments, the system includes a wiped-film evaporator (WFE) receiving the first stream of materials with boiling points higher than 1,3-BG and generating a distillate, whereas the distillate is fed to the first distillation column. In some embodiments, the system includes a WFE receiving the second stream of materials with boiling points higher than 1,3-BG and generating a distillate, whereas the distillate is fed to the third distillation column. In some embodiments, the system includes a WFE receiving the first stream of materials with boiling points higher than 1,3-BG and generating a distillate, whereas the distillate is fed to the first distillation column, and the system includes a WFE receiving the second stream of materials with boiling points higher than 1,3-BG and generating a distillate, whereas the distillate is fed to the third distillation column.

In some embodiments, the system includes one or more reboilers. Reboilers are heat exchangers that are typically used to provide heat to the bottom of industrial distillation columns. Reboilers can boil the liquid from the bottom of a distillation column to generate vapors which are returned to the column to drive the distillation separation, e.g., of bioderived 1,3-BG. The heat supplied to a distillation column by a reboiler at the bottom of the column is generally removed by a condenser at the top of the column. Reboilers can include, e.g., a kettle reboiler, a thermosyphon reboiler, a fired reboiler, or a forced circulation reboiler.

In some embodiments, the system includes a reboiler receiving liquid from a dewatering column generating vapor, whereby the vapor is returned to the dewatering column. In some embodiments, the system includes a reboiler receiving liquid from a first, second, or third distillation column, or combinations thereof, generating vapor, whereby the vapor is returned to the first, second, or third distillation column, or combinations thereof. In some embodiments, the system includes a reboiler receiving liquid from the dewatering column generating vapor, whereby the vapor is returned to the dewatering column. In some embodiments, the system includes a reboiler receiving liquid from a dewatering column generating vapor, whereby the vapor is returned to the dewatering column, and the system includes a reboiler receiving liquid from a first, second, or third distillation column, or combinations thereof, generating vapor, whereby the vapor is returned to the first, second, or third distillation column, or combinations thereof.

In some embodiments, the reboiler is used to add a reagent, such as a base, to the system or a process using the system.

In some embodiments, the purified bioderived 1,3-BG product produced by a system provided herein consists essentially of a bioderived 1,3-BG provided herein.

In another aspect, provided herein is bioderived 1,3-BG produced by a system provided herein. In some embodiments, the bioderived 1,3-BG produced by a system provided herein is a bioderived 1,3-BG provided herein.

In some embodiments, the bioderived 1,3-BG has a chiral purity of 55% or more, or 95% or more, or any other chiral purity disclosed herein. For example, a crude bioderived 1,3-BG mixture or partially purified bioderived 1,3-BG, e.g., such as input into a distillation system such as described with reference to FIGS. 15A-15C, can include bioderived 1,3-BG having a chiral purity of 55% or more.

In some embodiments, a purified bioderived 1,3-BG product has a chemical purity of 99.0% or more, or 99.5% or more, or any other chemical purity disclosed herein. For example, the purified bioderived 1,3-BG product output from a distillation system such as described with reference to FIGS. 15A-15C, can include bioderived 1,3-BG having a chemical purity of 99.0% or more. Additionally, in some embodiments, purified bioderived 1,3-BG product output can include 1,3-BG having a chiral purity of 55% or more.

An example of a distillation system provided herein is depicted in FIG. 15A. The crude bioderived 1,3-BG mixture or partially purified bioderived 1,3-BG 500 is fed to the dewatering column 510, where light materials 512 (materials with boiling points lower than 1,3-BG, such as water) are removed from the top of the first column 510. A bioderived 1,3-BG-containing product stream 514 exits the bottom of the first column and is fed to a first distillation column 520. Heavy materials 524 (materials with boiling points higher than 1,3-BG) are removed from the bottom of the first distillation column 520, and a bioderived 1,3 BG-containing product stream 522 exits from the top of the first distillation column 520. The heavy material 524 can optionally be fed to a wiped-film evaporator (WFE) 525, where a WFE distillate 542 and heavy material are produced. The WFE distillate 542 optionally is fed to the first distillation column 520. The bioderived 1,3-BG-containing product stream 522 is fed to a second distillation column 530. Distillation column 530 removes light materials 532 from the top of the column 530 and a third bioderived 1,3-BG-containing product stream 534 from the bottom of column 530. The third bioderived 1,3-BG containing product stream (1,3-BDO-containing product stream) 534 is fed to a third distillation column 550. The purified bioderived 1,3-BG (1,3-BDO) product 552 is collected from the top of column 550, and heavy materials 554 exit from the bottom of column 550.

An example depicted in FIG. 15B adds an alkali reactor 560′ to the system of FIG. 15A. For example, the crude bioderived 1,3-BG mixture or partially purified bioderived 1,3-BG 500′ is fed to the dewatering column 510′, where light materials 512′ (materials with boiling points lower than 1,3-BG, such as water) are removed from the top of the first column 510′. A bioderived 1,3-BG-containing product stream 514′ exits the bottom of the first column and is fed to a first distillation column 520′. Heavy materials 524′ (materials with boiling points higher than 1,3-BG) are removed from the bottom of the first distillation column 520′, and a bioderived 1,3 BG-containing product stream 522′ exits from the top of the first distillation column 520′. The heavy material 524′ optionally can be fed to a WFE 525′, where a WFE distillate 542′ and heavy material are produced, and the WFE distillate 542′ optionally is fed to the first distillation column. The bioderived 1,3-BG-containing product stream 522′ is fed to the alkali reactor 560′, which sends the stream 562″ to the second distillation column 530′. Distillation column 530′ removes light materials 532′ from the top of the column 530′ and a third bioderived 1,3-BG-containing product stream 534′ from the bottom of column 530′. The third bioderived 1,3-BG containing product stream (1,3-BDO-containing product stream) 534′ is fed to a third distillation column 550′. The purified bioderived 1,3-BG (1,3-BDO) product 552′ is collected from the top of column 550′, and heavy materials 554′ exit from the bottom of column 550′.

An example depicted in FIG. 15C adds an activated carbon unit 570″ to the system of FIG. 15A. For example, the crude bioderived 1,3-BG mixture or partially purified bioderived 1,3-BG 500″ is fed to the dewatering column 510″, where light materials 512″ (materials with boiling points lower than 1,3-BG, such as water) are removed from the top of the first column 510″. A bioderived 1,3-BG-containing product stream 514″ exits the bottom of the first column and is fed to a first distillation column 520″. Heavy materials 524″ (materials with boiling points higher than 1,3-BG) are removed from the bottom of the first distillation column 520″, and a bioderived 1,3 BG-containing product stream 522″ exits from the top of the first distillation column 520″. The heavy material 524″ optionally can be fed to a WFE 525″, where a WFE distillate 542″ and heavy material are produced, and the WFE distillate 542″ optionally is fed to the first distillation column. The bioderived 1,3-BG-containing product stream 522″ optionally is fed to an alkali reactor (not specifically illustrated in FIG. 15C), which sends the stream to the second distillation column 530″ in a manner such as described with reference to FIG. 15B. Distillation column 530″ removes light materials 532″ from the top of the column 530″ and a third bioderived 1,3-BG-containing product stream 534″ from the bottom of column 530″. The third bioderived 1,3-BG containing product stream (1,3-BDO-containing product stream) 534″ is fed to a third distillation column 550″. The purified bioderived 1,3-BG (1,3-BDO) product 552″ is collected from the top of column 550″, and heavy materials 554″ exit from the bottom of column 550″. The purified bioderived 1,3-BG (1,3-BDO) product 552″ is fed to the activated carbon unit 570″, which generates an activated carbon treated product 572″.

In some embodiments, the carbon feedstock and other cellular uptake sources such as phosphate, ammonia, sulfate, chloride and other halogens can be chosen to alter the isotopic distribution of the atoms present in bioderived 1,3-BG (1,3-BDO), or a downstream product related thereto such as an ester or amide thereof, or any bioderived 1,3-BG (1,3-BDO) pathway intermediate. The various carbon feedstock and other uptake sources enumerated above will be referred to herein, collectively, as “uptake sources.” Uptake sources can provide isotopic enrichment for any atom present in the product bioderived 1,3-BG (1,3-BDO), or a downstream product related thereto such as an ester or amide thereof, or bioderived 1,3-BG (1,3-BDO) pathway intermediate, or for side products generated in reactions diverging away from a bioderived 1,3-BG (1,3-BDO) pathway. Isotopic enrichment can be achieved for any target atom including, for example, carbon, hydrogen, oxygen, nitrogen, sulfur, phosphorus, chloride or other halogens.

In some embodiments, the uptake sources can be selected to alter the carbon-12, carbon-13, and carbon-14 ratios. In some embodiments, the uptake sources can be selected to alter the oxygen-16, oxygen-17, and oxygen-18 ratios. In some embodiments, the uptake sources can be selected to alter the hydrogen, deuterium, and tritium ratios. In some embodiments, the uptake sources can be selected to alter the nitrogen-14 and nitrogen-15 ratios. In some embodiments, the uptake sources can be selected to alter the sulfur-32, sulfur-33, sulfur-34, and sulfur-35 ratios. In some embodiments, the uptake sources can be selected to alter the phosphorus-31, phosphorus-32, and phosphorus-33 ratios. In some embodiments, the uptake sources can be selected to alter the chlorine-35, chlorine-36, and chlorine-37 ratios.

In some embodiments, the isotopic ratio of a target atom can be varied to a desired ratio by selecting one or more uptake sources. An uptake source can be derived from a natural source, as found in nature, or from a man-made source, and one skilled in the art can select a natural source, a man-made source, or a combination thereof, to achieve a desired isotopic ratio of a target atom. An example of a man-made uptake source includes, for example, an uptake source that is at least partially derived from a chemical synthetic reaction. Such isotopically enriched uptake sources can be purchased commercially or prepared in the laboratory and/or optionally mixed with a natural source of the uptake source to achieve a desired isotopic ratio. In some embodiments, a target atom isotopic ratio of an uptake source can be achieved by selecting a desired origin of the uptake source as found in nature. For example, as discussed herein, a natural source can be a biobased source derived from or synthesized by a biological organism or a source such as petroleum-based products or the atmosphere. In some such embodiments, a source of carbon, for example, can be selected from a fossil fuel-derived carbon source, which can be relatively depleted of carbon-14, or an environmental or atmospheric carbon source, such as CO₂, which can possess a larger amount of carbon-14 than its petroleum-derived counterpart.

The unstable carbon isotope carbon-14 or radiocarbon makes up for roughly 1 in 10¹² carbon atoms in the earth's atmosphere and has a half-life of about 5700 years. The stock of carbon is replenished in the upper atmosphere by a nuclear reaction involving cosmic rays and ordinary nitrogen (¹⁴N). Fossil fuels contain no carbon-14, as it decayed long ago. Burning of fossil fuels lowers the atmospheric carbon-14 fraction, the so-called “Suess effect”.

Methods of determining the isotopic ratios of atoms in a compound are well known to those skilled in the art. Isotopic enrichment is readily assessed by mass spectrometry using techniques known in the art such as accelerated mass spectrometry (AMS), Stable Isotope Ratio Mass Spectrometry (SIRMS) and Site-Specific Natural Isotopic Fractionation by Nuclear Magnetic Resonance (SNIF-NMR). Such mass spectral techniques can be integrated with separation techniques such as liquid chromatography (LC), high performance liquid chromatography (HPLC) and/or gas chromatography, and the like.

In the case of carbon, ASTM D6866 was developed in the United States as a standardized analytical method for determining the biobased content of solid, liquid, and gaseous samples using radiocarbon dating by the American Society for Testing and Materials (ASTM) International. The standard is based on the use of radiocarbon dating for the determination of a product's biobased content. ASTM D6866 was first published in 2004, and the current active version of the standard is ASTM D6866-11 (effective Apr. 1, 2011). Radiocarbon dating techniques are well known to those skilled in the art, including those described herein.

The biobased content of a compound is estimated by the ratio of carbon-14 (¹⁴C) to carbon-12 (¹²C). Specifically, the Fraction Modern (Fm) is computed from the expression: Fm=(S−B)/(M−B), where B, S and M represent the ¹⁴C/¹²C ratios of the blank, the sample and the modern reference, respectively. Fraction Modern is a measurement of the deviation of the ¹⁴C/¹²C ratio of a sample from “Modern.” Modern is defined as 95% of the radiocarbon concentration (in AD 1950) of National Bureau of Standards (NBS) Oxalic Acid I (i.e., standard reference materials (SRM) 4990b) normalized to δ¹³C_{VPDB}=−19 per mil (Olsson, The use of Oxalic acid as a Standard, in Radiocarbon Variations and Absolute Chronology, Nobel Symposium, 12th Proc., John Wiley & Sons, New York (1970), the entire contents of which are incorporated by reference herein). Mass spectrometry results, for example, measured by ASM, are calculated using the internationally agreed upon definition of 0.95 times the specific activity of NBS Oxalic Acid I (SRM 4990b) normalized to δ¹³C_{VPDB}=−19 per mil. This is equivalent to an absolute (AD 1950) ¹⁴C/¹²C ratio of 1.176±0.010×10⁻¹² (Karlen et al., Arkiv Geofysik, 4:465-471 (1968), the entire contents of which are incorporated by reference herein). The standard calculations take into account the differential uptake of one isotope with respect to another, for example, the preferential uptake in biological systems of C¹² over C¹³ over C¹⁴, and these corrections are reflected as a Fm corrected for δ¹³.

An oxalic acid standard (SRm 4990b or HOx 1) was made from a crop of 1955 sugar beet. Although there were 1000 lbs made, this oxalic acid standard is no longer commercially available. The Oxalic Acid II standard (HOx 2; N.I.S.T designation SRM 4990 C) was made from a crop of 1977 French beet molasses. In the early 1980's, a group of 12 laboratories measured the ratios of the two standards. The ratio of the activity of Oxalic acid II to 1 is 1.2933±0.001 (the weighted mean). The isotopic ratio of HOx II is −17.8 per mil. ASTM D6866-11 suggests use of the available Oxalic Acid II standard SRM 4990 C (Hox2) for the modern standard (see discussion of original vs. currently available oxalic acid standards in Mann, Radiocarbon, 25(2):519-527 (1983), the entire contents of which are incorporated by reference herein). A Fm=0% represents the entire lack of carbon-14 atoms in a material, thus indicating a fossil (for example, petroleum based) carbon source. A Fm=100%, after correction for the post-1950 injection of carbon-14 into the atmosphere from nuclear bomb testing, indicates an entirely modern carbon source. As described herein, such a “modern” source includes biobased sources.

As described in ASTM D6866, the percent modern carbon (pMC) can be greater than 100% because of the continuing but diminishing effects of the 1950s nuclear testing programs, which resulted in a considerable enrichment of carbon-14 in the atmosphere as described in ASTM D6866-11. Because all sample carbon-14 activities are referenced to a “pre-bomb” standard, and because nearly all new biobased products are produced in a post-bomb environment, all pMC values (after correction for isotopic fraction) must be multiplied by 0.95 (as of 2010) to better reflect the true biobased content of the sample. A biobased content that is greater than 103% suggests that either an analytical error has occurred, or that the source of biobased carbon is more than several years old.

ASTM D6866 quantifies the biobased content relative to the material's total organic content and does not consider the inorganic carbon and other non-carbon containing substances present. For example, a product that is 50% starch-based material and 50% water would be considered to have a Biobased Content=100% (50% organic content that is 100% biobased) based on ASTM D6866. In another example, a product that is 50% starch-based material, 25% petroleum-based, and 25% water would have a Biobased Content=66.7% (75% organic content but only 50% of the product is biobased). In another example, a product that is 50% organic carbon and is a petroleum-based product would be considered to have a Biobased Content=0% (50% organic carbon but from fossil sources). Thus, based on the well known methods and known standards for determining the biobased content of a compound or material, one skilled in the art can readily determine the biobased content of a compound or material and/or prepared downstream products that utilize a compound or material provided herein having a desired biobased content.

Applications of carbon-14 dating techniques to quantify bio-based content of materials are known in the art (Currie et al., Nuclear Instruments and Methods in Physics Research B, 172:281-287 (2000), the entire contents of which are incorporated by reference herein). For example, carbon-14 dating has been used to quantify bio-based content in terephthalate-containing materials (Colonna et al., Green Chemistry, 13:2543-2548 (2011), the entire contents of which are incorporated by reference herein). Notably, polypropylene terephthalate (PPT) polymers derived from renewable 1,3-propanediol and petroleum-derived terephthalic acid resulted in Fm values near 30% (i.e., since 3/11 of the polymeric carbon derives from renewable 1,3-propanediol and 8/11 from the fossil end member terephthalic acid) (Currie et al., supra, 2000). In contrast, polybutylene terephthalate polymer derived from both renewable 1,4-butanediol and renewable terephthalic acid resulted in bio-based content exceeding 90% (Colonna et al., supra, 2011).

Accordingly, in some embodiments, the present disclosure provides bioderived 1,3-BG (1,3-BDO) or a downstream product related thereto such as an ester or amide thereof, or a bioderived 1,3-BG (1,3-BDO) pathway intermediate, produced by a suitable cell, that has a carbon-12, carbon-13, and carbon-14 ratio that reflects an atmospheric carbon, also referred to as environmental carbon, uptake source. For example, in some aspects the bioderived 1,3-BG (1,3-BDO), or a downstream product related thereto such as an ester or amide thereof, or a bioderived 1,3-BG (1,3-BDO) pathway intermediate can have an Fm value of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or as much as 100%. In some such embodiments, the uptake source is CO₂. In some embodiments, the present compositions, systems, and methods provide bioderived 1,3-BG (1,3-BDO), or a downstream product related thereto such as an ester or amide thereof, or a bioderived 1,3-BG (1,3-BDO) pathway intermediate that has a carbon-12, carbon-13, and carbon-14 ratio that reflects petroleum-based carbon uptake source. In this aspect, the bioderived 1,3-BG (1,3-BDO), or a downstream product related thereto such as an ester or amide thereof, or a bioderived 1,3-BG (1,3-BDO) pathway intermediate can have an Fm value of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2% or less than 1%. In some embodiments, the present compositions, systems, and methods provide bioderived 1,3-BG (1,3-BDO), or a downstream product related thereto such as an ester or amide thereof, or a bioderived 1,3-BG (1,3-BDO) pathway intermediate that has a carbon-12, carbon-13, and carbon-14 ratio that is obtained by a combination of an atmospheric carbon uptake source with a petroleum-based uptake source. Using such a combination of uptake sources is one way by which the carbon-12, carbon-13, and carbon-14 ratio can be varied, and the respective ratios would reflect the proportions of the uptake sources.

Further, the present compositions, systems, and methods relate to the biologically produced bioderived 1,3-BG (1,3-BDO), or a downstream product related thereto such as an ester or amide thereof, or bioderived 1,3-BG (1,3-BDO) pathway intermediate as disclosed herein, and to the products derived therefrom, wherein the bioderived 1,3-BG (1,3-BDO), or a downstream product related thereto such as an ester or amide thereof, or a bioderived 1,3-BG (1,3-BDO) pathway intermediate has a carbon-12, carbon-13, and carbon-14 isotope ratio of about the same value as the CO₂ that occurs in the environment. For example, in some aspects the present compositions, systems, and methods provide bioderived 1,3-BG (1,3-BDO), or a downstream product related thereto such as an ester or amide thereof, or a bioderived 1,3-BG (1,3-BDO) intermediate having a carbon-12 versus carbon-13 versus carbon-14 isotope ratio of about the same value as the CO₂ that occurs in the environment, or any of the other ratios disclosed herein. It is understood, as disclosed herein, that a product can have a carbon-12 versus carbon-13 versus carbon-14 isotope ratio of about the same value as the CO₂ that occurs in the environment, or any of the ratios disclosed herein, wherein the product is generated from bioderived 1,3-BG (1,3-BDO), or a downstream product related thereto such as an ester or amide thereof, or a bioderived 1,3-BG (1,3-BDO) pathway intermediate as disclosed herein, wherein the bioderived product is chemically modified to generate a final product. Methods of chemically modifying a bioderived product of bioderived 1,3-BG (1,3-BDO), or a downstream product related thereto such as an ester or amide thereof, or an intermediate of a bioderived 1,3-BG (1,3-BDO), to generate a desired product are well known to those skilled in the art, as described herein.

The present compositions, systems, and methods further provide plastics, elastic fibers, polyurethanes, polyesters, including polyhydroxyalkanoates, nylons, organic solvents, polyurethane resins, polyester resins, hypoglycaemic agents, butadiene and/or butadiene-based products, which can be based on bioderived 1,3-BG (1,3-BDO), or a downstream product related thereto such as an ester or amide thereof, and plastics, elastic fibers, polyurethanes, polyesters, including polyhydroxyalkanoates such as poly-4-hydroxybutyrate (P4HB) or co-polymers thereof, poly(tetramethylene ether)glycol (PTMEG) (also referred to as PTMO, polytetramethylene oxide), polybutylene terephthalate (PBT), and polyurethane-polyurea copolymers, referred to as spandex, elastane or Lycra™, nylons, and the like, which can be based on bioderived 1,3-BG (1,3-BDO), or a downstream product related thereto such as an ester or amide thereof, having a carbon-12 versus carbon-13 versus carbon-14 isotope ratio of about the same value as the CO₂ that occurs in the environment, wherein the plastics, elastic fibers, polyurethanes, polyesters, including polyhydroxyalkanoates such as poly-4-hydroxybutyrate (P4HB) or co-polymers thereof, poly(tetramethylene ether)glycol (PTMEG) (also referred to as PTMO, polytetramethylene oxide), polybutylene terephthalate (PBT), and polyurethane-polyurea copolymers, referred to as spandex, elastane or Lycra™, nylons, organic solvents, polyurethane resins, polyester resins, hypoglycaemic agents, butadiene, and/or butadiene-based products are generated directly from or in combination with bioderived 1,3-BG (1,3-BDO), or a downstream product related thereto such as an ester or amide thereof, or a bioderived 1,3-BG (1,3-BDO) pathway intermediate as disclosed herein.

Bioderived 1,3-BG (1,3-BDO) can be reacted with an acid, either in vivo or in vitro, to convert to an ester using, for example, a lipase. Such esters can have nutraceutical, pharmaceutical and food uses, and are advantaged when R-form of 1,3-BG (1,3-BDO) is used since that is the form (compared to S-form or the racemic mixture) best utilized by both animals and humans as an energy source (e.g., a ketone ester, such as (R)-3-hydroxybutyl-R-1,3-butanediol monoester (which has Generally Recognized As Safe (GRAS) approval in the United States) and (R)-3-hydroxybutyrate glycerol monoester or diester). The ketone esters can be delievered orally, and the ester releases R-1,3-butanediol that is used by the body (see, for example, WO2013150153, the entire contents of which are incorporated by reference herein). Thus the present compositions, systems, and methods are particularly useful to provide an improved enzymatic route and microorganism to provide an improved composition of bioderived 1,3-BG (1,3-BDO), namely or such as R-1,3-butanediol, highly enriched or essentially enantiomerically pure, and further having improved purity qualities with respect to by-products.

Bioderived 1,3-BG (1,3-BDO) has or can have further food related uses including use directly as a food source, a food ingredient, a flavoring agent, a solvent or solubilizer for flavoring agents, a stabilizer, an emulsifier, and an anti-microbial agent and preservative. Bioderived 1,3-BG (1,3-BDO) is or can be used in the pharmaceutical industry as a parenteral drug solvent. Bioderived 1,3-BG (1,3-BDO) finds or can find use in cosmetics as an ingredient that is an emollient, a humectant, that prevents crystallization of insoluble ingredients, a solubilizer for less-water-soluble ingredients such as fragrances, and as an anti-microbial agent and preservative. For example, it can be used as a humectant, especially in hair sprays and setting lotions; it reduces or can reduce loss of aromas from essential oils, preserves against spoilage by microorganisms, and is used or can be used as a solvent for benzoates. Bioderived 1,3-BG (1,3-BDO) can be used at concentrations from 0.1% to 50%, and even less than 0.1% and even more than 50%. It is or can be used in hair and bath products, eye and facial makeup, fragrances, personal cleanliness products, and shaving and skin care preparations (see, for example, the Cosmetic Ingredient Review board's report: “Final Report on the Safety Assessment of Butylene Glycol, Hexylene Glycol, Ethoxydiglycol, and Dipropylene Glycol”, Journal of the American College of Toxicology, Volume 4, Number 5, 1985, which is incorporated herein by reference in its entirety). This report provides specific uses and concentrations of 1,3-BG (1,3-BDO) in cosmetics; see for examples the report's Table 2 therein entitled “Product Formulation Data”.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference in their entireties, to the same extent as if each independent patent and publication was specifically and individually indicated to be incorporated by reference in its entirety.

The following examples are provided by way of illustration, not limitation.

### Example 1

### Laboratory-Scale Production and Purification of Bio-BG

A fermentation broth enriched in bioderived 1,3-BG was produced using strain and following a protocol as described, e.g., in WO 2010/127319 A2 and WO 2011/071682 A1, the entire contents of each of which are incorporated by reference herein. In brief, an exemplary or a preferred microbial route to bio-BG is described in WIPO patent publication WO2010127319A2, see especially routes comprising a 3-hydroxybutyryl-CoA dehydrogenase, as for example the pathway from acetoacetyl-CoA to 1,3-butanediol of FIG. 2 therein that includes step H. In one embodiment the 3-hydroxybutyryl-CoA dehydrogenase may have and can be modified to have specificity for an R enantiomer. Reference is also made to the following provisional applications, which are incorporated herein by reference in their entireties: (1) U.S. Provisional Application No. 62/480,208 entitled “3-HYDROXYBUTYRYL-COA DEHYDROGENASE VARIANTS AND METHODS OF USE,” filed Mar. 31, 2017 (Attorney Docket No. 12956-409-888); (2) U.S. Provisional Application No. 62/480,194 entitled, “ALDEHYDE DEHYDROGENASE VARIANTS AND METHODS OF USE,” filed Mar. 31, 2017 (Attorney Docket No. 12956-408-888); (3) International Patent Application No. ______ entitled “3-HYDROXYBUTYRYL-COA DEHYDROGENASE VARIANTS AND METHODS OF USE,” filed on even date herewith (Attorney Docket No. 12956-409-228); and (4) International Patent Application No. ______ entitled, “ALDEHYDE DEHYDROGENASE VARIANTS AND METHODS OF USE,” filed on even date herewith (Attorney Docket No. 12956-408-228).

Bioderived 1,3-BG was subsequently purified from the fermentation broth using a sequence of (1) microfiltration, (2) nanofitration, (3) ion-exchange chromatography, (4) evaporation of water, and (5) polishing ion-exchange to produce a crude mix containing bioderived 1,3-BG. The crude mix was then fed into a dewatering distillation column to produce a 1,3-BG-containing product stream that was fed into a 2 L batch distillation column to produce a bioderived 1,3-BG product. The batch distillation column was a randomly packed column of 1″ diameter, about 2 ft tall, and had a condenser and reflux control attached directly on top of the column.

### Batch-Distillation at High Reflux Rates can Produce Highly Pure Bioderived 1,3-BG

This Example demonstrates that a batch distillation process, e.g., using a laboratory scale distillation system as described above, can yield bio-BG of the highest purity even in the absence of additional purification steps involving, e.g., active carbon treatments, hydrogenation, base addition, or borohydride treatments. Exemplary results are shown in Table 2 for a distillation process involving dewatering/heavies (DW/HV) distillation at a 3:1 reflux ratio followed by lights/1,3-BG (LT/BG) distillation at a 3:1 reflux ratio. Highly pure bioderived 1,3-BG fractions were obtained having a purity of 99.9% on a dry-basis and 4-hydroxy-2-butanone and 3-butanal levels of below 50 ppm.

It is believed that further improvements in the purity and odor of bioderived 1,3-BG can be achieved using a continuous distillation process. Especially continuous distillation processes involving deep vacuum and high reflux ratios are believed to be useful for odor reduction of bio-1,3-BG. Without wishing to be bound by theory, it is believed that under the conditions of such a process, degradation of 4-hydroxy-2-butanone (4-OH-2-butanone) and 3-hydroxy-butanal (3-OH-butanal) to potent odor byproducts, such as MVK and Cr-Ald, can be reduced or avoided.

TABLE 2
Results of a batch distillation process for bioderived 1,3-BG involving dewatering/heavies distillation at a 3:1 reflux
ratio followed by lights/1,3-BG distillation at a 3:1 reflux ratio.

| | [1,3-BG | [1,3-BG | | | | | | | | | | |
|---|---|---|---|---|---|---|---|---|---|---|---|---|
| | (BDO) | (BDO) | | [3-OH- | [4-OH-2- | | [n- | [1-Hydroxy- | | | | [3-Hydroxy- |
| | Heavies] | Lights] | [Purity] | Butanal] | butanone] | [IPA] | But] | 2-Propanone] | [12PDO] | 13PDO] | 23BDO] | 2-butanone] |
| Description | % | % | % | ppm | ppm | ppm | ppm | ppm | ppm | ppm | ppm | ppm |
| Evap Product | 3.7 | 0.3 | 96 | | 10376.91 | — | — | 71.99 | 3066.11 | — | 3052.38 | 23.9 |
| PIX Feed | 3.5 | 0.3 | 96.1 | | 8003.77 | — | — | 47.15 | 2981.72 | 111.1 | 2763.35 | 12.3 |
| PIX Product | 4.8 | 0.4 | 94.8 | | 7204.66 | — | — | 27.10 | 2,695.22 | 119.04 | 2,458.34 | 15.22 |
| Evap Prod | 4.8 | 0.4 | 94.8 | | 8003.77 | — | — | — | 2,981.72 | 111.10 | 2,763.35 | — |
| after PIX | | | | | | | | | | | | |
| HV Product | 0.4 | 0.1 | 99.5 | 248.55 | 145.57 | — | — | 7.47 | 159.6 | — | 34.36 | 4.04 |
| Lights Dist 1 | 0.7 | 1.1 | 99.2 | 1,704.51 | 1,155.98 | — | — | 39.22 | 692.07 | — | 359.37 | 8.54 |
| BG Dist 1 | 0.9 | 1.5 | 97.6 | 335.76 | 331.49 | — | — | 11.89 | 297.42 | — | 72.05 | 2.99 |
| BG Dist 2 | 0.5 | 0.1 | 99.4 | 72.43 | 69.1 | — | — | 5.23 | 65.99 | — | 1.59 | 1.25 |
| BG Dist 3 | 0.2 | 0.01 | 99.7 | 50.99 | 26.8 | — | — | 2.53 | 7.43 | — | — | 1.53 |
| BG Dist 4 | 0.2 | 0.01 | 99.8 | 62.04 | 28.42 | — | — | 1.84 | 8.57 | — | — | 0 |
| BG Dist 5 | 0.15 | 0 | 99.9 | 57.43 | 21.5 | — | — | 1.58 | 3.93 | — | — | 0 |
| BG Dist 6 | 0.1 | 0.02 | 99.9 | 26.76 | 23.86 | — | — | 2.24 | 5.29 | — | — | 0 |
| BG Dist 7 | 0.08 | 0 | 99.9 | 49.14 | 27.67 | — | — | 2.14 | 7.19 | — | — | 0.57 |
| BG Dist 8 | 0.1 | 0.01 | 99.9 | 48.09 | 25.2 | — | — | — | 3.71 | — | — | 0 |
| BG Dist 9 | 0.1 | 0.01 | 99.9 | 62.7 | 19 | — | — | 2.83 | 1.4 | — | — | 0.62 |
| BG Bottoms | 0.1 | — | 99.9 | 53.75 | 14.04 | — | — | 2.32 | — | — | — | — |

3-OH-Butanal: 3-hydroxy-butanal;
4-OH-2-Butanone: 4-hydroxy-2-butanone;
IPA: isopropyl-alcohol;
n-But: n-butanol;
12PDO: 1,2-propanediol;
13PDO: 1,3-propanediol;
23BDO (2,3-BDO): 2,3-butanediol

### Example 2

### GC-MS Analysis and Comparison of Bio-BG and Petro-BG

Comparative purity evaluations of bio-BG and petro-BG samples were conducted using gas-chromatography/mass spectrometry (GC-MS) analysis. Representative bio-BG samples were obtained at laboratory scale as described in Example 1. Representative industrial-grade and cosmetic-grade petro-BG reference samples are commercially available, e.g., from Oxea Corp., Bay City, TX. Compounds having GC retention times shorter than 1,3-BG are referred to herein as “lights.” Compounds having GC retention times longer than 1,3-BG are herein referred to as “heavies.”

In brief, 3-hydroxy-butanal (3OH-butanal) and 4-hydroxy-2-butanone (40H-2-butanone) were identified or believed to be identified as two bio-BG specific compounds that were present at substantially higher levels in bio-BG samples (˜1,000 ppm). 3-hydroxy-butanal and 4-hydroxy-2-butanone were either not detectable by GC-MS in industrial-grade petro-BG or cosmetic-grade petro-BG samples, or were present at substantially lower levels (e.g., ˜100-1,000-fold lower levels) in industrial-grade or cosmetic-grade petro-BG relative to bio-BG.

Two additional bio-BG specific compounds were identified as heavies in bio-BG samples and are referred to herein as “compound 7” and “compound 9.” Compounds 7 and 9 were either undetectable by GC-MS in industrial-grade or cosmetic-grade petro-BG, or present at substantially lower levels (e.g., ˜100-1,000-fold lower levels) in industrial-grade or cosmetic-grade petro-BG relative to bio-BG. Although proposed structures for compounds 7 and 9 are provided elsewhere herein, such proposed structures are not intended to be limiting.

Generally, industrial-grade and cosmetic-grade petro-BG samples were found to have greater numbers and higher levels of “heavies” impurities compared to bio-BG samples, such as bio-BG samples of Example 1, as determined by GC-MS.

1,3-BG samples were diluted 2-fold (DF2) or 20-fold (DF20) in acetonitrile and subjected to GC-MS analysis. DF2 samples were used to quantify known impurities in the samples based on a multi-level external standard calibration, as described below. DF20 samples were used to determine (area) % purity of 1,3-BG “lights” and “heavies” based on total ion current (TIC) peak areas.

An Agilent gas chromatograph 6890N was used for the 1,3-BG analysis, interfaced to a mass-selective detector (MSD) 5973N, and operated in electron impact ionization (EI) mode. 0.5 μL of 1,3-BG sample, diluted 2-fold or 20-fold with acetonitrile, was introduced in a split injection mode at 50:1 split ratio and at an injection port temperature of 250° C. Helium was used as a carrier gas, and a constant flow rate of the carrier gas was maintained at 1.5 mL/min. The following fast GC temperature program was developed to analyze 1,3-BG purity on an HP-INNOWax™ column (Agilent Technologies, Santa Clara, CA): the oven was initially held at 50° C. for 3 min, followed by an increase to 250° C. at 15° C./min, and held for 5 min (total run time is 21.33 min). The MS interface transfer line was maintained at 280° C. Data are acquired using a 25-500 m/z mass-range scan.

Typical retention times (RT) on an HP-INNOWax™ capillary column (30 m×0.25 mm×0.25 μm (Agilent)) were established for all known heavies or lights compounds in 1,3-BG samples by injecting neat control compounds.

An external standard calibration was developed for identified heavies or lights compounds, such as 3-hydroxy-butanal, 4-hydroxy-2-butanone and others. Standard calibration included a series of 6 reference compound concentrations ranging from 5 to 1000 ppm of the control compound. Total ion current (TIC) and/or extracted ion current (XIC) chromatograms, based on characteristic target ions for each compound of interest, were used for quantitation. In addition, qualifier ions were selected from the mass spectrum of each target compound. The relative signal intensities of qualifier ions to target ions was determined to confirm the identity of the target compound. Quantitation of test compounds was performed based on control compound standard curves using a quadratic fit.

Calculations for % purity represent GC purity based on GC peak areas. Compounds having retention times shorter than 1,3-BG (RT˜11.85 min) are referred to as “lights,” compounds having retention times longer than 1,3-BG are referred to as “heavies.”

FIG. 1 shows an overlay of exemplary GC-MS chromatograms (total ion currents, TICs) of a bio-BG sample and industrial-grade petro-BG and cosmetic-grade petro-BG samples at 2-fold sample dilutions (DF2 samples). The main peak at the center of each of the three chromatograms (retention time (RT): 11.85 min) represents 1,3-BG.

Table 3 shows results of an overall GC-MS purity analysis of the bio-BG and petro-BG samples of FIG. 1. Industrial-grade and cosmetic-grade petro-BG samples were found to have overall higher levels of heavies and lights impurities compared to bio-BG.

TABLE 3
GC-MS purity analysis of 1,3-BG samples

| | | Petro-BG | Petro-BG |
|---|---|---|---|
| | Bio-BG | (cosmetic-grade) | (industrial-grade) |
| Overall Purity [%] | 99.4 | 98.7 | 99.0 |
| BG Heavies [%] | 0.6 | 1.07 | 0.9 |
| BG Lights [%] | 0.0 | 0.17 | 0.1 |

Table 4 shows results of a quantitative analysis of 3-hydroxy-butanal and 4-hydroxy-2-butanone levels in the 1,3-BG samples of FIG. 1. 3-Hydroxy-butanal (RT: 9.51) and 4-hydroxy-2-butanone (RT: 10.08) were detectable as bio-BG specific “lights” compounds that were present at 100-fold higher levels or more in bio-BG samples relative to industrial-grade petro-BG or cosmetic-grade petro-BG samples.

TABLE 4
3-Hydroxy-butanal and 4-hydroxy-2-butanone levels in 1,3-BG
samples as determined by GC-MS

| | | Petro-BG | Petro-BG |
|---|---|---|---|
| Sample ID | Bio-BG | (cosmetic-grade) | (industrial-grade) |
| 3-hydroxy-butanal | 989.8 ppm | Not analyzed | Not analyzed |
| 4-hydroxy-2-butanone | 1212.6 ppm | 8.8 | 6.4 |

An additional bio-BG-specific compound, a heavies compound (compound 9) with a retention time of about 12.5 min, was detected in bio-BG D2 samples and was not detected in petro-BG D2 samples. See FIG. 1. Generally, more numerous heavies compounds were detected in cosmetic-grade petro-BG and industrial-grade petro-BG than in bio-BG. See, e.g., FIG. 1. Heavies compounds detected in both petro-BG and bio-BG samples were found to be present at higher levels in petro-BG samples relative to bio-BG samples, or to be present at lower levels in petro-BG samples relative to bio-BG samples, depending, e.g., on individual heavies compound. See, e.g., FIG. 1. Certain petro-BG specific lights compounds were detected with retention times in the 10.1 min-11.5 min range. Petro-BG DF2 samples of cosmetic-grade and industrial-grade were found to have generally similar numbers and levels of lights and heavies compounds. See, e.g., FIG. 1.

FIG. 2 shows an overlay of an exemplary GC-MS chromatograms of a bio-BG sample and industrial-grade petro-BG and cosmetic-grade petro-BG samples at 20-fold sample dilutions (DF20 samples). The bio-BG specific compounds 3-hydroxy-butanal, 4-hydroxy-2-butanone, and compound 9 were also detected in DF20 1,3-BG samples. Moreover, an additional bio-BG specific heavies compound, compound 7, was detected at a retention time of about 12.05 min. Compound 7 levels were about 1,000 ppm in bio-BG samples. In cosmetic and industrial-grade petro-BG, compound 7 was either not detectable by GC-MS or found to be present at least 100-fold lower concentrations, relative to bio-BG.

FIG. 3 shows an exemplary mass spectrum of bio-BG specific heavies compound 7 observed at a retention time of about 12.05 min in a GC-MS chromatogram, with proposed interpretations of certain mass fragments indicated. Without wishing to be bound by any theory, m/z=161 is believed to be the molecule ion peak of compound 7. Without wishing to be bound by any theory, m/z=183 is believed to be a sodium adduct of the compound 7 molecule ion.

FIG. 4 shows an exemplary mass spectrum of bio-BG specific heavies compound 9 observed at a retention time of about 12.51 min in a GC-MS chromatogram, with proposed interpretations of certain mass fragments indicated. Without wishing to be bound by any theory, m/z=161 is believed to be the molecule ion peak of compound 9. Without wishing to be bound by any theory, m/z=183 is believed to be a sodium adduct of the compound 9 molecule ion.

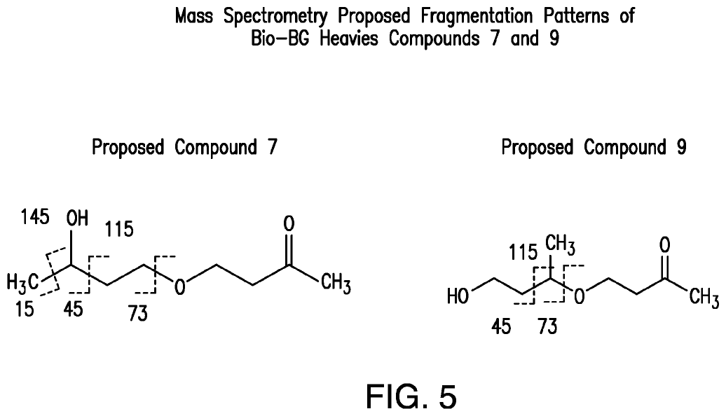

Without wishing to be bound by theory, the fragmentation mass spectra of compounds 7 and 9, e.g., as shown in FIGS. 3 and 4, are believed to suggest that compounds 7 and 9 are or may be structural isomers sharing the same elemental composition (C₈H₁₆O₃). Specifically, compounds 7 and 9 are believed to show similar fragmentation patterns. Individual fragments shared by compounds 7 and 9 were often detectable with different TIC intensities. For example, the mass spectra of compounds 7 and 9 share distinctive 115 m/z and 145 m/z fragments. The 145 m/z fragment of compound 7 was found to have much higher intensity (FIG. 3) than the corresponding 145 m/z fragment of compound 9 (FIG. 4). The 115 m/z fragment of compound 7 was found to have a somewhat lower intensity compared to the corresponding 115 m/z fragment of compound 9. Additional fragments shared in the mass spectra of compounds 7 and 9 include 45 m/z and 73 m/z fragments. The presence of an abundant 145 m/z fragment indicates or is believed to indicate the frequent loss of a methyl group (—CH3) (−15) from compound 7, whereas 73 m/z and 45 m/z fragments indicate or are believed to indicate the presence of hydroxybutyl (73 m/z) and hydroxyl-ethyl (45 m/z) fragments. The prominent 115 m/z fragment of compound 9 indicates or is believed to indicate the frequent loss of a hydroxyl-ethyl moiety from compound 9. Table 5 shows proposed chemical structures for compounds 7 and 9 that were based on the observed mass spectrometry fragmentation patterns, as shown, e.g., in FIG. 3 and FIG. 4. FIG. 5 shows chemical drawings illustrating the proposed structures and proposed fragmentation of compounds 7 and 9 based on proposed mass fragments believed to be observed by mass spectrometry. The proposed structures in FIG. 5 and Table 5 and the proposed fragmentation illustrated in FIG. 5 are not intended to be limiting.

TABLE 5
Proposed chemical structures of compounds 7 and 9

| Compound | Retention | | |
|---|---|---|---|
| ID | Time | Chemical Structure | Compound Name |
| Compound 7 | 12.05 min | 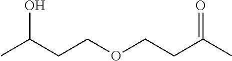 | 4-(3-hydroxybutoxy)butan-2-one (3-hydroxy-butyl-3-oxo-butane ether) |
| Compound 9 | 12.51 min | 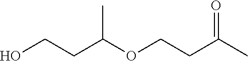 | 4-((4-hydroxybutan-2-yl)oxy)- butan-2-one (2-methyl-3-hydroxy-propyl-3- oxo-butane ether) |

Without wishing to be bound by theory, it is believed that compounds 7 and 9 are or may be products of condensation reactions occurring especially in bio-BG, for example between 3-hydroxy-butanal and 4-hydroxy-2-butanone.

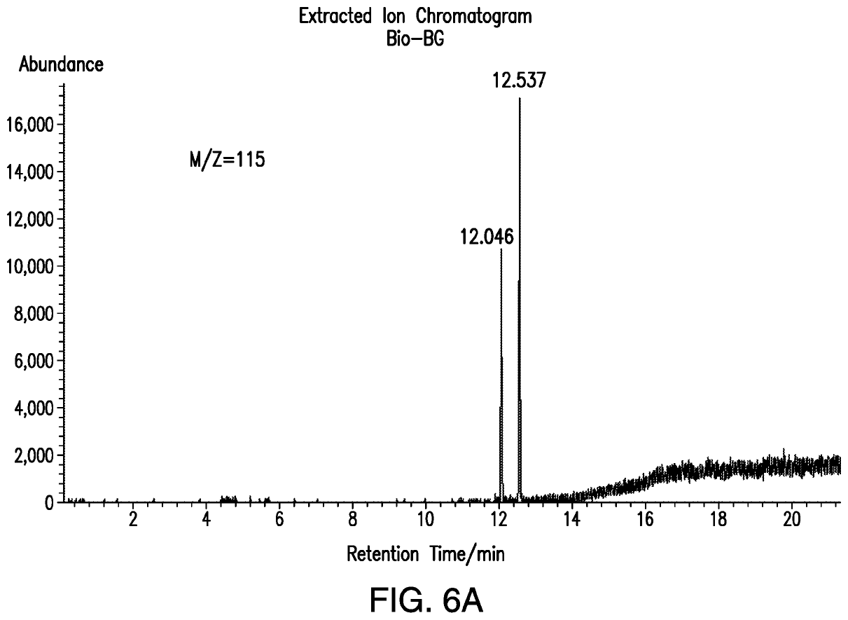

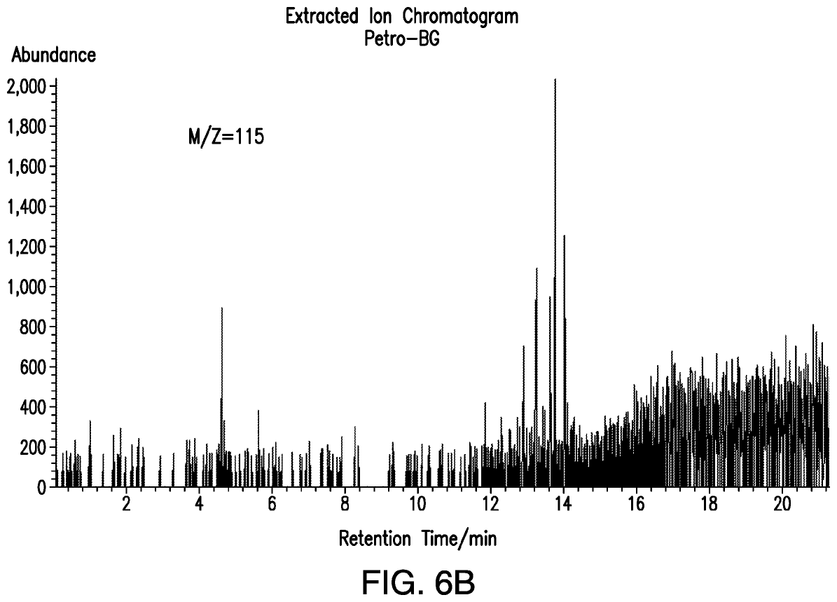

FIG. 6A shows an exemplary extracted ion chromatogram for m/z 115 of a bio-BG sample.

FIG. 6B shows an exemplary extracted ion chromatogram for m/z 115 of a petro-BG sample.

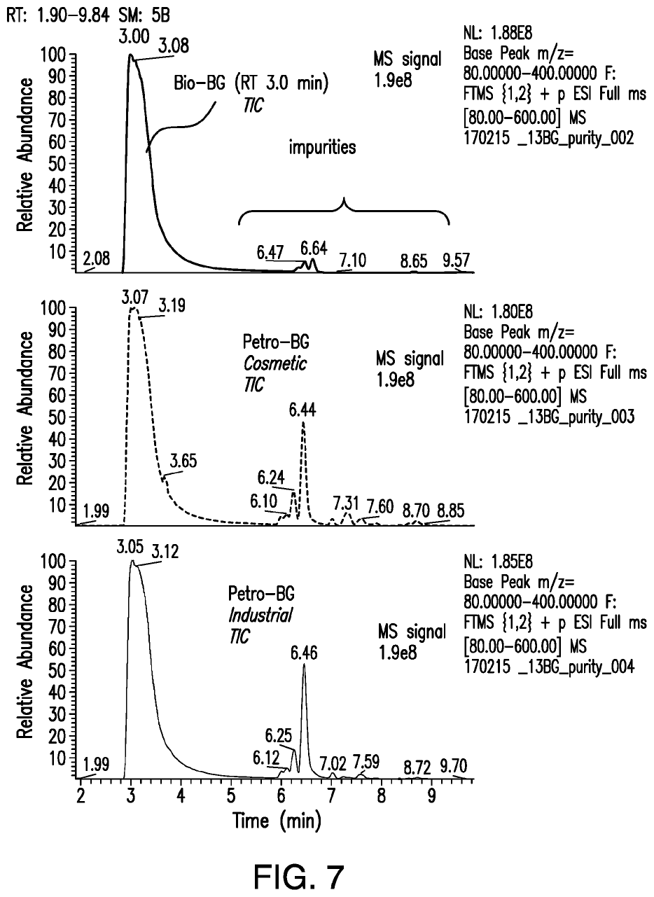

FIG. 7 shows exemplary liquid-chromatography mass spectrometry (LC-MS) chromatograms (TIC: total ion current) of a bio-BG sample (top panel), a cosmetic-grade petro-BG sample (middle panel), and an industrial-grade petro-BG sample (bottom panel). Base peak LCMS chromatograms reveal differences in impurities profiles between bio-BG and petro-BG. The main BG peak elutes early at 3 min retention time followed by the impurities in 5-9 min range. Cosmetic and industrial grades of petro-BG look similar, while bio-BG has lower relative content of impurities. FIGS. 8A-8B compare XIC for the most intense m/z values (peaks eluting at 6.25, 6.45 and 6.65 min) derived from FIG. 7 TIC data.

FIGS. 8A and 8B show results of an LC-MS analysis of exemplary 1,3-BG samples, with proposed interpretations of certain mass fragments indicated in FIG. 8B. The top panel in FIG. 8A shows the total ion current (TIC) profile of a bio-BG sample. The bottom three panels in FIG. 8A illustrate extracted ion current chromatograms (XIC (IEX), +/−10 ppm window around theoretical exact mass of C₈H₁₆O₃ heavies compound) of the bio-BG samples and cosmetic and industrial grade petro-BG samples. Multiple heavies peaks were detected at retention times of 6.2 min, 6.4 min, and 6.6 min in all three samples. See FIG. 8A. Mass spectrometry fragmentation patterns of compounds from the three heavies peaks showed that all three peaks represent molecules with the same elemental composition C₈H₁₆O₃. See FIG. 8B. Without wishing to be bound by theory, the three heavies peaks observed in bio-BG and petro-BG samples are believed to represent structural isomers. The proposed structures in FIG. 8B are not intended to be limiting.

LC-MS analysis further identified a petro-specific heavies compound with a retention time of 7.3 min and an elemental composition of C₈H₁₄O₃ and a molecular weight of 158. See FIG. 9A. Without wishing to be bound by any theory, the observed fragmentation pattern of the petro-BG-specific heavies compound, e.g., as shown in FIG. 9B with proposed interpretations of certain mass fragments indicated, is believed to suggest the chemical structure of 1-4-(4-methyl-1,3-dioxan-2-yl)propan-2-one. See also Table 6. The proposed structures in FIG. 9B and Table 6 are not intended to be limiting.

TABLE 6
Proposed chemical structure of petro-BG-specific compound

| Compound | Retention | | Compound |
|---|---|---|---|
| ID | Time | Chemical Structure | Name |
| Petro- specific heavies compound (C₈H₁₄O₃) | 7.3 min | 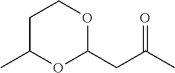 | 1-4-(4-methyl- 1,3-dioxan-2- yl)propan-2-one |

### Example 3

### Identification of Odor Causing Compounds in Bio-BG by GC-MS/O

Bio-BG and petro-BG samples were submitted to Volatile Analysis Corporation (VAC, Grant, AL) to identify off-odor causing compounds using VAC's gas-chromatography mass spectrometry/olfactory (GC-MS/O) analysis service.

VAC's GC-MS/O service involves a trained odor judge evaluating GC effluent and rating its odor intensity and character, e.g., by providing qualitative odor descriptors. This sensory information, along with the odor's GC retention time (RT), is recorded and computationally aligned with total ion chromatogram MS peaks. By understanding what chemical peaks exhibits off-odor related to the odor problem, any offensive chemical off-odor can be identified and measured. Industrial-grade and cosmetic-grade petro-BG is commercially available from several vendors. Solid Phase Microextraction (SPME) was used for sample preparation. SMPE is a solid phase extraction sampling technique that involves the use of a fiber coated with a liquid or solid extracting phase that can extract both volatile and non-volatile analytes from liquid samples or a gas phase.

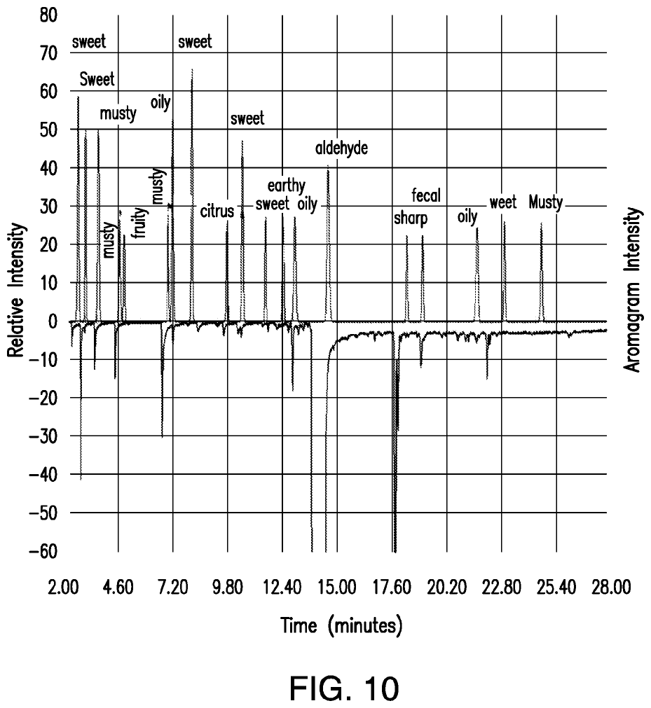

FIG. 10 and FIG. 11 show exemplary GC-MS/O analysis results for cosmetic-grade petro-1,3-BG (FIG. 10) and bio-BG (FIG. 11). The upper traces and upward pointing peaks in FIG. 10 and FIG. 11 represent the human sensory score of odor intensity obtained by olfactory analysis by a trained VAC odor judge. The lower traces and downward pointing peaks in FIG. 10 and FIG. 11 represent GC-MS chromatogram peaks (TIC), with the largest peaks at about 13 min retention time representing 1,3-BG.

GC-MS/O analysis results, e.g., as illustrated in FIGS. 10 and 11 showed a larger overall number of odorous fractions in bio-BG than cosmetic-grade petro-BG, especially at retention times shorter than the retention time of 1,3-BG. At retention times longer than the retention time of 1,3-BG slightly fewer odorous compounds were detected in bio-BG than in cosmetic-grade petro-BG. Many of the odor causing fractions in bio-BG and cosmetic-grade petro-BG did not include compounds showing strong, or any, UV absorbance. Cosmetic grade petro-BG included GC fractions with sweet (5 fractions), musty (4 fractions), fruity (1 fraction), oily (3 fractions), citrus (1 fraction), earthy (1 fraction), aldehyde (1 fraction), sharp (1 fraction), or fecal (1 fraction) odors. Bioderived 1,3-BG included GC fraction with sweet (6 fractions), musty (6 fractions), oily (4 fractions), aldehyde (1 fraction), sharp (2 fractions), buttery (1 fraction), solvent (1 fraction) or unknown (1 fraction) odors. The bioderived 1,3-BG did not include fractions with fecal, earthy, or citrus odors. The bioderived 1,3-BG included fractions with buttery or solvent odors that were not present in cosmetic-grade petro-BG. The bioderived 1,3-BG did not include fractions with fecal, musty, or sharp odors and GC retention times longer than 1,3-BG.

Overall, bio-BG was characterized by GC-MS/O analysis as having predominantly “oily, paint-like, glue-like” odor, while the petro-BG was characterized as “sharp, sweet, alcoholic, and fruity.” In particular, GC-MS/O analysis identified 8 unique odor notes of 4 known compounds (methyl vinyl ketone (MVK), 4-methyl-1-penten-3-one, 1-hepten-3-one, and diacetyl) and of 4 unknown compounds.

### Example 4

### Identification of Odor Causing Compounds in Bio-BG by GC-MS

GC-MS analyses of liquid samples of bio-BG and of headspace samples from bio-BG (by SPME-GCMS) resulted in the proposed identification of several odor causing impurities, which are listed in Table 7. Some of the identified compounds (e.g., 1-hydroxy-2-propanone, 1,2-propanediol, 1,3-propanediol, 2,3-butanediol, 3-hydroxy-2-butanone) were only identified by liquid GC-MS analysis, which may indicate low volatility of the identified compounds. It is commonly believed that low volatility compounds are less likely than higher volatility compounds to contribute substantially to any off-odor of a liquid sample, such as a liquid bioderived 1,3-BG sample. Other compounds, such as acetaldehyde, 3-buten-2-one or methyl vinyl ketone, diacetyl, crotonaldehyde, were only detected in the headspace of a liquid bioderived 1,3-BG sample, indicating that these compounds are present in the liquid fraction of bioderived 1,3-BG samples only at concentrations below the liquid GC-MS detection limit. Compounds found only in headspace are likely to contribute to the off-odor of bioderived 1,3-BG.

TABLE 7
Summary of proposed odor causing impurities identified in an exemplary bioderived 1,3-BG product

| | | | Boiling | | |
|---|---|---|---|---|---|
| Compound | Structure | MW | Pt (° C.) | Odor Notes | Other Comments |
| Acetaldehyde | 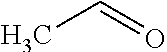 | 44.05 | 21 | Sharp, sweet, pungent | Only detected in the headspace Possibly formed during GC-MS injection |
| 4-Hydroxy-2- butanone | 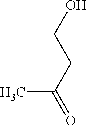 | 88.11 | 73 | Fruity, musty, sweet | Possible dehydration to MVK Detected in liquid and headspace |
| 3-Buten-2-one (methyl vinyl ketone, MVK) | 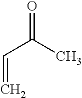 | 70.09 | 81 | Pungent | Possible dehydration product of 4-hydroxy-2- butanone at high temperature Only detected in the headspace |
| Diacetyl | 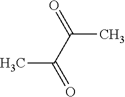 | 86.09 | 87 | Buttery, sweet | Only detected in the headspace |
| 2-Butenal (Crotonaldehyde) | 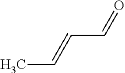 | 70.09 | 104 | Pungent, suffocating | Possible dehydration product of 3-OH-butanal at high temperature Only detected in the headspace |
| 1-Hydroxy-2- propanone | 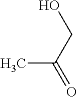 | 74.08 | 145 | Pungent, sweet, carmellic ethereal | Detected in the liquid, not headspace |
| 3-Hydroxy-2- butanone (acetoin) | 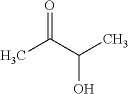 | 88.11 | 148 | Fatty, wet, buttery | Detected in the liquid, not headspace |
| 3-Hydroxy-butanal (3-Hydroxy- butyraldehyde) | 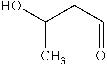 | 88.1 | 162 | | Possible dehydration to crotonaldehyde at high temperature Detected in liquid and headspace |
| 2,3-Butanediol | 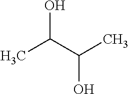 | 90.07 | 183 | Green, buttery | Detected in the liquid, not headspace |
| 1,2-Propanediol | 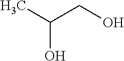 | 76.09 | 186 | Sweet, ether-like | Detected in the liquid, not headspace |
| 1,3-Propanediol | 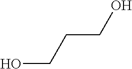 | 76.09 | 214 | Odorless? | Detected in the liquid, not headspace |

### Example 5

### Degradation of 1,3-BG Using Heat and Formation of Dehydration Products

During the development of the GC-MS method described in Example 4, it was found or believed that methyl vinyl ketone (MVK, 3-butene-2-one) and crotonaldehyde (Cr-Ald) were formed during injection inside a GC inlet port at temperatures of 250° C. and as low as 150° C. MVK was formed or believed to be formed through dehydration of 4-hydroxy-2-butanone and Cr-Ald was formed or believed to be formed through dehydration of 3-hydroxy-butanal, as shown in the proposed schematic illustrated in FIG. 5. MVK and Cr-Ald are odor causing compounds with reported odor thresholds of 200 ppb (MVK) and 35 to 120 ppb (Cr-Ald). The low odor thresholds of MVK and Cr-Ald mean that odor causing MVK and Cr-Ald cause noticeable odor at levels that are lower than the detection limits of analytical methods such as GC-MS. Cr-Ald has a reported odor threshold of 35 to 120 ppb and MVK has a reported odor threshold of 200 ppb.

Prompted by the observation of MVK and Cr-Ald formation in the course of GC-MS analytics, it was tested whether the same proposed dehydration of 4-hydroxy-2-butanone and 3-hydroxy-butanal also occurred in a batch distillation reboiler, where temperatures of 120-130° C. are commonly observed and where residence times can exceed 6 hours. Three 2 mL test samples were prepared in 20 mL GC-MS headspace vials as follows:
1) Cosmetic-grade petro-BG
2) Cosmetic petro-BG spiked with 100 ppm of 3-hydroxy-butanal
3) Cosmetic petro-BG spiked with 100 ppm of 4-hydroxy-2-butanone

Test samples 1)-3) were then heated to 120° C. in a silicone oil bath, incubated in the oil bath for 6 hours, and analyzed by SPME-GCMS and GCMS. The test results are shown in Table 8 and Table 9.

TABLE 8
SPME-GCMS purity results (TIC peak areas) of neat and spiked cosmetic-grade-
petro-BG before and after heating at 120° C. for 6 hours

| | Cosmetic-grade | | 3-Hydroxy-Butanal | | 4-Hydroxy-2-Butanone | |
|---|---|---|---|---|---|---|
| | petro-BG | | (100 ppm) | | (100 ppm) | |
| Chemical | Feed | Product | Feed | Product | Feed | Product |
| 4-Hydroxy-2-Butanone | 332,496 | 544,781 | 182,234 | 428,011 | 3,694,645 | 1,039,023 |
| MVK | 236 | 594,247 | 0 | 618,230 | 1,146,053 | 7,022,102 |
| Cr-Ald | 0 | 3,210,177 | 8,236,678 | 12,466,838 | 0 | 3,126,029 |
| 3-Hydroxy- | 50,833 | 16,297 | 41,336 | 26,414 | 28,168 | 22,475 |
| Butanal | | | | | | |

TABLE 9
GC-MS purity results (TIC peak areas) of neat and spiked cosmetic-grade petro-
BG before and after heating at 120° C. for 6 hours

| | | [3-OH- | [4-OH-2- | | | |
|---|---|---|---|---|---|---|
| | | Butanal] | Butanone] | [1,3 BG (BDO) | [1,3-BG (BDO) | |
| Sample | | ppm | Ppm | Heavies] % | Lights] % | [Purity] % |
| Feed | Cosmetic-grade | 8.81 | 16.03 | 0.71 | 0.11 | 99.2 |
| Product | petro-BG | 13.86 | 19.3 | 0.7 | 0.1 | 99.2 |
| Feed | 3-OH-butanal | 99.4 | 16.23 | 0.69 | 0.1 | 99.2 |
| Product | (100 ppm) | 12.22 | 18.42 | 0.64 | 0.09 | 99.3 |
| Feed | 4-OH-2-butanone | 15.62 | 268.18 | 0.66 | 0.09 | 99.2 |
| Product | (100 ppm) | 19.97 | 69.23 | 0.81 | 0.08 | 99.1 |

Table 8 shows that higher levels of Cr-Ald were found in 3-hydroxy-butanal-spiked samples than in neat cosmetic-grade petro-BG or in 4-hydroxy-2-butanone-spiked samples. Furthermore, higher levels of MVK were found in 4-hydroxy-2-butanone-spiked samples than in neat cosmetic-grade petro-BG or in 3-hydroxy-butanal-spiked samples. Cr-Ald and MVK levels increased following heating of samples at 120° C. for 6 hours.

Table 9 shows that 3-hydroxy-butanal and 4-hydroxy-2-butanone levels are decreased in 3-hydroxy-butanal and 4-hydroxy-2-butanone-spiked petro-BG samples following heating of the samples at 120° C. for 6 hours. Overall purity levels of neat and 3-hydroxy-butanal and 4-hydroxy-2-butanone-spiked petro-BG samples were found to be essentially unchanged by the heat treatment.

This experiment confirms that 4-hydroxy-2-butanone can degrade to MVK and 3-hydroxy-butanal can degrade to Cr-Ald, two potent odor byproducts, under the conditions of a batch distillation process.

### Example 6

### Activated Carbon Treatments

Activated carbon is commonly used in laboratory scale and industrial scale production and purification processes to remove color- and odor-causing impurities from a product, such as petro-BG. For example, U.S. Pat. No. 8,445,733 B1, the entire contents of which are incorporated by reference herein, purports to describe methods for reducing odor of a petro-BG product using certain activated carbon preparations.

This example presents results of experiments in which bio-BG products were treated with activated carbon preparations.

### Description of Activated Carbons Tested

Types of activated carbon tested and their properties:
Cabot Darco S-51A M-1967 (Darco; Cabot Corp., Boston, MA). This activated carbon preparation is coal-based, steam activated, neutralized with pH 6-8, and presented in a pulverized form. It is frequently used to remove, taste, odor, or light color in sugar applications.
Calgon FILTRASORB 300 (FS 300; Calgon Carbon Corp., Moon Township, PA). This activated carbon preparation is coal-based and presented in a 12×40 granular form. It is frequently used to remove taste, odor, and color from water, wastewater, and industrial and food processing streams.
Calgon BG HHM (BG HHM; Calgon Carbon Corp., Moon Township, PA). This activated carbon preparation is wood-based, acid-activated, and presented in a pulverized form. It is designed by the manufacturer for decolorization in food and beverage processes and pharmaceutical product purification. Specifically, this preparation was developed to effectively adsorb high and low molecular weight organic impurities and meets the Food Chemical Codex requirements.
Coconut shell (CS; Calgon Carbon Corp., Moon Township, PA). This activated carbon preparation is coconut shell-based and presented in a granular form. The preparation features very large internal surface areas characterized by micro-porosity along with relatively high hardness and low dust. It is frequently used for water and critical air purification applications such as in point of use water filters and respirators.
Calgon CPG-LF (CPG-LF; Calgon Carbon Corp., Moon Township, PA). This activated carbon preparation is coal-based, acid-washed with neutral pH, presented in a 12×40 granular form, and contains reduced iron and ash levels. The preparation has a strongly adsorbing pore structure that is designed for the adsorption of organics, color bodies, and odor molecules.

### Activated Carbon Testing by Shake Flask Method

A shake flask method was used to quickly test multiple activated carbon preparations with minimal or low 1,3-BG material requirement. The test procedure was as follows:
1) The carbon sample was pulverized using a mortar and pestle;
2) the carbon was then washed multiple times with water;
3) the carbon was allowed to completely dry, e.g., using an oven;
4) equal amounts of each carbon preparation and bio-BG were loaded in a 125 mL flask with a target ratio of 0.2 g carbon/g bio-BG;
5) the flasks were shaken at 40° C. and 200 rpm for 24 hours;
6) the carbon was separated from the bio-BG using a 0.22 μM vacuum filter;
7) the bio-BG was analyzed for odor, purity, and UV.

In one shake flask experiment, three activated carbon preparations were tested: FS 300, CS, and BG HHM. Table 10 shows GC-MS purity data for the bio-BG containing feed, which was not treated with an activated carbon preparation, and three bio-BG samples, which were treated with different activated carbon preparations.

TABLE 10
GC-MS purity results for untreated bio-BG feed and bio-BG samples
treated with indicated activated carbon preparations.

| | Bio-BG | | | |
|---|---|---|---|---|
| Sample ID | Feed | FS 300 | CS | BG HHM |
| 1-Hydroxy-2-Propanone [ppm] | 9.8 | 0.6 | 2.0 | 1.0 |
| 1,2-Propanediol [ppm] | 4,324 | 3,889 | 4,161 | 4,252 |
| 1,3-Propanediol [ppm] | 272 | 191 | 197 | 221 |
| 2,3-Butanediol [ppm] | 5,591 | 4,787 | 5,103 | 5,196 |
| 3-Hydroxy-2-butanone [ppm] | 6.6 | 5.4 | 6.3 | 5.8 |
| 4-Hydroxy-2-butanone [ppm] | 1,636 | 1,104 | 1,172 | 1,200 |
| 1,3-BG Heavies [%] | 1.5 | 0.9 | 0.9 | 1.1 |
| 1,3-BG Lights [%] | 0.6 | 0.5 | 0.5 | 0.5 |
| Purity [%] | 97.9 | 98.6 | 98.6 | 98.4 |
| 3-Hydroxy-Butanal- | 302,881 | 217,424 | 167,523 | 199,657 |
| [peak area DF2] | | | | |

The FS 300 treated bio-BG sample showed the biggest reduction in 4-hydroxy-2-butanone. The CS treated bio-BG sample showed the biggest reduction in 3-hydroxy-butanal. Treatments with all tested activated carbon preparations reduced 4-hydroxy-2-butanone and 3-hydroxy-butanal in bio-BG samples. FS 300 and CS increased the purity of bio-BG by 0.7% and BG HHM increased the purity of bioderived 1,3-BG by 0.5%.

A second shake flask study compared CPG-LF activated carbon against FS 300 and Darco activated carbon preparations. 3-Hydroxy-butanal was quantified by SPME-GCMS. The bio-BG feed sample tested in the second study was obtained from a final bio-BG distillate (see Example 1), whereas the bio-BG feed sample tested in the first study was obtained from an earlier distillation fraction and differed in its overall purity level. SPME and GC-MS purity results are shown in Table 11 and Table 12.

TABLE 11
SPME purity results (peak areas for identified (proposed) compounds)

| | Petro- | | | | |
|---|---|---|---|---|---|
| | BG* | | | | |
| | (cosmetic | Bio-BG | | | |
| Identified Peak | grade) | Feed | FS 300 | Darco | CPG-LF |
| 4-Hydroxy-2-Butanone | 562,994 | 5,626,300 | 4,506,134 | 2,753,086 | 3,718,230 |
| MVK | 17,635 | 388,829 | 139,931 | 56,659 | 171,838 |
| 3-Hydroxy-Butanal | 44,693 | 102,148 | 79,752 | 62,263 | 66,764 |
| Cr-Aid | 42,506 | 326,670 | 197,069 | 457,111 | 290,244 |
| Acetol | 60,732 | 75,908 | 10,218 | 3,823 | 18,760 |
| Pentane, 2,4-epoxy-, trans- | 0 | 2,642 | 2,444 | 1,849 | 1,479 |
| Biacetyl | 15 | 112,048 | 23,916 | 829 | 87 |
| Acetone | 4,515 | 263,342 | 67,926 | 13,242 | 67,703 |
| Dodecane | 31,170 | 21,327 | 18,967 | 19,443 | 14,314 |
| 1,3-Butanediol, diacetate | 512,156 | 40,284 | 37,294 | 15,608 | 31,271 |

*commercially available

TABLE 12
GCMS purity results

| | [bio-BG | [bio-BG | | [3-OH- | [4-OH-2- | | | |
|---|---|---|---|---|---|---|---|---|
| Sample | Heavies] | Lights] | [Purity] | Butanal] | Butanone] | [IPA] | [n-But] | [12PDO] |
| ID | % | % | % | ppm | ppm | ppm | ppm | ppm |
| Feed | 0.22 | 0.02 | 99.8 | 523 | 359 | — | — | 39 |
| FS 300 | 0.19 | 0.02 | 99.8 | 319 | 253 | — | — | 39 |
| Darco | 0.18 | 0.01 | 99.8 | 239 | 142 | 3.68 | — | 40 |
| CPG-LF | 0.17 | 0.01 | 99.8 | 209 | 218 | — | — | 40 |

All three activated carbon preparations were found to reduce 3-hydroxy-butanal and 4-hydroxy-2-butanone and removed some unknown heavies and lights.

The bio-BG feed and FS 300 and CPG-LF treated bio-BG samples were analyzed by a trained odor panel. The odor panel results showed that carbon treatment did not make it more difficult to distinguish the bio-BG samples from a commercially available cosmetic-grade petro-BG material. Qualitatively, the odor intensity of the activated carbon-treated bio-BG material was slightly lower than the feed material.

Activated Carbon Results from 0.59″ Column Runs

FS 300 was tested in a column format for its ability to remove impurities and odor from bio-BG.

A first FS 300 column run was performed using a high purity bio-BG “lights” distillate. See Example 1 and Table 13. To avoid or reduce the addition of water, the FS300 material was loaded dry on a 0.59″ column. Operation parameters for the FS 300 column run are shown in Table 13.

TABLE 13
Pilot column operational parameters for activated carbon experiments.

| Parameter | Value | Units |
|---|---|---|
| Flow rate | 3 | ml/min |
| Resin height | 42 | in |
| Col diameter | 0.59 | in |
| Col area | 0.0019 | ft\hat{ }2 |
| Bed volume | 188.2 | mL |
| Flux | 0.42 | GPM/ft\hat{ }2 |
| Contact time | 62.7 | min |
| Temperature | 80 | degF |

Table 14 shows the results of an analysis of FS 300-treated (feed) and untreated (product) bio-BG samples. UV absorbance at 270 nm of the FS 300 treated bio-BG sample was reduced by 10-fold and the overall purity of the bio-BG product increased by 0.1%.

TABLE 14
Bio-BG GC-MS analysis of feed and product of a FS 300 column run

| | Feed | Product |
|---|---|---|
| UV at 270 nm | 0.4423 | 0.040 |
| Water | 0.1711% | 0.3432% |
| 4-Hydroxy-2-butanone | 0.0124% | 0.0068% |
| 2,3-Butanediol | 0.0017% | 0.0028% |
| 1,2-Propanediol | 0.0172% | 0.0115% |
| 1,3-Propanediol | 0.0096% | 0.0000% |
| Lights | 0.0300% | 0.0199% |
| Heavies | 0.2996% | 0.2988% |
| Purity | 99.60% | 99.70% |

50 mL bio-BG fractions were collected throughout the FS 300 column run and each fraction's odor was screened directly from the 50 mL tubes by an untrained panel. Based on this initial screen, select FS 300 fractions of bio-BG were pooled and submitted to VAC. Odor analysis by trained odor judges indicated that the FS300 treatment did not reduce the odor of the tested bio-BG samples.

A second activated carbon column run was performed using CPG-LF (12×40 granular size in a 0.59″ diameter column) and a bio-BG heavies distillate that was less pure and had more intense odor than the bio-BG lights distillate. The CPG-LF column was loaded wet to prevent channeling and improve absorption of bio-BG impurities to the CPG-LF activated carbon. Six bio-BG fractions were collected from the CPG-LF column. The overall purity of the CPG-LF column fractions by 0.7% and reduced the UV absorbance of the CPG-LF fractions by 10-fold relative to the bio-BG feed. No improvement in the relative odor intensity was observed for any of the six CPG-LF column fractions relative to the bio-BG feed loaded onto the column.

In conclusion, it is believed that this example illustrates that activated carbon treatments of bio-BG samples were not found to result in a substantial reduction of odor in bio-BG. This observation differs from the odor-reducing effects of activated carbon on petro-BG described in the art, e.g., in U.S. Pat. No. 8,445,733.

### Example 7

### Base Addition to Final Distillation Reboiler

Base addition to crude or low-quality petro-BG has been reported to aid in the reduction of odor of petro-BG preparations. See, e.g., JP-A-7-258129, U.S. Pat. No. 6,376,725, and EP 1046628, the entire contents of each of which are incorporated by reference herein. This example describes results of experiments using base addition to reduce odor of bio-BG.

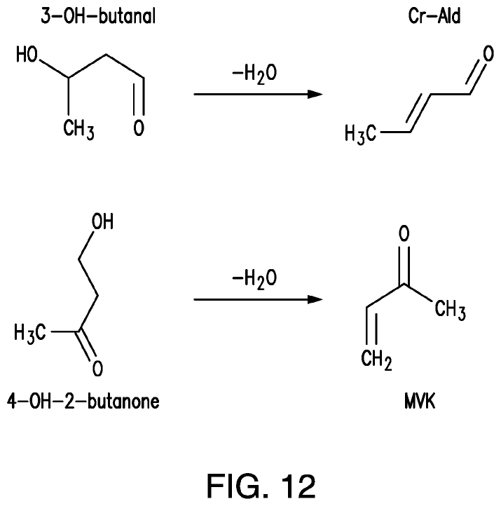

Without wishing to be limited by theory, it is believed that base addition to bio-BG may reduce dehydration of 3-hydroxy-butanal to crotonaldeyde and of 4-hydroxy-2-butanone to methyl-vinyl-ketone (see, e.g., Example 5 and FIG. 12), and to promote the reaction of aldehydes and ketones to heavier, less volatile, compounds. It is believed that in the presence of a base, aldehydes and ketones, such as 4-hydroxy-2-butanone and 3-hydroxy-butanal, can form enolates and undergo condensation reactions yielding certain enols and aldols. Enols and aldols can oligomerize further to produce heavier boiling compounds that can be separated from bio-BG by distillation.

In the examples described below, base was added to a crude bio-BG preparation obtained after heavies distillation in a lab-scale (2 L) batch distillation system as described, e.g., in Example 1. 99.8% pure bio-BG having an intense odor was used as a “feed” for a distillation system. 2.73 mL of 10 M sodium hydroxide (NaOH) was added to the reboiler (equal to 0.2 wt % NaOH). Distillation was done at a low pressure of 10-11 Torr and low reboiler temperatures between 118 and 124° C. UV absorbance analysis of the sample showed relatively high UV absorbance. GC-MS analysis results of an exemplary bio-BG distillation run with added base are described in Table 15.

TABLE 15
Bio-BG products of distillation process in presence of added base

| | | [1,3- | | | | | | | | | | |
|---|---|---|---|---|---|---|---|---|---|---|---|---|
| | [1,3-BG | BG | | | | | | | [cis- | [trans- | | |
| | (BDO) | (BDO) | | [3-OH- | [4-OH-2- | | | [3-buten- | Crotyl | Crotyl | Residence | |
| | Heavies] | Lights] | [Purity] | Butanal] | butanone] | [IPA] | [n-But] | 2-ol] | alcohol] | alcohol] | Time | |
| Description | % | % | % | ppm | ppm | ppm | ppm | ppm | ppm | ppm | min | Odor Notes |
| Feed | 0.2 | 0.05 | 99.8 | 132.3 | 137.2 | — | — | — | — | — | — | intense |
| Cut #1 | 0.78 | 0.08 | 99.1 | 5.1 | 21.5 | — | — | — | — | — | 188 | slight |
| Cut #2 | 0.49 | 0.04 | 99.5 | 52.8 | 14.4 | — | — | — | — | — | 248 | slight |
| Cut #3 | 0.2 | 0.01 | 99.8 | 13.2 | 14.9 | — | — | — | — | — | 281 | slight |
| Cut #4 | 0.11 | 0.02 | 99.9 | 4.1 | 13.6 | — | — | — | — | — | 317 | slight |
| Cut #5 | 0.06 | | 99.9 | 2.0 | 9.7 | 84 | — | — | — | — | 371 | noticeable |
| Cut #6 | 0.06 | 0.09 | 99.9 | 4.4 | 12.4 | 2,649.1 | 68.5 | 249.8 | 21.1 | 114.3 | 410 | very |
| | | | | | | | | | | | | noticeable |
| Cut #7 | 0.2 | 0.71 | 99.1 | 5.3 | 9.0 | 6,251.5 | 218.4 | 604.1 | 72.0 | 338.8 | 445 | intense |
| Cut #8 | 0.26 | 0.77 | 99 | — | 12.8 | 4,553.6 | 200.1 | 528.7 | 63.4 | 315.4 | 516 | intense |

Several high-purity bio-BG distillation fractions having a higher purity and reduced odor relative to the feed were obtained, such as cut #4 of Tables 15 and 16. NaOH remained in the reboiler as distillate was removed resulting in an increased concentration of base in the reboiler over time. Without wishing to be bound by theory, it is believed that this increase in base concentration combined with long bio-BG residence times resulted in formation of isopropyl alcohol (IPA), n-butanol (n-But), cis- and trans-crotonyl alcohol, and 3-buten-2-ol, all of which have an intense odor. Cut #4 was the cleanest bio-BG fraction produced, as determined by GC-MS. See Tables 15 and 16. Cut #4 also had the lowest level of MVK and Cr-Ald found in a distillation fraction, as analyzed by SPME-GCMS. See Tables 15 and 16. The overall lowest levels of MVK and Cr-Ald, however, were found in the bio-BG feed. The feed odor is believed to be due to the presence of certain bio-BG lights components. The odor of cuts #3 and #4 was reduced relative to the bio-BG feed, as determined by an odor panel. See Tables 15 and 16. Nonetheless, cuts #3 and #4 were found by the same odor panel to have a higher odor intensity (and different odor characteristics) than commercially available cosmetic-grade petro-BG.

TABLE 16
SPME-GCMS analysis of the feed and select
cuts of base addition to final distillation. The numbers
are peak areas and are comparative, not quantitative.

| Identified | | | | | |
|---|---|---|---|---|---|
| (Proposed) | | | | | |
| Peak | Feed | Cut #3 | Cut #4 | Cut #5 | Cut #6 |
| 4OH-2- | 1,759,686 | 280,196 | 264,524 | 243,359 | 208,485 |
| Butanone | | | | | |
| MVK | 1,580,097 | 3,360,623 | 3,013,159 | 5,291,548 | 6,011,029 |
| 3-hydroxy- | 44,709 | 18,087 | 17,946 | 15,157 | 12,154 |
| Butanal | | | | | |
| Croton- | 788,971 | 3,105,566 | 2,332,291 | 2,970,362 | 7,133,167 |
| aldehyde | | | | | |
| Biacetyl | 223,711 | 4,252 | 2,205 | 166,213 | 865,409 |
| Acetone | 981,759 | 56,394 | 72,690 | 886,831 | 2,294,491 |
| Dodecane | 23,864 | 8,556 | 6,448 | 5,596 | 5,880 |
| 1,3-Bu- | 7,093 | 72,340 | 37,811 | 27,897 | 25,455 |
| tanediol, | | | | | |
| diacetate | | | | | |
| Acetalde- | 24,861 | 10,919 | 16,261 | 35,741 | 187,705 |
| hyde | | | | | |

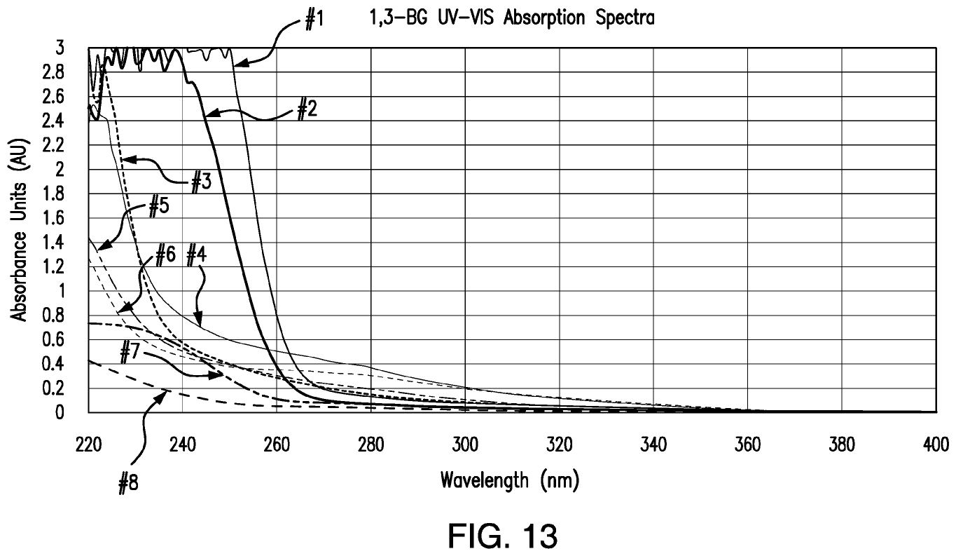

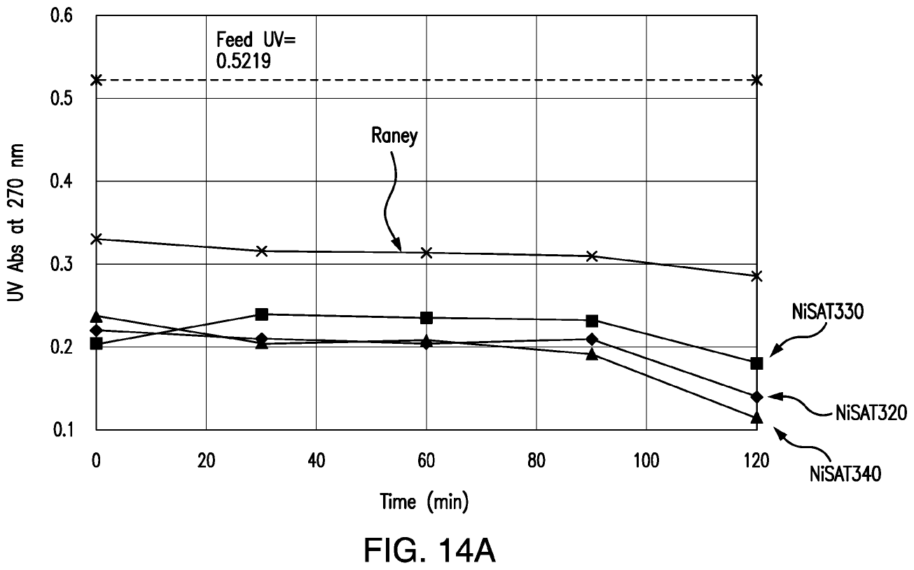

FIG. 13 shows overlaid UV-VIS spectra of several 1,3-BG preparations. Cut #4 (preparation #7 in FIG. 13) has the lowest but still relatively high absorbance of all the materials except for a sodium borohydride treated version of cut #4 (preparation #8 in FIG. 13, see Example 9). Several commercially available petro-BG preparations (e.g., preparations #3 and #4 in FIG. 13 (cosmetic grade) and preparations #5 and #6 in FIG. 13 (industrial grade)) showed higher UV-VIS absorbance than Cut #4 (preparations #7 and #8 in FIG. 13). UV absorbance was not found to correlate to the odor intensities or character of the tested 1,3-BG preparations.

In conclusion, it is believed that this example illustrates that base additions to the final distillation reboiler reduced UV-VIS absorbance of bio-BG preparations, and did not noticeably improve the odor characteristics of bio-BG preparations. The latter observation differs from the odor-reducing effects of base additions described in the literature in connection with petro-BG purification processes. See, e.g., JP-A-7-258129, the entire contents of which are incorporate by reference herein.

### Example 8

### Hydrogenation

Hydrogenation has been reported to aid in the production of high-purity petro-BG and in the reduction of levels of odor-causing aldehydes in petro-BG preparations. This example describes results of experiments using hydrogenation to reduce odor of bio-BG.

Preliminary experiments are believed to have demonstrated that extended hydrogenation (>3-4 hours) of petro-BG with a Raney Nickel catalyst resulted in IPA and butanol formation and increased UV absorbance at 270 nm. This observation is believed to have demonstrated that any IPA and butanol formation observed following nickel-catalyzed hydrogenation of bio-BG may not result from specific trace impurities originating from the bio-BG fermentation processes.

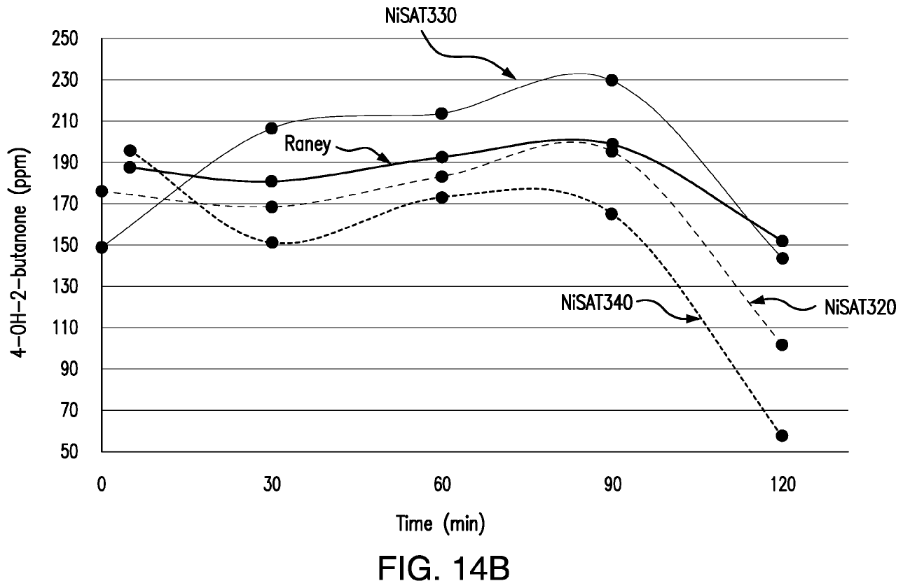

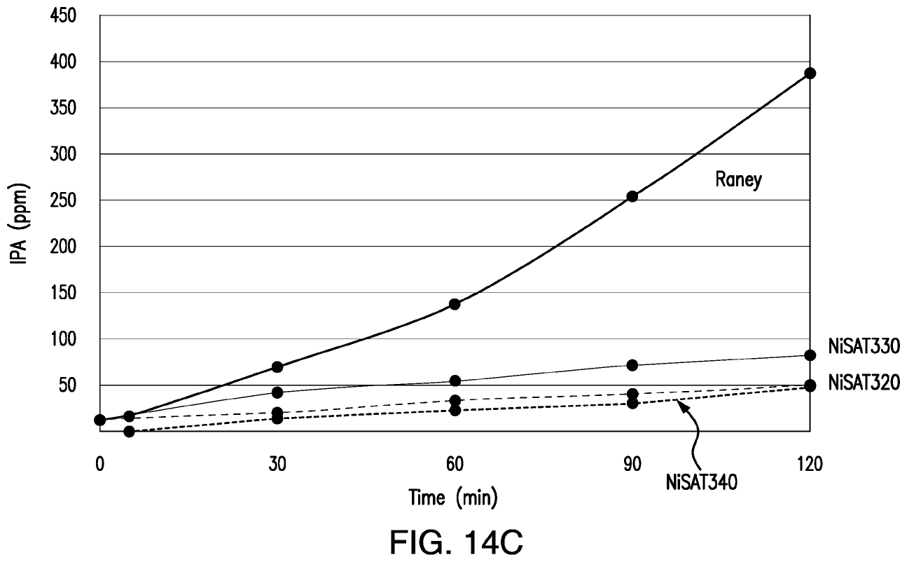

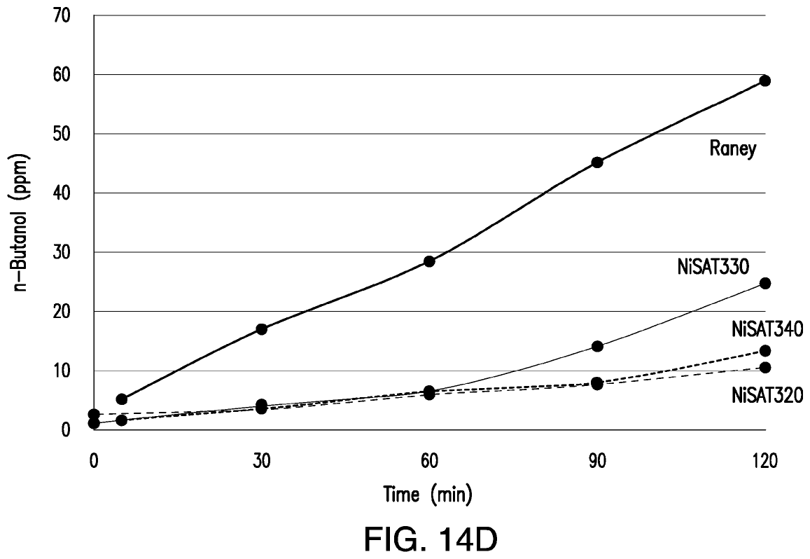

Three nickel catalysts were tested in bio-BG hydrogenation reactions: NiSAT320R, NiSAT330R, and NiSAT340R (Clariant, Muttenz, Switzerland). It was found that reduced residence time with nickel catalysts improved the purity of bio-BG preparations and reduced by-product formation. Three NiSAT catalysts were tested at 1% weight loading and performance was compared to a Raney Nickel catalyst. Operating conditions were 130° C., 500 psi, and roughly 2-hour reaction time. In FIGS. 14A and 14B, and FIGS. 14C and 14D, zero minutes refers to the time at which the hydrogenation reactor reached the target temperature of 130° C. The heating-up period was between 16-20 minutes. The end point of 120 minutes refers to the combined residence time at the target temperature of 130° C. and a cool down period between 15-20 minutes.

FIGS. 14A, 14B, 14C, and 14D show results of bio-BG hydrogenation reactions. A reduction of UV absorbance and 4-hydroxy-butanone levels was observed after extended hydrogenation times of >90 minutes. See FIG. 14A and FIG. 14B. Increased IPA and n-butanol levels were found (proposed) in bio-BG already after hydrogenation times as short as 30 minutes, and further increases were observed over time. See FIG. 14C and FIG. 14D. Raney nickel was found to increase IPA and n-butanol levels more strongly than NiSAT320®, NiSAT330®, or NiSAT340® catalysts.

In conclusion, this example illustrates that extended hydrogenation of bio-BG may reduce UV absorbance and the levels of certain contaminants, such as 4-hydroxy-butanone, while elevating levels of other compounds, such as IPA or n-butanol. These results suggest that hydrogenation may affect the purity and odor-characteristics of bio-BG differently than the purity and odor-characteristics of petro-BG, as described in literature related to petro-BG isolation.

### Example 9

### Sodium Borohydride (NaBH₄)

This example describes results of experiments using sodium borohydride (NaBH₄) to reduce odor of bio-BG, e.g., through elimination of impurities such as MVK or Cr-Ald.

A 20 g sample of bio-BG was reduced with 1000 ppm equivalents (20 mg) of NaBH₄. Feed and product samples were submitted for SPME-GCMS and GCMS analyses and qualitatively assessed for their odor characteristics. SPME-GCMS and GCMS analysis results are shown in Table 17 and Table 18.

TABLE 17
SPME-GCMS analysis of NaBH₄-treated bio-BG (peak areas)
(proposed compounds).

| Identified peak | Feed | Product |
|---|---|---|
| 4OH-2-Butanone | 5,222,205 | 496,525 |
| MVK | 507,607 | 13 |
| 3-OH-Butanal | 69,187 | 62,038 |
| Crotonaldehyde | 268,673 | 148 |
| Acetone | 125,189 | 33,449 |
| Acetaldehyde | 9,011 | 824 |
| Biacetyl | 79,223 | 202 |
| Paraldehyde | 330 | 113 |
| Dodecane | 159 | 200 |
| Acetol | 233,152 | 84 |
| 1,3-Butanediol, diacetate | 28,443 | 148 |

TABLE 18
GCMS liquid analysis of NaBH₄-treated bio-BG (proposed compounds).

| | | | [1,3-BG | [1,3-BG | | | |
|---|---|---|---|---|---|---|---|
| | | [4-OH-2- | (BDO) | (BDO) | | | [1-Hydroxy- |
| | [3-OH-Butanal] | butanone] | Heavies] | Lights] | [Purity] | [12PDO] | 2-Propanone] |
| Sample | ppm | ppm | % | % | % | ppm | ppm |
| Feed | 237.99 | 239.395 | 0.645 | 0.025 | 99.35 | 17.225 | 7.215 |
| Product | 23.25 | 21.78 | 1.09 | 0.04 | 98.9 | 31.55 | 0 |

SPME analysis demonstrated that the levels of tested ketones and aldehydes (proposed compounds) in bio-BG samples were substantially reduced by NaBH₄-treatments. GEMS purity analysis also confirmed that the bio-BG concentrations of 3-hydroxy-butanal and 4-hydroxy-2-butanone (proposed compounds) were reduced 10-fold, while yielding the corresponding alcohols. Unknown lights in the bio-BG samples increased by 150 ppm and unknown heavies were found to increase by 4500 ppm. The UV absorbance at 270 nm of NaBH₄-treated bio-BG samples was found to be reduced from 0.429 to 0.048. See, e.g., FIG. 13 (bio-BG preparations #7 vs. #8). The substantial reduction in UV absorbance indicated that most of the absorbance in bio-BG likely was due to aldehydes and ketones, which are selectively reduced by NaBH₄, and not due to conjugated double bond systems, which are not reduced by NaBH₄.

Qualitatively, the smell of NaBH₄-treated bio-BG samples was found to be more intense and offensive.

### Example 10

### ASPEN Modeling of Known Odor-Causing Compounds

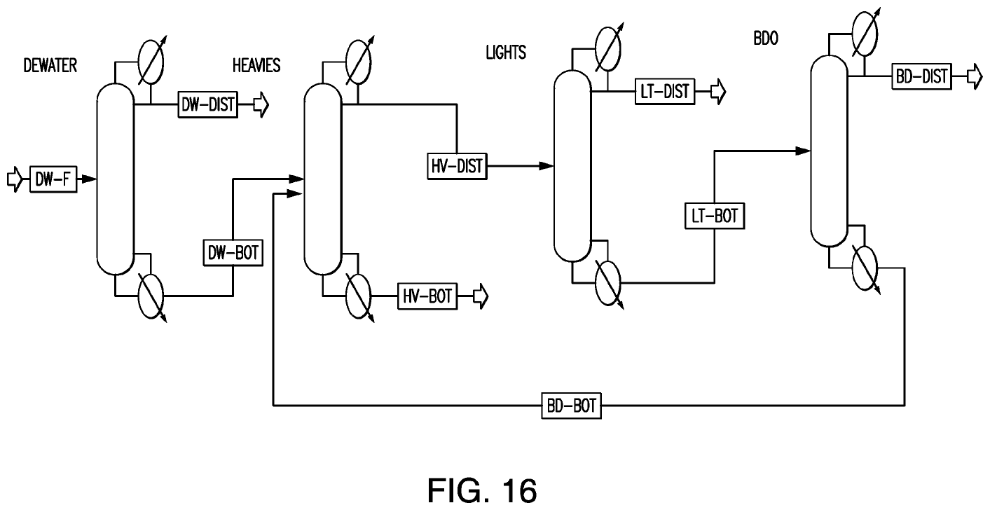

A 4-column distillation simulation was created in ASPEN to understand possible challenges to removing impurities from bioderived 1,3-BG. See also FIG. 16. The following proposed trace contaminants of bioderived 1,3-BG were included in the distillation simulation:
2,3-Butanediol
1,2-Propanediol
Acetaldol (3-hydroxy-butanal)
4-OH-2-Butanone

In the model, the vacuum for the dewatering column was set to 80 torr and the bottoms temperature was estimated to be 144° C. The vacuum of the three following distillation columns was set to 25 torr in each column and the bottoms temperatures were estimated at 118-119° C.

The ASPEN modeling results showed that all of the water, 3-hydroxy-butanal and 4-hydroxy-2-butanone and a small amount of 2,3-BDO were removed from the bioderived 1,3-BG containing product stream as dewater distillate. The balance of lights impurities (remaining 2,3-BDO and 1,2-PDO) were found to be removed in the lights column. These findings are consistent with the boiling point differences of the modeled trace contaminants, e.g., as listed in Table 7. No azeotropes were observed.

### Other Alternative Embodiments

Although the invention has has been described with reference to the embodiments and examples provided above, it should be understood that various modifications can be made without departing from the spirit of the invention.

1. Bioderived 1,3-butylene glycol (1,3-BG), wherein the bioderived 1,3-BG comprises detectable levels of one or more compounds selected from the group consisting of 3-hydroxy-butanal, 4-hydroxy-2-butanone, 4-(3-hydroxybutoxy)butan-2-one, 4-((4-hydroxybutan-2-yl)oxy)-butan-2-one, 1,2-propanediol, 1,3-propanediol and 2,3-butanediol.

2. (canceled)

3. The bioderived 1,3-BG of claim 1, wherein the bioderived 1,3-BG comprises higher levels of one or more compound selected from the group consisting of 3-hydroxy-butanal, 4-hydroxy-2-butanone, 4-(3-hydroxybutoxy)butan-2-one and 4-((4-hydroxybutan-2-yl)oxy)-butan-2-one compared to petro-BG.

4. The bioderived 1,3-BG of claim 1, wherein the chiral purity of the bioderived 1,3-BG is 95% or more, 96% or more, 97% or more, 98% or more, 99.0% or more, 99.1% or more, 99.2% or more, 99.3% or more, 99.4% or more, 99.5% or more, 99.6% or more, 99.7% or more, 99.8% or more, or 99.9% or more, and wherein the bioderived 1,3-BG has a chemical purity of 99.0% or more, 99.1% or more, 99.2% or more, 99.3% or more, 99.4% or more, 99.5% or more, 99.6% or more, 99.7% or more, 99.8% or more, or 99.9% or more.

5. (canceled)

6. The bioderived 1,3-BG of claim 1, wherein the bioderived 1,3-BG comprises more R-enantiomer than S-enantiomer.

7. The bioderived 1,3-BG of claim 6, wherein the bioderived 1,3-BG has a chiral purity of 95% or more and a chemical purity of 99.0% or more, a chiral purity of 99.0% or more and a chemical purity of 99.0% or more, or a chiral purity of 99.5% or more and a chemical purity of 99.0% or more.

8. (canceled)

9. (canceled)

10. The bioderived 1,3-BG of claim 1, wherein the bioderived 1,3-BG is industrial grade or cosmetic grade.

11. The bioderived 1,3-BG of claim 1, wherein the bioderived 1,3-BG comprises levels of 5 ppm or more, 10 ppm or more, 20 ppm or more, 30 ppm or more, 40 ppm or more or more, 50 ppm or more, 100 ppm or more, 200 ppm or more, 300 ppm or more, 400 ppm or more, 500 ppm or more, 600 ppm or more, 700 ppm or more, 800 ppm or more, 900 ppm or more, 1,000 ppm or more, 1,500 ppm or more, or 2,000 ppm or more of the compound.

12. The bioderived 1,3-BG of claim 1, wherein the bioderived 1,3-BG comprises detectable levels of a compound characterized by a mass spectrum according to FIG. 3 or FIG. 4.

13. The bioderived 1,3-BG of claim 1, wherein the bioderived 1,3-BG comprises a compound detectable in a GC-MS chromatogram as a peak eluting with a relative retention time of between 0.97-0.99, wherein the relative retention time of 1,3-BG is 1.0.

14. The bioderived 1,3-BG of claim 1, wherein the bioderived 1,3-BG comprises a compound detectable in a GC-MS chromatogram as a peak eluting with a relative retention time of between 0.94-0.96, wherein the relative retention time of 1,3-BG is 1.0.

15. The bioderived 1,3-BG of claim 1, wherein the bioderived 1,3-BG does not comprise detectable levels of one or more contaminants of petro-BG detectable in an GC-MS chromatogram as peaks eluting with a relative retention time of between 0.8-0.95, wherein the relative retention time of 1,3-BG is 1.0.

16. (canceled)

17. The bioderived 1,3-BG of claim 1, wherein the overall purity of the bioderived 1,3-BG is 99% or higher, the overall level of heavies is 0.8% or less, and the overall level of lights is 0.2% or less.

18. The bioderived 1,3-BG of claim 1, wherein the UV absorbance between 220 nm and 260 nm of the bioderived 1,3-BG is at least 2-fold, at least 3-fold, at least 4-fold, at least 5-fold, at least 6-fold, at least 7-fold, at least 8-fold, at least 9-fold, or at least 10-fold lower than the UV absorbance of petro-BG, and wherein the bioderived 1,3-BG does not comprise detectable levels of 1-4-(4-methyl-1,3-dioxan-2-yl)propan-2-one.

19. (canceled)

20. The bioderived 1,3-BG of claim 1, wherein the bioderived 1,3-BG comprises at least 2-fold, at least 3-fold, at least 4-fold, at least 5-fold, at least 6-fold, at least 7-fold, at least 8-fold, at least 9-fold, or at least 10-fold lower levels of 1-4-(4-methyl-1,3-dioxan-2-yl)propan-2-one than petro-BG.

21. (canceled)

22. (canceled)

23. A process of purifying bioderived 1,3-BG comprising:
(a) subjecting a first bioderived 1,3-BG-containing product stream to a first column distillation procedure to remove materials with a boiling point higher than bioderived 1,3-BG, as a first high boilers stream, to produce a second bioderived 1,3-BG-containing product stream;
(b) subjecting the second bioderived 1,3-BG-containing product stream to a second column distillation procedure to remove materials with a boiling point lower than bioderived 1,3-BG, to produce a third bioderived 1,3-BG-containing product stream; and
(c) subjecting the third bioderived 1,3-BG-containing product stream to a third column distillation procedure to remove materials with boiling points higher than bioderived 1,3-BG as a second high-boilers stream, to produce a purified bioderived 1,3-BG product.

24. The process of claim 23, further comprising subjecting a crude bioderived 1,3-BG mixture to a dewatering column distillation procedure to remove materials with a boiling point lower than bioderived 1,3-BG from the crude bioderived 1,3-BG mixture to produce the first bioderived 1,3-BG-containing product stream of (a).

25. The process of claim 23, further comprising subjecting crude bioderived 1,3-BG to polishing ion exchange to produce the first bioderived 1,3-BG-containing product stream of (a).

26. The process of claim 25, wherein the purified bioderived 1,3-BG product comprises detectable levels of one or more compounds selected from the group consisting of 3-hydroxy-butanal, 4-hydroxy-2-butanone, 4-(3-hydroxybutoxy)butan-2-one, 4-((4-hydroxybutan-2-yl)oxy)-butan-2-one, 1,2-propanediol, 1,3-propanediol and 2,3-butanediol.

27. The process of claim 25, wherein the purified bioderived 1,3-BG product does not comprise a detectable level, or only comprises a low level, of 1-4-(4-methyl-1,3-dioxan-2-yl)propan-2-one.

28. The process of claim 25, further comprising adding a base to a bioderived 1,3-BG-containing product stream before or after any one of (a), (b), or (c).

29. (canceled)

30. The process of claim 25, further comprising treating a bioderived 1,3-BG containing product stream with a hydrogenation reaction before or after any one of (a), (b), or (c).

31. (canceled)

32. The process of claim 30, wherein the hydrogenation reaction reduces the concentration of 3-hydroxy-butanal or 4-hydroxy-2-butanone in the second bioderived 1,3-BG containing product stream by 50% or more, 60% or more, 70% or more, 80% or more, 90% or more, or 95% or more, and wherein the hydrogenation reaction reduces the UV absorption at 270 nm or at 220 nm in the second bioderived 1,3-BG containing product stream by 50% or more, 60% or more, 70% or more, 80% or more, 90% or more, or 95% or more.

33. (canceled)

34. (canceled)

35. The process of claim 25, wherein the purified bioderived 1,3-BG product is collected as a distillate of the third column distillation procedure and wherein (c) further comprises contacting the distillate of the third column distillation procedure with activated carbon to produce the purified bioderived 1,3-BG product.

36. (canceled)

37. The process of claim 25, further comprising contacting the second bioderived 1,3-BG containing product stream with activated carbon prior to performing step (c), wherein the contacting with activated carbon reduces the concentration of 3-hydroxy-butanal or 4-hydroxy-2-butanone in the second bioderived 1,3-BG containing product stream by 50% or more, 60% or more, 70% or more, 80% or more, 90% or more, or 95% or more.

38. (canceled)

39. The process of claim 25, further comprising contacting the second bioderived 1,3-BG containing product stream with sodium borohydride (NaBH₄) prior to performing step (c), wherein the contacting with NaBH₄ reduces the UV absorption at 270 nm or at 220 nm in the second bioderived 1,3-BG containing product stream by 50% or more, 60% or more, 70% or more, 80% or more, 90% or more, or 95% or more.

40. The process of claim 23, wherein bioderived 1,3-BG has a chiral purity of 55% or more and wherein the purified bioderived 1,3-BG product has a chemical purity of 99.0% or more.

41. (canceled)

42. A system for purifying bioderived 1,3-BG, comprising:
a first distillation column receiving a first bioderived 1,3-BG containing product stream generating a first stream of materials with boiling points higher than 1,3-BG, and a second bioderived 1,3-BG-containing product stream;
a second distillation column receiving the second bioderived 1,3-BG-containing product stream generating a stream of materials with boiling points lower than 1,3-BG, and a third bioderived 1,3-BG-containing product stream; and
a third distillation column receiving the third 1,3-BG-containing product stream at a feed point and generating a second stream of materials with boiling points higher than 1,3-BG, and a fourth bioderived 1,3-BG-containing product stream comprising a purified bioderived 1,3-BG product.

43. (canceled)

44. The system of claim 42, comprising a polishing column receiving a crude bioderived 1,3-BG mixture generating a crude bioderived 1,3-BG mixture of reduced salt content, wherein the polishing column is an ion exchange chromatography column.

45. (canceled)

46. The system of claim 42, comprising a dewatering column receiving a crude bioderived 1,3-BG mixture generating a stream of materials with boiling points lower than 1,3-BG and the first bioderived 1,3-BG-containing product stream.

47. (canceled)
